(19) United States
(12) Patent Application Publication
(10) Pub. No.: US 2006/0063752 A1
(43) Pub. Date: Mar. 23, 2006

(54) Bicyclic heterocycles, pharmaceutical compositions containing them, their use, and processes for preparing them

(75) Inventors: Frank Himmelsbach, Mittelbiberach (DE); Elke Langkopf, Warthausen (DE); Stefan Blech, Warthausen (DE); Birgit Jung, Schwabenheim (DE); Thomas Metz, Vienna (AT); Flavio Solca, (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/266,920

(22) Filed: Nov. 4, 2005

Related U.S. Application Data
(63) Continuation of application No. 09/938,235, filed on Aug. 23, 2001.
Application No. 09/938,235 is a continuation of application No. PCT/EP00/02228, filed on Mar. 14, 2000.

(51) Int. Cl. A61K 31/554; A61K 31/517; A61K 31/47

(52) U.S. Cl. 514/211.05; 514/266.4; 514/313; 544/292; 546/159

(57) ABSTRACT

A compound of formula (I) 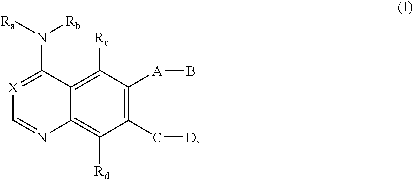
wherein Rₐ, R_{b}, R_{c}, A, B, C, D, and X are as defined herein, or a tautomer, stereoisomer, or salt thereof, particularly a physiologically acceptable salt thereof. In addition, pharmaceutical compositions comprising an effective amount of a compound of formula (I), methods for the treatment or prophylaxis of benign or malignant tumors, diseases of the airways and lungs, polyps, diseases of the gastrointestinal tract, bile duct, gall bladder, kidneys, and skin, and methods for making compounds of formula (I) are disclosed.

### RELATED APPLICATIONS

This application is a continuation of International Application No. PCT/EP00/02228, filed on 14 Mar. 2000, benefit of which is hereby claimed, pursuant to 35 U.S.C. § 365(c) and § 120.

### SUMMARY OF THE INVENTION

The present invention relates to bicyclic heterocyclic compounds of general formula (I)

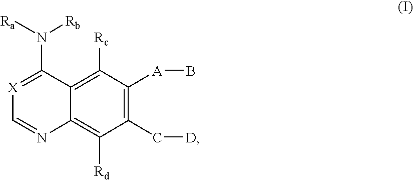

the tautomers, the stereoisomers, and the salts thereof, particularly the physiologically acceptable salts thereof with inorganic or organic acids or bases which have valuable pharmacological properties, particularly an inhibiting effect on the signal transduction mediated by tyrosine kinases, their use in treating diseases, particularly tumoral diseases, diseases of the lungs and respiratory tract and the preparation thereof.

In the above general formula (I)

Rₐ denotes a hydrogen atom or a C₁₋₄-alkyl group,

R_{b} denotes a phenyl, benzyl, or 1-phenylethyl group wherein the phenyl nucleus is substituted in each case by the groups R₁ to R₃, wherein:
R₁ and R₂, which may be identical or different, each denote a hydrogen, fluorine, chlorine, bromine, or iodine atom,
a C₁₋₄-alkyl, hydroxy, C₁₋₄-alkoxy, C₃₋₆-cycloalkyl, C₄₋₆-cycloalkoxy, C₂₋₅-alkenyl, or C₂₋₅-alkynyl group,
an aryl, aryloxy, arylmethyl, or arylmethoxy group,
a C₃₋₅-alkenyloxy or C₃₋₅-alkynyloxy group, wherein the unsaturated moiety may not be linked to the oxygen atom,
a C₁₋₄-alkylsulfenyl, C₁₋₄-alkylsulfinyl, C₁₋₄-alkylsulfonyl, C₁₋₄-alkylsulfonyloxy, trifluoromethylsulfenyl, trifluoromethylsulfinyl, or trifluoromethylsulfonyl group,
a methyl or methoxy group substituted by 1 to 3 fluorine atoms,
an ethyl or ethoxy group substituted by 1 to 5 fluorine atoms,
a cyano or nitro group or an amino group optionally substituted by one or two C₁₋₄-alkyl groups, wherein the substituents may be identical or different,
or R₁ together with R₂, if they are bound to adjacent carbon atoms, denote a —CH═CH—CH═CH, —CH═CH—NH, or —CH═N—NH group, and
R₃ denotes a hydrogen, fluorine, chlorine, or bromine atom,
a C₁₋₄-alkyl, trifluoromethyl, or C₁₋₄-alkoxy group,

R_{c} and R_{d}, which may be identical or different, each denote a hydrogen, fluorine, or chlorine atom, or a methoxy group, or a methyl group optionally substituted by a methoxy, dimethyl-amino, diethylamino, pyrrolidino, piperidino, or morpholino group,

X denotes a methine group substituted by a cyano group or a nitrogen atom,

A denotes an —O—C₁₋₆-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom of the abovementioned groups in each case is linked to the bicyclic heteroaromatic ring,

an —O—C₁₋₆-alkylene group which is substituted by an R₆O—CO or R₆O—CO—C₁₋₄-alkyl group, wherein R₆ is as hereinafter defined and the oxygen atom of the abovementioned —O—C₁₋₆-alkylene groups in each case is linked to the bicyclic heteroaromatic ring,

an —O—C₂₋₆-alkylene group which is substituted from position 2 onwards by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group and the oxygen atom of the abovementioned-O—C₂₋₆-alkylene groups in each case is linked to the bicyclic heteroaromatic ring,

a —C₁₋₆-alkylene group,

an —NR₄—C₁₋₆-alkylene, —NR₄—C₃₋₇-cycloalkylene, —NR₄—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety of the abovementioned groups in each case is linked to the bicyclic heteroaromatic ring, and
R₄ denotes a hydrogen atom or a C₁₋₄-alkyl group,

an oxygen atom, this being linked to a carbon atom of the group B, or

a NR₄ group, the latter being linked to a carbon atom of the group B and R₄ being as hereinbefore defined,

B denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, wherein:
R₅ denotes a hydrogen atom,
a C₁₋₄-alkyl group which may be substituted by an R₆O—CO, (R₇O—PO—OR₈), or (R₇O—PO—R₉) group,
a C₂₋₄-alkyl group which is substituted from position 2 by a hydroxy, C₁₋₄-alkoxy, amino, C₁₋₄-alkylamino, or di-(C₁₋₄-alkyl)-amino group or by a 4- to 7-membered alkyleneimino group, wherein in the abovementioned 6- to 7-membered alkyleneimino groups in each case a methylene group in the 4 position may be replaced by an oxygen or sulfur atom, or by a sulfinyl, sulfonyl, imino, or N—(C₁₋₄-alkyl)-imino group,
a C₃₋₇-cycloalkyl or C₃₋₇-cycloalkyl-C₁₋₃-alkyl group,
R₆, R₇, and R₈, which may be identical or different, in each case denote a hydrogen atom,
a C₁₋₈-alkyl group which may be substituted from position 2 onwards by a hydroxy, C₁₋₄-alkoxy, amino, C₁₋₄-alkylamino, or di-(C₁₋₄-alkyl)-amino group or by a 4- to 7-membered alkyleneimino group, wherein in the abovementioned 6- to 7-membered alkyleneimino groups in each case a methylene group in the 4 position may be replaced by an oxygen or sulfur atom, or by a sulfinyl, sulfonyl, imino, or N—(C₁₋₄-alkyl)-imino group,
a C₄₋₇-cycloalkyl group optionally substituted by 1 or 2 methyl groups,
a C₃₋₅-alkenyl or C₃₋₅-alkynyl group, wherein the unsaturated moiety may not be linked to the oxygen atom,
a C₃₋₇-cycloalkyl-C₁₋₄-alkyl, aryl, aryl-C₁₋₄-alkyl, or R_{g}CO—O—(RₑCR_{f}) group, wherein:
   Rₑ and R_{f}, which may be identical or different, in each case denote a hydrogen atom or a C₁₋₄-alkyl group, and
   R_{g} denotes a C₁₋₄-alkyl, C₃₋₇-cycloalkyl, C₁₋₄-alkoxy, or C₅₋₇-cycloalkoxy group,
   and R₉ denotes a C₁₋₄-alkyl, aryl, or aryl-C₁₋₄-alkyl group,

a 4- to 7-membered alkyleneimino group which is substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a 4- to 7-membered alkyleneimino group which is substituted by two R₆O—CO or R₆O—CO—C₁₋₄-alkyl groups or by an R₆OCO group and an R₆O—CO—C₁₋₄-alkyl group, wherein R₆ is as hereinbefore defined,
a piperazino or homopiperazino group which is substituted in the 4 position by the group R₁₀ and additionally at a cyclic carbon atom by an R₆O—CO, (R₇O—PO—OR₈), (R₇—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined, and
R₁₀ denotes a hydrogen atom, or a C₁₋₄-alkyl, formyl, C₁₋₄-alkylcarbonyl, or C₁₋₄-alkylsulfonyl group,

a piperazino or homopiperazino group which is substituted in the 4 position by the group R₁₀ and is additionally substituted at cyclic carbon atoms by two R₆O—CO or R₆O—CO—C₁₋₄-alkyl groups or by an R₆O—CO group and an R₆O—CO—C₁₋₄-alkyl group wherein R₆ and R₁₀ are as hereinbefore defined,

a piperazino or homopiperazino group which is substituted in each case in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a piperazino or homopiperazino group which is substituted in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group and is additionally substituted at cyclic carbon atoms by one or two R₆O—CO or R₆O—CO—C₁₋₄-alkyl groups or by an R₆O—CO group and an R₆O—CO—C₁₋₄-alkyl group wherein R₆ to R₉ are as hereinbefore defined,

a morpholino or homomorpholino group which is substituted in each case by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a morpholino or homomorpholino group which is substituted by two R₆O—CO or R₆O—CO—C₁₋₄-alkyl groups or by an R₆O—CO group and an R₆O—CO—C₁₋₄-alkyl group wherein R₆ is as hereinbefore defined,

a pyrrolidinyl, piperidinyl or hexahydroazepinyl group substituted in the 1 position by the group R₁₀, wherein the abovementioned 5 to 7-membered rings are in each case additionally substituted at a carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₁₀ are as hereinbefore defined,

a pyrrolidinyl, piperidinyl, or hexahydroazepinyl group substituted in the 1 position by the group R₁₀, wherein the abovementioned 5 to 7-membered rings in each case are additionally substituted at carbon atoms by two R₆O—CO or R₆O—CO—C₁₋₄-alkyl groups or by an R₆O—CO group and an R₆O—CO—C₁₋₄-alkyl group wherein R₆ and R₁₀ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group, wherein the abovementioned 5- to 7-membered rings in each case are additionally substituted at carbon atoms by one or two R₆O—CO or R₆O—CO—C₁₋₄-alkyl groups or by an R₆O—CO group and an R₆O—CO—C₁₋₄-alkyl group wherein R₆ to R₉ are as hereinbefore defined,

a 2-oxomorpholino group which may be substituted by 1 to 4 C₁₋₂-alkyl groups,

a 2-oxomorpholinyl group which is substituted in the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group, wherein R₆ to R₉ are as hereinbefore defined and the abovementioned 2-oxomorpholinyl groups in each case are linked to a carbon atom of the group A,

an R₁₁NR₅ group, wherein R₅ is as hereinbefore defined, and
R₁₁ denotes a 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group optionally substituted by one or two methyl groups,

or A and B together denotes a hydrogen, fluorine, or chlorine atom,

a C₁₋₆-alkoxy group,

a C₂₋₆-alkoxy group which is substituted from position 2 onwards by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, 4-(C₁₋₄-alkyl)-homopiperazino, or 1-imidazolyl group,

a C₁₋₄-alkoxy group which is substituted by a pyrrolidinyl, piperidinyl, or hexahydroazepinyl group substituted in the 1 position by the group R₁₀, wherein R₁₀ is as hereinbefore defined,

a C₁₋₆-alkoxy group which is substituted by an R₆O—CO, (R₇O—PO—OR₈), or (R₇O—PO—R₉) group, wherein R₆ to R₉ are as hereinbefore defined,

a C₃₋₇-cycloalkoxy or C₃₋₇-cycloalkyl-C₁₋₄-alkoxy group,

an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a 2-oxomorpholino group which may be substituted by 1 or 2 methyl groups,

C denotes an —O—C₁₋₆-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group wherein the oxygen atom of the abovementioned group in each case is linked to the bicyclic heteroaromatic ring,

an —O—C₁₋₆-alkylene group which is substituted by an R₆O—CO or R₆O—CO—C₁₋₄-alkyl group, wherein R₆ is as hereinbefore defined and the oxygen atom of the abovementioned-O—C₁₋₆-alkylene groups in each case is linked to the bicyclic heteroaromatic ring,

an —O—C₂₋₆-alkylene group which is substituted from position 2 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group and the oxygen atom of the abovementioned-O—C₂₋₆-alkylene groups in each case is linked to the bicyclic heteroaromatic ring,

a —C₁₋₆-alkylene group,

an —NR₄—C₁₋₆-alkylene, —NR₄—C₃₋₇-cycloalkylene, —NR₄—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety of the abovementioned groups in each case is linked to the bicyclic heteroaromatic ring and R₄ is as hereinbefore defined,

an oxygen atom, which is linked to a carbon atom of the group D, or

a NR₄ group, where the latter is linked to a carbon atom of the group D and R₄ is as hereinbefore defined,

D denotes an R₆—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 6 carbon atoms, may additionally be substituted by one or two C₁₋₂-alkyl groups or by an R₆—CO or R₆—CO—C₁₋₂-alkyl group, wherein R₅ to R₉ are as hereinbefore defined,

a 4- to 7-membered alkyleneimino group which is substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a 4- to 7-membered alkyleneimino group which is substituted by two R₆O—CO or R₆O—CO—C₁₋₄-alkyl groups or by an R₆OCO group and an R₆O—CO—C₁₋₄-alkyl group wherein R₆ is as hereinbefore defined,

a piperazino or homopiperazino group which is substituted in the 4 position by the group R₁₀ and additionally at a cyclic carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (P₇O—PO—R₉)—C₁₋₄-alkyl group wherein R₆ to R₁₀ are as hereinbefore defined,

a piperazino or homopiperazino group which is substituted in the 4 position by the group R₁₀ and is additionally substituted at cyclic carbon atoms by two R₆O—CO or R₆O—CO—C₁₋₄-alkyl groups or by an R₆O—CO group and an R₆O—CO—C₁₋₄-alkyl group wherein R₆ and R₁₀ are as hereinbefore defined,

a piperazino or homopiperazino group which is substituted in each case in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a piperazino or homopiperazino group which is substituted in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group and is additionally substituted at cyclic carbon atoms by one or two R₆O—CO or R₆O—CO—C₁₋₄-alkyl groups or by an R₆O—CO group and an R₆O—CO—C₁₋₄-alkyl group wherein R₆ to R₉ are as hereinbefore defined,

a morpholino or homomorpholino group which is substituted in each case by an R₆O—CO, (R₇O—PO—OR₈), (R₇O—PO—R₉), R₆O—CO—C₁₋₄-alkyl, bis-(R₆—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a morpholino or homomorpholino group which is substituted by two R₆O—CO or R₆O—CO—C₁₋₄-alkyl groups or by an R₆O—CO group and an R₆O—CO—C₁₋₄-alkyl group wherein R₆ is as hereinbefore defined,

a pyrrolidinyl, piperidinyl, or hexahydroazepinyl group substituted in the 1 position by the group R₁₀, wherein the abovementioned 5- to 7-membered rings in each case are additionally substituted at a carbon atom by an R₆O—CO, (R₇O—P—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₁₀ are as hereinbefore defined,

a pyrrolidinyl, piperidinyl, or hexahydroazepinyl group substituted in the 1 position by the group R₁₀, wherein the abovementioned 5- to 7-membered rings are in each case additionally substituted at carbon atoms by two R₆O—CO or R₆O—CO—C₁₋₄-alkyl groups or by an R₆O—CO group and an R₆O—CO—C₁₋₄-alkyl group wherein R₆ and R₁₀ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group, wherein the abovementioned 5- to 7-membered rings are in each case additionally substituted at carbon atoms by one or two R₆O—CO or R₆O—CO—C₁₋₄-alkyl groups or by an R₆O—CO group and an R₆O—CO—C₁₋₄-alkyl group wherein R₆ to R₉ are as hereinbefore defined,

a 2-oxomorpholino group which may be substituted by 1 to 4 C₁₋₂-alkyl groups,

a 2-oxomorpholinyl group which is substituted in the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined and the abovementioned 2-oxomorpholinyl groups are in each case linked to a carbon atom of the group C,

an R₁₁NR₅ group wherein R₅ and R₁₁ are as hereinbefore defined, or

C and D together denote a hydrogen, fluorine, or chlorine atom,

a C₁₋₆-alkoxy group,

a C₂₋₆-alkoxy group which is substituted from position 2 by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, 4-(C₁₋₄-alkyl)-homopiperazino, or 1-imidazolyl group,

a C₁₋₄-alkoxy group which is substituted by a pyrrolidinyl, piperidinyl, or hexahydroazepinyl group substituted in the 1 position by the group R₁₀, wherein R₁₀ is as hereinbefore defined,

a C₁₋₆-alkoxy group which is substituted by an R₆O—CO, (R₇O—PO—OR₈), or (R₇O—PO—R₉) group, wherein R₆ to R₉ are as hereinbefore defined,

a C₃₋₇-cycloalkoxy or C₃₋₇-cycloalkyl-C₁₋₄-alkoxy group

an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a 2-oxomorpholino group which may be substituted by 1 or 2 methyl groups,

with the proviso that at least one of the groups B or D or A together with B or C together with D contains an optionally substituted 2-oxomorpholinyl group, an (R₇O—PO—OR₈) or (R₇O—PO—R₉) group, or that at least one of the groups B or D contains an optionally substituted 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group, or

that at least one of the groups A, B, C, or D, or A together with B, or C together with D contains an R₆O—CO group and additionally one of the groups A, B, C, or D, or A together with B, or C together with D contains a primary, secondary, or tertiary amino function, wherein the nitrogen atom of this amino function is not linked to a carbon atom of an aromatic group.

By the aryl moieties mentioned in the definition of the abovementioned groups is meant a phenyl group which may in each case be monosubstituted by R₁₂, mono-, di-, or trisubstituted by R₁₃ or monosubstituted by R₁₂ and additionally mono- or disubstituted by R₁₃, wherein the substituents may be identical or different, and
R₁₂ denotes a cyano, carboxy, C₁₋₄-alkoxycarbonyl, aminocarbonyl, C₁₋₄-alkylaminocarbonyl, di-(C₁₋₄-alkyl)-aminocarbonyl, C₁₋₄-alkylsulfenyl, C₁₋₄-alkylsulfinyl, C₁₋₄-alkylsulfonyl, hydroxy, C₁₋₄-alkylsulfonyloxy, trifluoromethyloxy, nitro, amino, C₁₋₄-alkylamino, di-(C₁₋₄-alkyl)-amino, C₁₋₄-alkylcarbonylamino, N—(C₁₋₄-alkyl)-C₁₋₄-alkylcarbonylamino, C₁₋₄-alkylsulfonylamino, N—(C₁₋₄-alkyl)-C₁₋₄-alkylsulfonylamino, aminosulfonyl, C₁₋₄-alkylaminosulfonyl, or di-(C₁₋₄-alkyl)-aminosulfonyl group or a carbonyl group which is substituted by a 5- to 7-membered alkyleneimino group, wherein in the abovementioned 6- to 7-membered alkyleneimino groups in each case a methylene group in the 4 position may be replaced by an oxygen or sulfur atom, or by a sulfinyl, sulfonyl, imino, or N—(C₁₋₄-alkyl)-imino group, and

R₁₃ denotes a fluorine, chlorine, bromine, or iodine atom, or a C₁₋₄-alkyl, trifluoromethyl, or C₁₋₄-alkoxy group or
two groups R₁₃, if they are bound to adjacent carbon atoms, together denote a C₃₋₅-alkylene, methylenedioxy, or 1,3-butadien-1,4-ylene group,

wherein of the abovementioned compounds the preferred ones are those wherein:

Rₐ to R_{d}, A, and X are as hereinbefore defined,

B denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group,

a 4- to 7-membered alkyleneimino group which is substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a piperazino or homopiperazino group which is substituted in the 4 position by the group R₁₀ and additionally at a cyclic carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a piperazino or homopiperazino group which in each case is substituted in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a pyrrolidinyl, piperidinyl, or hexahydroazepinyl group substituted in the 1 position by the group R₁₀, wherein the abovementioned 5- to 7-membered rings in each case are additionally substituted at a carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a 2-oxomorpholino group which may be substituted by 1 or 2 methyl groups,

a 2-oxomorpholinyl group which is substituted in the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group, wherein R₆ to R₉ are as hereinbefore defined and the abovementioned 2-oxomorpholinyl groups in each case are linked to a carbon atom of the group A, or

A and B together denote a hydrogen, fluorine, or chlorine atom,

a C₁₋₆-alkoxy group,

a C₂₋₆-alkoxy group which is substituted from position 2 by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a C₁₋₆-alkoxy group which is substituted by an R₆O—CO, (R₇O—PO—OR₈), or (R₇O—PO—R₉) group,

a C₄₋₇-cycloalkoxy or C₃₋₇-cycloalkyl-C₁₋₄-alkoxy group,

an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a 2-oxomorpholino group which may be substituted by 1 or 2 methyl groups,

C denotes an —O—C₁₋₆-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom of the abovementioned group in each case is linked to the bicyclic heteroaromatic ring,

an —O—C₁₋₆-alkylene group which is substituted by an R₆O—CO or R₆O—CO—C₁₋₄-alkyl group, wherein R₆ is as hereinbefore defined,

an —O—C₂₋₆-alkylene group which is substituted from position 2 onwards by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group,

a —C₁₋₆-alkylene group,

an —NR₄—C₁₋₆-alkylene, —NR₄—C₃₋₇-cycloalkylene, —NR₄—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety of the abovementioned groups in each case is linked to the bicyclic heteroaromatic ring,

an oxygen atom, which is linked to a carbon atom of the group D, or

a NR₄ group, this being linked to a carbon atom of the group D, and

D denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group,

a 4- to 7-membered alkyleneimino group which is substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a piperazino or homopiperazino group which is substituted in the 4 position by the group R₁₀ and additionally at a cyclic carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a piperazino or homopiperazino group which is substituted in each case in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄alkyl group,

a pyrrolidinyl, piperidinyl, or hexahydroazepinyl group substituted in the 1 position by the group R₁₀, wherein the abovementioned 5- to 7-membered rings in each case are additionally substituted at a carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)_{C}₁₋₄-alkyl, or (R₇O—PO—R₉)—C₁₋₄-alkyl group,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,

a 2-oxomorpholino group which may be substituted by 1 or 2 methyl groups,

a 2-oxomorpholinyl group which is substituted in the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined and the abovementioned 2-oxomorpholinyl groups are in each case linked to a carbon atom of the group C, or

C and D together denote a hydrogen, fluorine, or chlorine atom,

a C₁₋₆-alkoxy group,

a C₂₋₆-alkoxy group which is substituted from position 2 by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a C₁₋₆-alkoxy group which is substituted by an R₆O—CO, (R₇O—PO—OR₈), or (R₇O—PO—R₉) group,

a C₄₋₇-cycloalkoxy or C₃₋₇-cycloalkyl-C₁₋₄-alkoxy group,

an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group,

a 2-oxomorpholino group which may be substituted by 1 or 2 methyl groups,

with the proviso that at least one of the groups B or D, or A together with B, or C together with D contains an optionally substituted 2-oxomorpholinyl group, a (R₇O—PO—OR₈) or (R₇O—PO—R₉) group, or

that at least one of the groups A, B, C, or D, or A together with B, or C together with D contains an R₆O—CO group and additionally one of the groups A, B, C, or D, or A together with B, or C together with D contains a primary, secondary, or tertiary amino function, wherein the nitrogen atom of this amino function is not linked to a carbon atom of an aromatic group,

wherein in the abovementioned groups A to D R₄ to R₁₀ are as hereinbefore defined,

particularly those compounds wherein:

Rₐ denotes a hydrogen atom,

R_{b} denotes a phenyl, benzyl, or 1-phenylethyl group wherein the phenyl nucleus is substituted in each case by the groups R₁ to R₃, wherein:
R₁ and R₂, which may be identical or different, each denote a hydrogen, fluorine, chlorine, bromine, or iodine atom,
a methyl, ethyl, hydroxy, methoxy, ethoxy, amino, cyano, vinyl, or ethynyl group,
an aryl, aryloxy, arylmethyl, or arylmethoxy group,
a methyl or methoxy group substituted by 1 to 3 fluorine atoms or
R₁ together with R₂, if they are bound to adjacent carbon atoms, denote a —CH═CH—CH═CH, —CH═CH—NH, or —CH═N—NH group, and
R₃ denotes a hydrogen, fluorine, chlorine, or bromine atom,

R_{c} and R_{d} in each case denote a hydrogen atom,

X denotes a nitrogen atom,

A denotes an —O—C₁₋₄-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom of the abovementioned groups in each case is linked to the bicyclic heteroaromatic ring,

an —O—C₂₋₄-alkylene group which is substituted from position 2 onwards by a hydroxy group, wherein the oxygen atom of the abovementioned-O—C₂₋₄-alkylene groups in each case is linked to the bicyclic heteroaromatic ring, or

an oxygen atom, this being linked to a carbon atom of the group B,

B denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, may additionally be substituted by one or two C₁₋₂-alkyl groups or by an R₆O—CO or R₆O—CO—C₁₋₂-alkyl group, wherein:
R₅ denotes a hydrogen atom,
a C₁₋₄-alkyl group which may be substituted by an R₆O—CO group,
a C₂₋₄-alkyl group which is substituted from position 2 by a hydroxy or C₁₋₄-alkoxy group,
a C₃₋₆-cycloalkyl or C₃₋₆-cycloalkyl-C₁₋₃-alkyl group,
R₆, R₇, and R₈, which may be identical or different, in each case denote a hydrogen atom,
a C₁₋₈-alkyl group which may be substituted from position 2 onwards by a hydroxy, C₁₋₄-alkoxy, or di-(C₁₋₄-alkyl)-amino group or by a 4- to 7-membered alkyleneimino group, wherein in the abovementioned 6- to 7-membered alkyleneimino groups in each case a methylene group in the 4 position may be replaced by an oxygen atom or by an N—(C₁₋₂-alkyl)-imino group,
a C₄₋₆-cycloalkyl group,
a C₃₋₅-alkenyl or C₃₋₅-alkynyl group, wherein the unsaturated moiety may not be linked to the oxygen atom,
a C₃₋₆-cycloalkyl-C₁₋₄-alkyl, aryl, aryl-C₁₋₄-alkyl, or R_{g}CO—O—(RₑCR_{f}) group, wherein:
   Rₑ and R_{f}, which may be identical or different, in each case denote a hydrogen atom or a C₁₋₄-alkyl group, and
   R_{g} denotes a C₁₋₄-alkyl, C₃₋₆-cycloalkyl, C₁₋₄-alkoxy, or C₅₋₆-cycloalkoxy group,
and R₉ denotes a C₁₋₄-alkyl group,

a 4- to 7-membered alkyleneimino group which is substituted by an R₆O—CO, R₆O—CO—C₁₋₄-alkyl, or bis-(R₆O—CO)—C₁₋₄-alkyl group wherein R₆ is as hereinbefore defined,
a 4- to 7-membered alkyleneimino group which is substituted by two R₆O—CO or R₆O—CO—C₁₋₄-alkyl groups wherein R₆ is as hereinbefore defined,
a piperazino or homopiperazino group which is substituted in the 4 position by the group R₁₀ and additionally at a cyclic carbon atom by an R₆O—CO, R₆O—CO—C₁₋₄-alkyl, or bis-(R₆O—CO)—C₁₋₄-alkyl group wherein R₆ is as hereinbefore defined, and
R₁₀ denotes a hydrogen atom, or a methyl or ethyl group,

a piperazino or homopiperazino group which is substituted in the 4 position by the group R₁₀ and is additionally substituted at cyclic carbon atoms by two R₆O—CO or R₆O—CO—C₁₋₄-alkyl groups wherein R₆ and R₁₀ are as hereinbefore defined,

a piperazino or homopiperazino group which in each case is substituted in the 4 position by an R₆O—CO—C₁₋₄-alkyl, bis-(R₆O—CO)—C₁₋₄-alkyl, (R₇O—PO_{OR}₈)—C₁₋₄-alkyl, or (R₇O—PO—R₉)—C₁₋₄-alkyl group wherein R₆ to R₉ are as hereinbefore defined,

a piperazino or homopiperazino group which is substituted in the 4 position by an R₆O—CO—C₁₋₄-alkyl or bis-(R₆O—CO)—C₁₋₄-alkyl group and is additionally substituted at cyclic carbon atoms by one or two R₆O—CO or R₆O—CO—C₁₋₄-alkyl groups wherein R₆ is as hereinbefore defined,

a morpholino or homomorpholino group which is substituted in each case by an R₆O—CO, R₆O—CO—C₁₋₄-alkyl, or bis-(R₆O—CO)—C₁₋₄-alkyl group wherein R₆ is as hereinbefore defined,

a morpholino or homomorpholino group which is substituted by two R₆O—CO or R₆O—CO—C₁₋₄-alkyl groups wherein R₆ is as hereinbefore defined,

a pyrrolidinyl, piperidinyl, or hexahydroazepinyl group substituted in the 1 position by the group R₁₀, wherein the abovementioned 5- to 7-membered rings in each case are additionally substituted at a carbon atom by an R₆O—CO, R₆O—CO—C₁₋₄-alkyl, or bis-(R₆O—CO)—C₁₋₄-alkyl group wherein R₆ and R₁₀ are as hereinbefore defined,

a pyrrolidinyl, piperidinyl, or hexahydroazepinyl group substituted in the 1 position by the group R₁₀, wherein the abovementioned 5- to 7-membered rings are in each case additionally substituted at carbon atoms by two R₆O—CO or R₆O—CO—C₁₋₄-alkyl groups wherein R₆ and R₁₀ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a pyrrolidinyl, piperidinyl, or hexahydroazepinyl group substituted in the 1 position by an R₆O—CO—C₁₋₄-alkyl or bis-(R₆O—CO)—C₁₋₄-alkyl group, wherein the abovementioned 5- to 7-membered rings are in each case additionally substituted at carbon atoms by one or two R₆O—CO or R₆O—CO—C₁₋₄-alkyl groups wherein R₆ is as hereinbefore defined,

a 2-oxomorpholino group which may be substituted by 1 to 4 C₁₋₂-alkyl groups,

a 2-oxomorpholinyl group which is substituted in the 4 position by a hydrogen atom, or by a C₁₋₄-alkyl or R₆O—CO—C₁₋₄-alkyl group, wherein R₆ is as hereinbefore defined and the above-mentioned 2-oxomorpholinyl groups in each case are linked to a carbon atom of the group A,

an R₁₁NR₅ group wherein R₅ is as hereinbefore defined, and
R₁₁ denotes a 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group optionally substituted by one or two methyl groups,

or A and B together denote a hydrogen atom,

a C₁₋₄-alkoxy group,

a C₂₋₄-alkoxy group which is substituted from position 2 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group,

a C₁₋₄-alkoxy group which is substituted by a pyrrolidinyl or piperidinyl group substituted in the 1 position by the group R₁₀, wherein R₁₀ is as hereinbefore defined,

a C₁₋₄-alkoxy group which is substituted by an R₆O—CO group, wherein R₆ is as hereinbefore defined,

a C₄₋₇-cycloalkoxy or C₃₋₇-cycloalkyl-C₁₋₄-alkoxy group,

C denotes an —O—C₁₋₄-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom of the abovementioned group in each case is linked to the bicyclic heteroaromatic ring,

an —O—C₂₋₄-alkylene group which is substituted from position 2 onwards by a hydroxy group, wherein the oxygen atom of the abovementioned-O—C₂₋₄-alkylene groups in each case is linked to the bicyclic heteroaromatic ring, or

an oxygen atom, which is linked to a carbon atom of the group D,

D denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, may additionally be substituted by one or two C₁₋₂-alkyl groups or by an R₆O—CO or R₆O—CO—C₁₋₂-alkyl group, wherein R₅ to R₉ are as hereinbefore defined,

a 4- to 7-membered alkyleneimino group which is substituted by an R₆O—CO, R₆O—CO—C₁₋₄-alkyl, or bis-(R₆O—CO)—C₁₋₄-alkyl group wherein R₆ is as hereinbefore defined,

a 4- to 7-membered alkyleneimino group which is substituted by two R₆O—CO or R₆O—CO—C₁₋₄-alkyl groups wherein R₆ is as hereinbefore defined,

a piperazino or homopiperazino group which is substituted in the 4 position by the group R₁₀ and additionally at a cyclic carbon atom by an R₆O—CO, R₆O—CO—C₁₋₄-alkyl, or bis-(R₆O—CO)—C₁₋₄-alkyl group wherein R₆ and R₁₀ are as hereinbefore defined,

a piperazino or homopiperazino group which is substituted in the 4 position by the group R₁₀ and is additionally substituted at cyclic carbon atoms by two R₆O—CO or R₆O—CO—C₁₋₄-alkyl groups wherein R₆ and R₁₀ are as hereinbefore defined,

a piperazino or homopiperazino group which is substituted in each case in the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a piperazino or homopiperazino group which is substituted in the 4 position by an R₆O—CO—C₁₋₄-alkyl or bis-(R₆O—CO)—C₁₋₄-alkyl group and is additionally substituted at cyclic carbon atoms by one or two R₆O—CO or R₆O—CO—C₁₋₄-alkyl groups wherein R₆ is as hereinbefore defined,

a morpholino or homomorpholino group which is substituted in each case by an R₆O—CO, R₆O—CO—C₁₋₄-alkyl, or bis-(R₆O—CO)—C₁₋₄-alkyl group wherein R₆ is as hereinbefore defined,

a morpholino or homomorpholino group which is substituted by two R₆O—CO or R₆O—CO—C₁₋₄-alkyl groups wherein R₆ is as hereinbefore defined,

a pyrrolidinyl, piperidinyl, or hexahydroazepinyl group substituted in the 1 position by the group R₁₀, wherein the abovementioned 5- to 7-membered rings in each case are additionally substituted at a carbon atom by an R₆O—CO, R₆O—CO—C₁₋₄-alkyl, or bis-(R₆O—CO)—C₁₋₄-alkyl group wherein R₆ and R₁₀ are as hereinbefore defined,

a pyrrolidinyl, piperidinyl, or hexahydroazepinyl group substituted in the 1 position by the group R₁₀, wherein the abovementioned 5- to 7-membered rings are in each case additionally substituted at carbon atoms by two R₆O—CO or R₆O—CO—C₁₋₄-alkyl groups wherein R₆ and R₁₀ are as hereinbefore defined,

a pyrrolidinyl, piperidinyl, or hexahydroazepinyl group substituted in the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a pyrrolidinyl, piperidinyl, or hexahydroazepinyl group substituted in the 1 position by an R₆O—CO—C₁₋₄-alkyl or bis-(R₆O—CO)—C₁₋₄-alkyl group, wherein the abovementioned 5- to 7-membered rings are in each case additionally substituted at carbon atoms by one or two R₆O—CO or R₆O—CO—C₁₋₄-alkyl groups wherein R₆ is as hereinbefore defined,

a 2-oxomorpholino group which may be substituted by 1 to 4 C₁₋₂-alkyl groups,

a 2-oxomorpholinyl group which is substituted in the 4 position by a hydrogen atom, or by a C₁₋₄-alkyl or R₆O—CO—C₁₋₄-alkyl group, wherein R₆ is as hereinbefore defined and the above-mentioned 2-oxomorpholinyl groups are in each case linked to a carbon atom of the group C,

an R₁₁NR₅ group wherein R₅ and R₁₁ are as hereinbefore defined, or

C and D together denote a hydrogen atom,

a C₁₋₄-alkoxy group,

a C₂₋₄-alkoxy group which is substituted from position 2 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group,

a C₁₋₄-alkoxy group which is substituted by a pyrrolidinyl or piperidinyl group substituted in the 1 position by the group R₁₀, wherein R₁₀ is as hereinbefore defined,

a C₁₋₄-alkoxy group which is substituted by an R₆O—CO group, wherein R₆ is as hereinbefore defined,

a C₄₋₇-cycloalkoxy or C₃₋₇-cycloalkyl-C₁₋₄-alkoxy group

with the proviso that at least one of the groups B or D, or A together with B, or C together with D contains an optionally substituted 2-oxomorpholinyl group, a (R₇O—PO—OR₈) or (R₇O—PO—R₉) group, or

that at least one of the groups B or D contains an optionally substituted 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group, or

that at least one of the groups A, B, C, or D, or A together with B, or C together with D contains an R₆O—CO group and additionally one of the groups A, B, C, or D, or A together with B, or C together with D contains a primary, secondary, or tertiary amino function, wherein the nitrogen atom of this amino function is not linked to a carbon atom of an aromatic group,

wherein by the aryl moieties mentioned in the definition of the abovementioned groups is meant a phenyl group which in each case may be monosubstituted by R₁₂, mono- or di-substituted by R₁₃, or monosubstituted by R₁₂ and additionally mono- or disubstituted by R₁₃, wherein the substituents may be identical or different, and
R₁₂ denotes a cyano, C₁₋₂-alkoxycarbonyl, aminocarbonyl, C₁₋₂-alkylaminocarbonyl, di-(C₁₋₂-alkyl)-aminocarbonyl, C₁₋₂-alkylsulfenyl, C₁₋₂-alkylsulfinyl, C₁₋₂-alkylsulfonyl, hydroxy, nitro, amino, C₁₋₄-alkylamino, or di-(C₁₋₄-alkyl)-amino group, and
R₁₃ denotes a fluorine, chlorine, bromine, or iodine atom, or a C₁₋₂-alkyl, trifluoromethyl, or C₁₋₂-alkoxy group or
two groups R₁₃, if they are bound to adjacent carbon atoms, together denote a C₃₋₅-alkylene, methylenedioxy, or 1,3-butadien-1,4-ylene group,

the tautomers, stereoisomers, and salts thereof.

Particularly preferred compounds of general formula I are those wherein:

Rₐ denotes a hydrogen atom,

R_{b} denotes a phenyl, benzyl, or 1-phenylethyl group wherein the phenyl nucleus is substituted in each case by the groups R₁ to R₃, wherein:
R₁ and R₂, which may be identical or different, each denote a hydrogen, fluorine, chlorine, or bromine atom,
a methyl, trifluoromethyl, methoxy, ethynyl, or cyano group, and
R₃ denotes a hydrogen atom,

R_{c} and R_{d} in each case denote a hydrogen atom,

X denotes a nitrogen atom,

A denotes an —O—C₁₋₄-alkylene or —O—CH₂—CH(OH)—CH₂ group, wherein the oxygen atom of the abovementioned groups in each case is linked to the bicyclic heteroaromatic ring,

B denotes an R₆O—CO-alkylene-NR₅ group wherein the alkylene moiety, which is straight-chained and contains 1 or 2 carbon atoms, may additionally be substituted by an R₆O—CO or R₆O—CO-methyl group, wherein:
R₅ denotes a hydrogen atom,
a C₁₋₂-alkyl group which may be substituted by an R₆O—CO group,
a C₂₋₄-alkyl group which is substituted from position 2 onwards by a hydroxy group,
a C₃₋₆-cycloalkyl or C₃₋₆-cycloalkylmethyl group, and
R₆ denotes a hydrogen atom,
a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group, wherein:
   Rₑ denotes a hydrogen atom or a C₁₋₄-alkyl group,
   R_{f} denotes a hydrogen atom, and
   R_{g} denotes a C₁₋₄-alkyl, cyclopentyl, cyclohexyl, C₁₋₄-alkoxy, cyclopentyloxy, or cyclohexyloxy group,
a pyrrolidino or piperidino group which is substituted by an R₆O—CO or R₆O—CO—C₁₋₂-alkyl group wherein R₆ is as hereinbefore defined,
a pyrrolidino or piperidino group which is substituted by two R₆O—CO or R₆O—CO—C₁₋₂-alkyl groups wherein R₆ is as hereinbefore defined,

a piperazino group which is substituted in the 4 position by the group R₁₀ and additionally at a cyclic carbon atom by an R₆O—CO or R₆O—CO—C₁₋₂-alkyl group wherein R₆ is as hereinbefore defined, and
R₁₀ denotes a hydrogen atom, or a methyl or ethyl group,

a piperazino group which is substituted in the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ is as hereinbefore defined,
R₇ and R₈, which may be identical or different, in each case denote a hydrogen atom, or a methyl, ethyl, phenyl, benzyl, 5-indanyl, or R_{g}CO—O—(RₑCR_{f}) group, wherein:
   Rₑ to R_{g} are as hereinbefore defined,
and R₉ denotes a methyl or ethyl group,

a piperazino group which is substituted in the 4 position by an R₆O—CO—C₁₋₂-alkyl group and additionally at a cyclic carbon atom by an R₆O—CO or R₆O—CO—C₁₋₂-alkyl group wherein R₆ is as hereinbefore defined,

a morpholino group which is substituted by an R₆O—CO or R₆O—CO—C₁₋₂-alkyl group, wherein R₆ is as hereinbefore defined,

a pyrrolidinyl or piperidinyl group substituted in the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ to R₉ are as hereinbefore defined,

a 2-oxomorpholino group which may be substituted by 1 or 2 methyl groups,

a 2-oxomorpholinyl group which is substituted in the 4 position by a methyl, ethyl, or R₆O—CO—C₁₋₂-alkyl group, wherein R₆ is as hereinbefore defined and the abovementioned 2-oxomorpholinyl groups in each case are linked to a carbon atom of the group A, or
a R₁₁N(C₁₋₂-alkyl) group wherein R₁₁ denotes a 2-oxotetrahydrofuran-3-yl or 2-oxotetrahydrofuran-4-yl group, or

A and B together denote a hydrogen atom, or a methoxy, ethoxy, or 2-methoxyethoxy group,

a C₁₋₂-alkoxy group which is substituted by an R₆O—CO group, wherein R₆ is as hereinbefore defined,

a C₄₋₆-cycloalkoxy or C₃₋₆-cycloalkyl-C₁₋₃-alkoxy group,

C denotes an —O—C₁₋₄-alkylene or —O—CH₂—CH(OH)—CH₂ group, wherein the oxygen atom of the abovementioned groups in each case is linked to the bicyclic heteroaromatic ring,

D denotes an R₆O—CO-alkylene-NR₅ group wherein the alkylene moiety, which is straight-chained and contains 1 or 2 carbon atoms, may additionally be substituted by an R₆O—CO or R₆O—CO-methyl group, wherein R₅ and R₆ are as hereinbefore defined,

a pyrrolidino or piperidino group which is substituted by an R₆O—CO or R₆O—CO—C₁₋₂-alkyl group wherein R₆ is as hereinbefore defined,

a pyrrolidino or piperidino group which is substituted by two R₆O—CO or R₆O—CO—C₁₋₂-alkyl groups wherein R₆ is as hereinbefore defined,

a piperazino group which is substituted in the 4 position by the group R₁₀ and additionally at a cyclic carbon atom by an R₆O—CO or R₆O—CO—C₁₋₂-alkyl group wherein R₆ and R₁₀ are as hereinbefore defined,
a piperazino group which is substituted in the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ to R₁₀ are as hereinbefore defined,

a piperazino group which is substituted in the 4 position by an R₆O—CO—C₁₋₂-alkyl group and additionally at a cyclic carbon atom by an R₆O—CO or R₆O—CO—C₁₋₂-alkyl group wherein R₆ is as hereinbefore defined,

a morpholino group which is substituted by an R₆O—CO or R₆O—CO—C₁₋₂-alkyl group, wherein R₆ is as hereinbefore defined,

a pyrrolidinyl or piperidinyl group substituted in the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ to R₉ are as hereinbefore defined,

a 2-oxomorpholino group which may be substituted by 1 or 2 methyl groups,

a 2-oxomorpholinyl group which is substituted in the 4 position by a methyl, ethyl, or R₆O—CO—C₁₋₂-alkyl group, wherein R₆ is as hereinbefore defined and the abovementioned 2-oxomorpholinyl groups are in each case linked to a carbon atom of the group C,

a R₁₁N(C₁₋₂-alkyl) group wherein R₁₁ denotes a 2-oxotetrahydrofuran-3-yl or 2-oxotetrahydrofuran-4-yl group, or

C and D together denote a hydrogen atom, or a methoxy, ethoxy, or 2-methoxyethoxy group,

a C₁₋₂-alkoxy group which is substituted by an R₆O—CO group, wherein R₆ is as hereinbefore defined,

a C₄₋₆-cycloalkoxy or C₃₋₆-cycloalkyl-C₁₋₃-alkoxy group

with the proviso that at least one of the groups B or D, or A together with B, or C together with D contains an optionally substituted 2-oxomorpholinyl group, a (R₇O—PO—OR₈) or (R₇O—PO—R₉) group, or

that at least one of the groups A, B, C, or D, or A together with B, or C together with D contains an R₆O—CO group and additionally one of the groups A, B, C, or D, or A together with B, or C together with D contains a primary, secondary, or tertiary amino function, wherein the nitrogen atom of this amino function is not linked to a carbon atom of an aromatic group, the tautomers, stereoisomers, and salts thereof.

Most particularly preferred compounds of general formula I are those wherein:

Rₐ denotes a hydrogen atom,

R_{b} denotes a phenyl, benzyl, or 1-phenylethyl group wherein the phenyl nucleus is substituted in each case by the groups R₁ to R₃, wherein:
R₁ and R₂, which may be identical or different, each denote a hydrogen, fluorine, chlorine, or bromine atom,
a methyl, trifluoromethyl, methoxy, ethynyl, or cyano group, and
R₃ denotes a hydrogen atom,

R_{c} and R_{d} in each case denote a hydrogen atom,

X denotes a nitrogen atom,

A denotes an —O—C₁₋₄-alkylene or —O—CH₂—CH(OH)—CH₂ group, wherein the oxygen atom of the abovementioned groups in each case is linked to the bicyclic heteroaromatic ring,

B denotes an R₆O—CO-alkylene-NR₅ group wherein the alkylene moiety, which is straight-chained and contains 1 or 2 carbon atoms, may additionally be substituted by an R₆O—CO or R₆O—CO-methyl group, wherein:
R₅ denotes a hydrogen atom,
a C₁₋₂-alkyl group which may be substituted by an R₆O—CO group,
a C₂₋₄-alkyl group which is substituted from position 2 onwards by a hydroxy group,
a C₃₋₆-cycloalkyl or C₃₋₆-cycloalkylmethyl group, and
R₆ denotes a hydrogen atom,
a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group, wherein:
   Rₑ denotes a hydrogen atom or a C₁₋₄-alkyl group,
   R_{f} denotes a hydrogen atom, and
   R_{g} denotes a C₁₋₄-alkyl, cyclopentyl, cyclohexyl, C₁₋₄-alkoxy, cyclopentyloxy, or cyclohexyloxy group,

a pyrrolidino or piperidino group which is substituted by an R₆O—CO or R₆O—CO—C₁₋₂-alkyl group wherein R₆ is as hereinbefore defined,

a pyrrolidino or piperidino group which is substituted by two R₆O—CO or R₆O—CO—C₁₋₂-alkyl groups wherein R₆ is as hereinbefore defined,

a piperazino group which is substituted in the 4 position by the group R₁₀ and additionally at a cyclic carbon atom by an R₆O—CO or R₆O—CO—C₁₋₂-alkyl group wherein R₆ is as hereinbefore defined, and
R₁₀ denotes a hydrogen atom, or a methyl or ethyl group,

a piperazino group which is substituted in the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ is as hereinbefore defined,
R₇ and R₈ which may be identical or different, in each case denote a hydrogen atom, or a methyl, ethyl, phenyl, benzyl, 5-indanyl, or R_{g}CO—O—(RₑCR_{f}) group, wherein:
   Rₑ to R_{g} are as hereinbefore defined,
and R₉ denotes a methyl or ethyl group,

a piperazino group which is substituted in the 4 position by an R₆O—CO—C₁₋₂-alkyl group and is additionally substituted at a cyclic carbon atom by an R₆O—CO or R₆O—CO—C₁₋₂-alkyl group wherein R₆ is as hereinbefore defined,

a morpholino group which is substituted by an R₆O—CO or R₆O—CO—C₁₋₂-alkyl group, wherein R₆ is as hereinbefore defined,

a pyrrolidinyl or piperidinyl group substituted in the 1 position by an R₆O—CO—C₁₋₂-alkyl, bis-(R₆O—CO)—C₁₋₄-alkyl, (R₇O—PO—OR₈)-methyl, or (R₇O—PO—R₉)-methyl group wherein R₆ to R₉ are as hereinbefore defined,

a 2-oxomorpholino group which may be substituted by 1 or 2 methyl groups,

a 2-oxomorpholinyl group which is substituted in the 4 position by a methyl, ethyl, or R₆O—CO—C₁₋₂-alkyl group, wherein R₆ is as hereinbefore defined and the abovementioned 2-oxomorpholinyl groups in each case are linked to a carbon atom of the group A,

a R₁₁N(C₁₋₂-alkyl) group wherein R₁₁ denotes a 2-oxotetrahydrofuran-3-yl or 2-oxotetrahydrofuran-4-yl group, and

C and D together denote a hydrogen atom, or a methoxy, ethoxy, 2-methoxyethoxy, C₄₋₆-cycloalkoxy, or C₃₋₆-cycloalkyl-C₁₋₃-alkoxy group,

particularly those compounds wherein:

Rₐ denotes a hydrogen atom,

R_{b} denotes a phenyl, benzyl, or 1-phenylethyl group wherein the phenyl nucleus is substituted in each case by the groups R₁ to R₃, wherein:
R₁ and R₂, which may be identical or different, each denote a hydrogen, fluorine, chlorine, or bromine atom,
a methyl, trifluoromethyl, methoxy, ethynyl, or cyano group, and
R₃ denotes a hydrogen atom,

R_{c} and R_{d} in each case denote a hydrogen atom,

X denotes a nitrogen atom,

A and B together denote a hydrogen atom, or a methoxy, ethoxy, 2-methoxyethoxy, C₄₋₆-cycloalkoxy, or C₃₋₆-cycloalkyl-C₁₋₃-alkoxy group,

C denotes an —O—C₁₋₄-alkylene or —O—CH₂—CH(OH)—CH₂ group, wherein the oxygen atom of the abovementioned groups in each case is linked to the bicyclic heteroaromatic ring, and

D denotes an R₆O—CO-alkylene-NR₅ group wherein the alkylene moiety, which is straight-chained and contains 1 or 2 carbon atoms, may additionally be substituted by an R₆O—CO or R₆O—CO-methyl group, wherein:
R₅ denotes a hydrogen atom,
a C₁₋₂-alkyl group which may be substituted by an R₆O—CO group,
a C₂₋₄-alkyl group which is substituted from position 2 by a hydroxy group,
a C₃₋₆-cycloalkyl or C₃₋₆-cycloalkylmethyl group, and
R₆ denotes a hydrogen atom,
a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group, wherein:
   Rₑ denotes a hydrogen atom or a C₁₋₄-alkyl group,
   R_{f} denotes a hydrogen atom, and
   R_{g} denotes a C₁₋₄-alkyl, cyclopentyl, cyclohexyl, C₁₋₄-alkoxy, cyclopentyloxy, or cyclohexyloxy group,

a pyrrolidino or piperidino group which is substituted by an R₆O—CO or R₆O—CO—C₁₋₂-alkyl group wherein R₆ is as hereinbefore defined,

a pyrrolidino or piperidino group which is substituted by two R₆O—CO or R₆O—CO—C₁₋₂-alkyl groups wherein R₆ is as hereinbefore defined,

a piperazino group which is substituted in the 4 position by the group R₁₀ and additionally at a cyclic carbon atom by an R₆O—CO or R₆O—CO—C₁₋₂-alkyl group wherein R₆ is as hereinbefore defined, and
R₁₀ denotes a hydrogen atom, or a methyl or ethyl group,

a piperazino group which is substituted in the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ is as hereinbefore defined,
R₇ and R₈, which may be identical or different, in each case denote a hydrogen atom, or a methyl, ethyl, phenyl, benzyl, 5-indanyl, or R₉CO—O—(RₑCR_{f}) group, wherein:
   Rₑ to R_{g} are as hereinbefore defined,
and R₉ denotes a methyl or ethyl group,

a piperazino group which is substituted in the 4 position by an R₆O—CO—C₁₋₂-alkyl group and is additionally substituted at a cyclic carbon atom by an R₆O—CO or R₆O—CO—C₁₋₂-alkyl group wherein R₆ is as hereinbefore defined,

a morpholino group which is substituted by an R₆O—CO or R₆O—CO—C₁₋₂-alkyl group, wherein R₆ is as hereinbefore defined,

a pyrrolidinyl or piperidinyl group substituted in the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group wherein R₆ to R₉ are as hereinbefore defined,

a 2-oxomorpholino group which may be substituted by 1 or 2 methyl groups,

a 2-oxomorpholinyl group which is substituted in the 4 position by a methyl, ethyl, or R₆O—CO—C₁₋₂-alkyl group, wherein R₆ is as hereinbefore defined and the abovementioned 2-oxomorpholinyl groups are in each case linked to a carbon atom of the group C, or

a R₁₁N(C₁₋₂-alkyl) group wherein R₁₁ denotes a 2-oxotetrahydrofuran-3-yl or 2-oxotetrahydrofuran-4-yl group,

the tautomers, stereoisomers, and salts thereof.

The most preferred bicyclic heterocyclic compounds of general formula I, however, are those wherein:

Rₐ denotes a hydrogen atom,

R_{b} denotes a phenyl group wherein the phenyl nucleus is substituted in each case by the groups R₁ to R₃, wherein:
R₁ and R₂, which may be identical or different, each denote a hydrogen, fluorine, chlorine, or bromine atom, and
R₃ denotes a hydrogen atom,

R_{c} and R_{d} in each case denote a hydrogen atom,

X denotes a nitrogen atom,

A denotes an —O—C₁₋₄-alkylene or —O—CH₂—CH(OH)—CH₂ group, wherein the oxygen atom of the abovementioned groups in each case is linked to the bicyclic heteroaromatic ring,

B denotes an R₆O—CO—CH₂—NR₅ group wherein:
R₅ denotes a hydrogen atom or a methyl group which may be substituted by an R₆O—CO group, or
a C₂₋₄-alkyl group substituted from position 2 onwards by a hydroxy group, and
R₆ denotes a hydrogen atom, or a methyl or ethyl group,

a pyrrolidino or piperidino group which is substituted by an R₆O—CO group, wherein R₆ is as hereinbefore defined,

a piperazino group which is substituted in the 4 position by an R₆O—CO—CH₂ or bis-(R₆O—CO)—C₁₋₃-alkyl group, wherein R₆ is as hereinbefore defined,

a pyrrolidinyl or piperidinyl group substituted in the 1 position by an R₆O—CO—CH₂ group, wherein R₆ is as hereinbefore defined,

a 2-oxomorpholino group which may be substituted by one or two methyl groups, or

a R₁₁N(C₁₋₂-alkyl) group wherein R₁₁ denotes a 2-oxotetrahydrofuran-3-yl or 2-oxotetrahydrofuran-4-yl group, and

C and D together denote a methoxy, C₄₋₆-cycloalkoxy, or C₃₋₆-cycloalkylmethoxy group,

particularly those compounds wherein:

Rₐ denotes a hydrogen atom,

R_{b} denotes a phenyl group wherein the phenyl nucleus is substituted in each case by the groups R₁ to R₃, wherein:
R₁ and R₂, which may be identical or different, each denote a hydrogen, fluorine, chlorine, or bromine atom, and
R₃ denotes a hydrogen atom,

R_{c} and R_{d} in each case denote a hydrogen atom,

X denotes a nitrogen atom,

A and B together denote a C₄₋₆-cycloalkoxy or C₃₋₆-cycloalkylmethoxy group,

C denotes an —O—CH₂CH₂ group, wherein the oxygen atom of the abovementioned group is linked to the bicyclic heteroaromatic ring,

D denotes an R₆O—CO—CH₂—NR₅ group wherein:
R₅ denotes a C₂₋₄-alkyl group substituted from position 2 onwards by a hydroxy group, and
R₆ denotes a methyl or ethyl group,

a 2-oxomorpholino group which may be substituted by one or two methyl groups, or

a R₁₁N(C₁₋₂-alkyl) group wherein R₁₁ denotes a 2-oxotetrahydrofuran-3-yl or 2-oxotetrahydrofuran-4-yl group,

the tautomers, stereoisomers, and salts thereof.

The following particularly preferred compounds of general formula I are mentioned by way of example:
(1) 4-(3-chloro-4-fluorophenylamino)-6-{3-[4-(methoxycarbonylmethyl)-1-piperazinyl]propyloxy}-7-methoxyquinazoline,
(2) 4-[(3-bromophenyl)amino]-6-(2-{4-[(ethoxycarbonyl)methyl]piperazin-1-yl}ethoxy)-7-methoxyquinazoline;
(3) (S)-4-[(3-bromophenyl)amino]-6-[3-(2-methoxycarbonylpyrrolidin-1-yl)propyloxy]-7-methoxyquinazoline;
(4) 4-[(3-bromophenyl)amino]-6-(3-{4-[(ethoxycarbonyl)methyl]piperazin-1-yl}-2-hydroxypropyloxy)-7-methoxyquinazoline;
(5) (S)-4-[(3-bromophenyl)amino]-6-({1-[(ethoxycarbonyl)methyl]pyrrolidine-2-yl}methoxy)-7-methoxyquinazoline; and
(6) 4-[(3-bromophenyl)amino]-6-(2-{4-[1,2-bis(methoxycarbonyl)ethyl]piperazin-1-yl}ethoxy)-7-methoxyquinazoline,

and the salts thereof.

### DETAILED DESCRIPTION OF THE INVENTION

The compounds of general formula I may, for example, be prepared by the following methods:

(a) reacting a compound of general formula

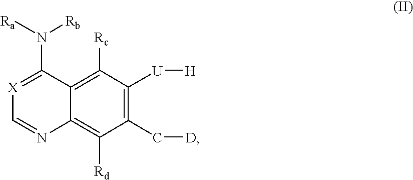

wherein:

Rₐ to R_{d}, C, D, and X are as hereinbefore defined, and

U denotes an oxygen atom or an R₄N group, wherein R₄ is as hereinbefore defined, with a compound of general formula
Z_{1-A′-B} (III)

wherein:

B is as hereinbefore defined,

A′denotes one of the optionally substituted alkylene, cycloalkylene, alkylene-cycloalkylene, cycloalkylene-alkylene, or alkylene-cycloalkylene-alkylene moieties mentioned above for the group A, which are linked to the heteroaromatic group via an oxygen atom or via an NR₄ group, and

Z₁ denotes a leaving group such as a halogen atom or a sulfonyloxy group such as a chlorine or bromine atom, or a methanesulfonyloxy or p-toluenesulfonyloxy group.

The reaction is optionally carried out in a solvent or mixture of solvents such as methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine, pyridine, or 2-dimethylaminopyridine, in the presence of N-ethyldiisopropylamine (Hünig's base), wherein these organic bases may simultaneously serve as solvents, or in the presence of an inorganic base such as sodium carbonate, potassium carbonate, or sodium hydroxide solution conveniently at temperatures between −20° C. and 200° C., preferably at temperatures between 0° C. and 150° C.

b) reacting a compound of general formula

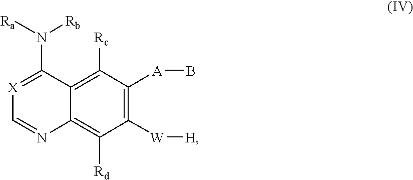

wherein:

Rₐ to R_{d}, A, B, and X are as hereinbefore defined; and

W denotes an oxygen atom or an R₄N group, wherein R₄ is as hereinbefore defined, with a compound of general formula
Z₂-C′-D (V)

wherein:

D is as hereinbefore defined,

C′ denotes one of the optionally substituted alkylene, cycloalkylene, alkylene-cycloalkylene, cycloalkylene-alkylene, or alkylene-cycloalkylene-alkylene moieties mentioned above for the group C, which are linked to the heteroaromatic group via an oxygen atom or via an NR₄ group, and

Z₂ denotes a leaving group such as a halogen atom or a sulfonyloxy group such as a chlorine or bromine atom, or a methanesulfonyloxy or p-toluenesulfonyloxy group.

The reaction is optionally carried out in a solvent or mixture of solvents such as methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine, pyridine, or 2-dimethylaminopyridine, in the presence of N-ethyoxyetiisopropylamine (Hünig's base), wherein these organic bases may simultaneously serve as solvents, or in the presence of an inorganic base such as sodium carbonate, potassium carbonate, or sodium hydroxide solution, or in the presence of an alkali or alkaline earth metal alkoxide such as sodium ethoxide or potassium tert-butoxide conveniently at temperatures between −20° C. and 200° C., preferably at temperatures between 0° C. and 150° C.

c) In order to prepare a compound of general formula I wherein A is as hereinbefore defined with the exception of the oxygen atom and the —NR₄ group:

reacting a compound of general formula

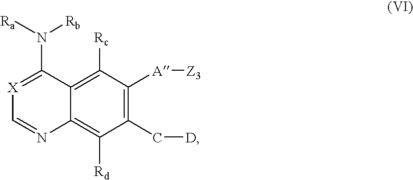

wherein:

Rₐ to R_{d}, C, D, and X are as hereinbefore defined, and

A″ has the meanings given for A hereinbefore with the exception of the oxygen atom and the —NR₄ group, and

Z₃ denotes a leaving group such as a halogen atom or a sulfonyloxy group such as a chlorine or bromine atom, or a methanesulfonyloxy or p-toluenesulfonyloxy group, or together with a hydrogen atom of an adjacent hydrocarbon group denotes an oxygen atom, with a compound of general formula
H—B (VII)

wherein B is as hereinbefore defined.

The reaction is optionally carried out in a solvent or mixture of solvents such as acetonitrile, ethanol, methylene chloride, dimethylformamide, dimethylsulfoxide, sulfolane, benzene, toluene, chlorobenzene, tetrahydrofuran, benzene/tetrahydrofuran, or dioxane, optionally in the presence of a tertiary organic base such as triethylamine, pyridine, or 2-dimethyl-aminopyridine, in the presence of N-ethyldiisopropylamine (Hünig's base), wherein these organic bases may simultaneously serve as solvents, or in the presence of an inorganic base such as sodium carbonate, potassium carbonate, or sodium hydroxide solution, or in the presence of an alkali or alkaline earth metal alkoxide such as sodium ethoxide or potassium tert-butoxide, conveniently at temperatures between −20° C. and 200° C., preferably at temperatures between 0° C. and 150° C.

d) In order to prepare a compound of general formula I wherein C is as hereinbefore defined with the exception of the oxygen atom and the —NR₄ group:

reacting a compound of general formula

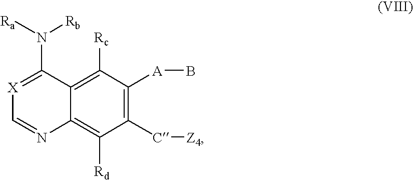

wherein:

C″ has the meanings given for C hereinbefore with the exception of the oxygen atom and the —NR₄ group, and

Z₄ denotes a leaving group such as a halogen atom or a sulfonyloxy group such as a chlorine or bromine atom, or a methanesulfonyloxy or p-toluenesulfonyloxy group, or together with a hydrogen atom of an adjacent hydrocarbon group denotes an oxygen atom, with a compound of general formula
H-D (IX)

wherein D is as hereinbefore defined.

The reaction is optionally carried out in a solvent or mixture of solvents such as acetonitrile, ethanol, methylene chloride, dimethylformamide, dimethylsulfoxide, sulfolane, benzene, toluene, chlorobenzene, tetrahydrofuran, benzene/tetrahydrofuran, or dioxane optionally in the presence of a tertiary organic base such as triethylamine, pyridine, or 2-dimethyl-aminopyridine, in the presence of N-ethyldiisopropylamine (Hünig's base), wherein these organic bases may simultaneously serve as solvents, or in the presence of an inorganic base such as sodium carbonate, potassium carbonate, or sodium hydroxide solution, or in the presence of an alkali or alkaline earth metal alkoxide such as sodium ethoxide or potassium tert-butoxide, conveniently at temperatures between −20° C. and 200° C., preferably at temperatures between 0° C. and 150° C.

e) In order to prepare a compound of general formula I wherein B denotes an R₆O—CO-alkylene-NR₅ group wherein the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, a piperazino or homopiperazino group substituted in the 4 position by an R₆O—CO—C₁₋₄-alkyl or bis-(R₆O—CO)—C₁₋₄-alkyl group or a pyrrolidinyl, piperidinyl, or hexahydroazepinyl group substituted in the 1 position by an R₆O—CO—C₁₋₄-alkyl or bis-(R₆O—CO)—C₁₋₄-alkyl group, wherein in each case R₅ and R₆ are as hereinbefore defined:

reacting a compound of general formula

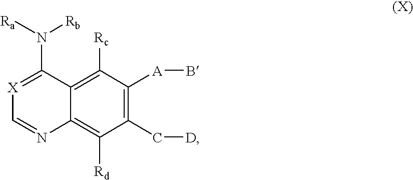

wherein:

Rₐ to R_{d}, A, C, D, and X are as hereinbefore defined, and

B′ denotes an R₅NH group wherein R₅ is as hereinbefore defined, a piperazino or homopiperazino group unsubstituted in the 4 position, a pyrrolidinyl, piperidinyl, or hexahydroazepinyl group unsubstituted in the 1 position, with a compound of general formula
R₆O—CO-alkylene-Z₅ (XI)

wherein:

the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, wherein R₆ in each case is as hereinbefore defined, and

Z₅ denotes an exchangeable group such as a halogen atom or a substituted sulfonyloxy group, e.g., a chlorine or bromine atom, or a methylsulfonyloxy, propylsulfonyloxy, phenylsulfonyloxy, or benzylsulfonyloxy group.

The reaction is optionally carried out in a solvent or mixture of solvents such as acetonitrile, methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine or N-ethyldiisopropylamine (Hünig's base), wherein these organic bases may simultaneously serve as solvents, or in the presence of an inorganic base such as sodium carbonate, potassium carbonate or sodium hydroxide solution conveniently at temperatures between −20° C. and 200° C., preferably at temperatures between 0° C. and 150° C.

f) In order to prepare a compound of general formula I wherein D denotes an R₆O—CO-alkylene-NR₅ group wherein the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, a piperazino or homopiperazino group substituted in the 4 position by an R₆O—CO—C₁₋₄-alkyl or bis-(R₆O—CO)—C₁₋₄-alkyl group or a pyrrolidinyl, piperidinyl, or hexahydroazepinyl group substituted in the 1 position by an R₆O—CO—C₁₋₄-alkyl or bis-(R₆O—CO)—C₁₋₄-alkyl group, wherein in each case R₅ and R₆ are as hereinbefore defined:

reacting a compound of general formula

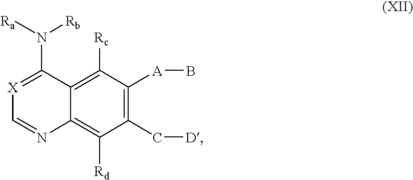

wherein:

Rₐ to R_{d}, A to C, and X are as hereinbefore defined, and

D′ denotes an R₅NH group wherein R₅ is as hereinbefore defined, a piperazino or homopiperazino group unsubstituted in the 4 position, a pyrrolidinyl, piperidinyl, or hexahydroazepinyl group unsubstituted in the 1 position, with a compound of general formula
R₆O—CO-alkylene-Z₅ (XI)

wherein:

the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, wherein R₆ in each case is as hereinbefore defined, and

Z₅ denotes an exchangeable group such as a halogen atom or a substituted sulfonyloxy group, e.g., a chlorine or bromine atom, or a methylsulfonyloxy, propylsulfonyloxy, phenylsulfonyloxy, or benzylsulfonyloxy group.

The reaction is optionally carried out in a solvent or mixture of solvents such as acetonitrile, methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine or N-ethyldiisopropylamine (Hünig's base), wherein these organic bases may simultaneously serve as solvents, or in the presence of an inorganic base such as sodium carbonate, potassium carbonate, or sodium hydroxide solution conveniently at temperatures between −20° C. and 200° C., preferably at temperatures between 0° C. and 150° C.

g) In order to prepare a compound of general formula I wherein at least one of the groups R₆ to R₈ denotes a hydrogen atom:

Converting a compound of general formula

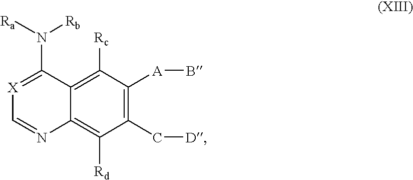

wherein:

Rₐ to R_{d}, A, C, and X are as hereinbefore defined,

B″ and D″ have the meanings given for B and D hereinbefore, with the proviso that at least one of the groups B″ or D″ contains an R₆O—CO, (R₇O—PO—OR₈), or (R₇O—PO—R₉) group wherein

R₉ is as hereinbefore defined and at least one of the groups R₆ to R₈ does not represent a hydrogen atom, by hydrolysis, treating with acids, thermolysis, or hydrogenolysis, into a compound of general formula I wherein at least one of the groups R₆ to R₈ denotes a hydrogen atom.

The hydrolysis is conveniently carried out either in the presence of an acid such as hydrochloric acid, sulfuric acid, phosphoric acid, acetic acid, trichloroacetic acid, trifluoroacetic acid or mixtures thereof, or in the presence of a base such as lithium hydroxide, sodium hydroxide, or potassium hydroxide in a suitable solvent such as water, water/methanol, water/ethanol, water/isopropanol, methanol, ethanol, water/tetrahydrofuran, or water/dioxane at temperatures between −10° C. and 120° C., e.g., at temperatures between ambient temperature and the boiling temperature of the reaction mixture.

If B″ or D″ in a compound of formula X, for example, contains the tert-butyloxycarbonyl group, the tert-butyl group may also be cleaved by treating with an acid such as trifluoroacetic acid, formic acid, p-toluenesulfonic acid, sulfuric acid, hydrochloric acid, phosphoric acid, or polyphosphoric acid optionally in an inert solvent such as methylene chloride, chloroform, benzene, toluene, diethylether, tetrahydrofuran, or dioxane preferably at temperatures between —10° C. and 120° C., e.g., at temperatures between 0° C. and 60° C., or thermally, optionally in an inert solvent such as methylene chloride, chloroform, benzene, toluene, tetrahydrofuran, or dioxane and preferably in the presence of a catalytic amount of an acid such as p-toluenesulfonic acid, sulfuric acid, phosphoric acid, or polyphosphoric acid preferably at the boiling temperature of the solvent used, e.g., at temperatures between 40° C. and 120° C. Under the reaction conditions mentioned above, any N-tert-butyloxycarbonylamino or N-tert-butyloxycarbonylimino groups present may be converted into the corresponding amino or imino groups.

If B″ or D″ in a compound of formula X, for example, contains the benzyloxycarbonyl group, the benzyl group may also be cleaved hydrogenolytically in the presence of a hydrogenation catalyst such as palladium/charcoal in a suitable solvent such as methanol, ethanol, ethanol/water, glacial acetic acid, ethyl acetate, dioxane, or dimethylformamide, preferably at temperatures between 0° C. and 50° C., e.g., ambient temperature, and at a hydrogen pressure of 1 to 5 bar. During the hydrogenolysis other groups may simultaneously be converted, e.g., a nitro group may be converted into an amino group, a benzyloxy group into a hydroxy group and a N-benzylamino, N-benzylimino, N-benzyloxycarbonylamino, or N-benzyloxycarbonylimino group into a corresponding amino or imino group.

If according to the invention a compound of general formula I is obtained which contains a carboxy or hydroxyphosphoryl group, this may be converted by esterification into a corresponding ester of general formula I or

If a compound of general formula I is obtained wherein B or D denotes an optionally substituted N-(2-hydroxyethyl)glycine or N-(2-hydroxyethyl)glycinester group, this may be converted by cyclization in a corresponding 2-oxomorpholino compound.

The subsequent esterification is optionally carried out in a solvent or mixture of solvents such as methylene chloride, dimethylformamide, benzene, toluene, chlorobenzene, tetrahydrofuran, benzene/tetrahydrofuran, or dioxane or particularly advantageously in a corresponding alcohol, optionally in the presence of an acid such as hydrochloric acid, or in the presence of a dehydrating agent, e.g., in the presence of isobutyl chloroformate, thionyl chloride, trimethylchlorosilane, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, or 1-hydroxybenzotriazole and optionally additionally in the presence of 4-dimethylaminopyridine, N,N′-carbonyldiimidazole, or triphenylphosphine/carbon tetrachloride, conveniently at temperatures between 0° C. and 150° C., preferably at temperatures between 0° C. and 80° C.

The subsequent ester formation may also be carried out by reacting a compound which contains a carboxy or hydroxyphosphoryl group with a corresponding alkyl halide.

The subsequent intramolecular cyclization is optionally carried out in a solvent or mixture of solvents such as acetonitrile, methylene chloride, tetrahydrofuran, dioxane, or toluene in the presence an acid such as hydrochloric acid or p-toluenesulfonic acid at temperatures between −10° C. and 120° C.

In the reactions described hereinbefore, any reactive groups present such as hydroxy, carboxy, phosphono, O-alkyl-phosphono, amino, alkylamino, or imino groups may be protected during the reaction by conventional protecting groups which are cleaved again after the reaction.

For example, a protecting group for a hydroxy group may be a trimethylsilyl, acetyl, benzoyl, methyl, ethyl, tert-butyl, trityl, benzyl, or tetrahydropyranyl group,

protecting groups for a carboxy group may be a trimethylsilyl, methyl, ethyl, tert-butyl, benzyl, or tetrahydropyranyl group,

protecting groups for a phosphono group may be an alkyl group such as a methyl, ethyl, isopropyl, or n-butyl group, or a phenyl or benzyl group, and

protecting groups for an amino, alkylamino, or imino group may be a formyl, acetyl, trifluoroacetyl, ethoxycarbonyl, tert-butoxycarbonyl, benzyloxycarbonyl, benzyl, methoxybenzyl, or 2,4-dimethoxybenzyl group and additionally, for the amino group, a phthalyl group.

Any protecting group used is optionally subsequently cleaved for example by hydrolysis in an aqueous solvent, e.g., in water, isopropanol/water, acetic acid/water, tetrahydrofuran/water, or dioxane/water, in the presence of an acid such as trifluoroacetic acid, hydrochloric acid, or sulfuric acid, or in the presence of an alkali metal base such as sodium hydroxide or potassium hydroxide or aprotically, e.g., in the presence of iodotrimethylsilane, at temperatures between 0° C. and 120° C., preferably at temperatures between 10° C. and 100° C.

However, a benzyl, methoxybenzyl, or benzyloxycarbonyl group is cleaved, for example, hydrogenolytically, e.g., with hydrogen in the presence of a catalyst such as palladium/charcoal in a suitable solvent such as methanol, ethanol, ethyl acetate, or glacial acetic acid, optionally with the addition of an acid such as hydrochloric acid at temperatures between 0° C. and 100° C., but preferably at temperatures between 20° C. and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert-butyl or tert-butyloxycarbonyl group is preferably cleaved by treating with an acid such as trifluoroacetic acid or hydrochloric acid or by treating with iodotrimethylsilane, optionally using a solvent such as methylene chloride, dioxane, methanol, or diethylether.

A trifluoroacetyl group is preferably cleaved by treating with an acid such as hydrochloric acid, optionally in the presence of a solvent such as acetic acid at temperatures between 50° C. and 120° C. or by treating with sodium hydroxide solution optionally in the presence of a solvent such as tetrahydrofuran at temperatures between 0° C. and 50° C.

A phthalyl group is preferably cleaved in the presence of hydrazine or a primary amine such as methylamine, ethylamine, or n-butylamine in a solvent such as methanol, ethanol, isopropanol, toluene/water, or dioxane at temperatures between 20° C. and 50° C.

A single alkyl group may be cleaved from an O,O′-dialkylphosphono group with sodium iodide, for example, in a solvent such as acetone, methyl ethyl ketone, acetonitrile, or dimethylformamide at temperatures between 40° C. and 150° C., but preferably at temperatures between 60° C. and 100° C.

Both alkyl groups may be cleaved from an O,O′-dialkylphosphono group with iodotrimethylsilane, bromotrimethylsilane, or chlorotrimethylsilane/sodium iodide, for example, in a solvent such as methylene chloride, chloroform, or acetonitrile at temperatures between 0° C. and the boiling temperature of the reaction mixture, but preferably at temperatures between 20 and 60° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. N. L. Allinger and E. L. Eliel in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.

The enantiomers are preferably separated by column separation on chiral phases or by recrystallization from an optically active solvent or by reacting with an optically active substance which forms salts or derivatives such as e.g., esters or amides with the racemic compound, particularly acids and the activated derivatives or alcohols thereof, and separating the diastereomeric mixture of salts or derivatives thus obtained, e.g., on the basis of their differences in solubility, wherein the free antipodes may be released from the pure diastereomeric salts or derivatives by the action of suitable agents. Optically active acids in common use are e.g., the D- and L-forms of tartaric acid or dibenzoyltartaric acid, di-o-tolyltartaric acid, malic acid, mandelic acid, camphorsulfonic acid, glutamic acid, aspartic acid, or quinic acid. An optically active alcohol may be for example (+) or (−)-menthol and an optically active acyl group in amides, for example, may be a (+)-or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulfuric acid, phosphoric acid, fumaric acid, succinic acid, lactic acid, citric acid, tartaric acid, or maleic acid.

Moreover, if the new compounds of formula I thus obtained contain a carboxy, hydroxyphosphoryl, sulfo, or 5-tetrazolyl group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine, and triethanolamine.

The compounds of general formulae II to XIII used as starting materials are known from the literature in some cases or may be obtained by methods known from the literature (cf. Examples I to XVI).

As already mentioned hereinbefore, the compounds of general formula I according to the invention and their physiologically acceptable salts have valuable pharmacological properties, particularly an inhibiting effect on signal transduction mediated by the Epidermal Growth Factor receptor (EGF-R), wherein this may be achieved for example by inhibiting ligand bonding, receptor dimerization or tyrosine kinase itself. It is also possible to block the transmission of signals to components located further down.

The biological properties of the new compounds were investigated as follows. The inhibition of the EGF-R-mediated signal transmission can be demonstrated e.g., with cells which express human EGF-R and whose survival and proliferation depend on stimulation by EGF or TGF-alpha. A cell line of murine origin dependent on interleukin-3-(IL-3) which was genetically modified to express functional human EGF-R was used here. The proliferation of these cells known as F/L-HERc can therefore be stimulated either by murine IL-3 or by EGF (cf. T. von Rüden et al. in EMBO J. 7, 2749-2756 (1988) and J. H. Pierce et al. in Science 239, 628-631 (1988)). The starting material used for the F/L-HERc cells was the cell line FDC-P₁, the production of which has been described by T. M. Dexter et al. in J. Exp. Med. 152, 1036-1047 (1980). Alternatively, however, other growth-factor-dependent cells may also be used (cf., for example, J. H. Pierce et al. in Science 239, 628-631 (1988); H. Shibuya et al. in Cell 70, 57-67 (1992) and W. S. Alexander in EMBO J. 10, 3683-3691 (1991)). For expressing the human EGF-R cDNA (cf. A. Ullrich et al. in Nature 309, 418-425 (1984)) recombinant retroviruses were used as described by T. von Rüden et al., EMBO J. 7, 2749-2756 (1988), except that the retroviral vector LXSN (cf. A. D. Miller et al. in BioTechniques 7, 980-990 (1989)) was used for the expression of the EGF-R cDNA and the line GP+E86 (cf. D. Markowitz et al. in J. Virol. 62, 1120-1124 (1988)) was used as the packaging cell.

The test was performed as follows. F/L-HERc cells were cultivated in RPMI/1640 medium (BioWhittaker), supplemented with 10% fetal calf serum (FCS, Boehringer Mannheim), 2 mM glutamine (BioWhittaker), standard antibiotics and 20 ng/ml of human EGF (Promega), at 37° C. and 5% CO₂. In order to investigate the inhibitory activity of the compounds according to the invention, 1.5×10⁴ cells per well were cultivated in triplicate in 96-well dishes in the above medium (200 μl), the cell proliferation being stimulated with either EGF (20 ng/ml) or murine IL-3. The IL-3 used was obtained from culture supernatants of the cell line X63/0 mIL-3 (cf. H. Karasuyama et al. in Eur. J. Immunol. 18, 97-104 (1988)). The compounds according to the invention were dissolved in 100% dimethylsulfoxide (DMSO) and added to the cultures in various dilutions, the maximum DMSO concentration being 1%. The cultures were incubated for 48 hours at 37° C.

In order to determine the inhibitory activity of the compounds according to the invention the relative cell number was measured in O.D. units using the Cell Titer 96™ Aqueous Non-Radioactive Cell Proliferation Assay (Promega). The relative cell number was calculated as a percentage of the control (F/LHERc cells without inhibitor) and the concentration of active substance which inhibits the proliferation of the cells by 50% (IC₅₀) was derived therefrom. The following results were obtained:

| Compound | Inhibition of EGF-Dependent |
|---|---|
| (Example no) | Proliferation IC₅₀ [nM] |
| 1 | 46 |
| 1(2) | 20 |
| 2 | 230 |
| 2(1) | 39 |
| 3 | 45 |
| 3(1) | 100 |
| 3(2) | 70 |
| 3(4) | 77 |
| 4 | 33 |

The compounds of general formula I according to the invention thus inhibit the signal transduction by tyrosine kinases, as demonstrated by the example of the human EGF receptor, and are therefore useful for treating pathophysiological processes caused by hyperfunction of tyrosine kinases. These are e.g., benign or malignant tumors, particularly tumors of epithelial and neuroepithelial origin, metastasization, and the abnormal proliferation of vascular endothelial cells (neoangiogenesis).

The compounds according to the invention are also useful for preventing and treating diseases of the airways and lungs which are accompanied by increased or altered production of mucus caused by stimulation by tyrosine kinases, e.g., in inflammatory diseases of the airways such as chronic bronchitis, chronic obstructive bronchitis, asthma, bronchiectasias, allergic or non-allergic rhinitis or sinusitis, cystic fibrosis, α1-antitrypsin deficiency, or coughs, pulmonary emphysema, pulmonary fibrosis, and hyperreactive airways.

The compounds are also suitable for treating diseases of the gastrointestinal tract and bile duct and gall bladder which are associated with disrupted activity of the tyrosine kinases, such as may be found e.g., in chronic inflammatory changes such as cholecystitis, Crohn's disease, ulcerative colitis, and ulcers in the gastrointestinal tract or such as may occur in diseases of the gastrointestinal tract which are associated with increased secretions, such as Ménétrier's disease, secreting adenomas and protein loss syndrome, and also for treating nasal polyps and polyps of the gastrointestinal tract of various origins such as e.g., villous or adenomatous polyps of the large bowel, but also polyps in familial polyposis coli, intestinal polyps in Gardner's syndrome, polyps throughout the entire gastrointestinal tract in Peutz-Jeghers syndrome, in inflammatory pseudopolyps, juvenile polyps, Colitis cystica profunda, and Pneumatosis cystoides intestinales.

Moreover, the compounds of general formula I and the physiologically acceptable salts thereof may be used to treat kidney diseases, particularly in cystic changes such as cystic kidneys, for treating renal cysts which may be idiopathic in origin or occur in syndromes such as e.g., tuberculous sclerosis, in von-Hippel-Lindau Syndrome, in nephronophthisis and spongy kidney, and other diseases caused by aberrant function of tyrosine kinases, such as e.g., epidermal hyperproliferation (psoriasis), inflammatory processes, diseases of the immune system, hyperproliferation of hematopoietic cells, etc.

By reason of their biological properties the compounds according to the invention may be used on their own or in conjunction with other pharmacologically active compounds, for example in tumour therapy, in monotherapy or in conjunction with other anti-tumour therapeutic agents, for example in combination with topoisomerase inhibitors (e.g., etoposide), mitosis inhibitors (e.g., vinblastine), compounds which interact with nucleic acids (e.g., cisplatin, cyclophosphamide, adriamycin), hormone antagonists (e.g., tamoxifen), inhibitors of metabolic processes (e.g., 5-FU etc), cytokines (e.g., interferons), antibodies, etc. For treating respiratory tract diseases, these compounds may be used on their own or in conjunction with other therapeutic agents for the airways, such as substances with a secretolytic, broncholytic, and/or antiinflammatory activity. For treating diseases in the region of the gastrointestinal tract, these compounds may also be administered on their own or in conjunction with substances having an effect on motility or secretion or antiinflammatory substances. These combinations may be administered either simultaneously or sequentially.

These compounds may be administered either on their own or in conjunction with other active substances by intravenous, subcutaneous, intramuscular, intrarectal, intraperitoneal, or intranasal route, by inhalation, or transdermally or orally, wherein aerosol formulations are particularly suitable for inhalation.

For pharmaceutical use the compounds according to the invention are generally used for warm-blooded vertebrates, particularly humans, in doses of 0.01-100 mg/kg of body weight, preferably 0.1-15 mg/kg. For administration, they are formulated with one or more conventional inert carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearyl alcohol, carboxymethylcellulose, or fatty substances such as hard fat or suitable mixtures thereof in conventional galenic preparations such as plain or coated tablets, capsules, powders, suspensions, solutions, sprays, or suppositories.

The following Examples are intended to illustrate the present invention without restricting it.

Preparation of the Starting Compounds:

### EXAMPLE I

### 4-(3-chloro-4-fluorophenylamino)-6-[3-(4-tert-butyloxycarbonylpiperazino)propyloxyl]-7-methoxyquinazoline

500 mg of 4-(3-chloro-4-fluorophenylamino)-6-hydroxy-7-methoxyquinazoline, 600 mg of 1-[3-(methanesulfonyloxy)propyl]-4-tert-butyloxycarbonylpiperazine (prepared by reacting 1-(3-hydroxypropyl)-4-tert-butyloxycarbonylpiperazine with methanesulfonic acid anhydride in the presence of triethylamine) and 520 mg of potassium carbonate are stirred in 20 ml of dimethylformamide for 8 hours at 80° C. A further 300 mg of the piperazino compound is added and stirring is continued for another 4 hours at 80° C. The reaction mixture is concentrated by evaporation and the residue is divided between water and ethyl acetate. The organic phase is concentrated by evaporation and the residue is purified by chromatography on a silica gel column with ethyl acetate. Yield: 700 mg of (82% of theory); R_{f} value: 0.29 (silica gel; ethyl acetate/methanol=9:1); mass spectrum: (M−H)=544, 546

The following compounds are obtained analogously to Example I:

(1) 4-(3-chloro-4-fluorophenylamino)-6-[3-(1-tert-butyloxycarbonyl-4-piperidinyl)propyloxy]-7-methoxyquinazoline

R_{f} value: 0.70 (silica gel; ethyl acetate/methanol=9:1)

(2) (S)-4-[(3-bromophenyl)amino]-6-{[1-(tert-butyloxycarbonyl)pyrrolidine-2-yl]methoxy}-7-methoxyquinazoline

Melting point: 178° C.; mass spectrum (ESI⁻): m/z=527, 529 [M−H]⁻.

(3) (R)-4-[(3-bromophenyl)amino]-6-{[1-(tert-butyloxycarbonyl)pyrrolidine-2-yl]methoxy}-7-methoxyquinazoline

R_{f} value: 0.65 (silica gel, ethyl acetate/methanol=9:1); mass spectrum (ESI⁻): m/z=528, 530 [M]⁺.

(4) (S)-4-[(3-chloro-4-fluorophenyl)amino]-6-{[1-(tert-butyloxycarbonyl)pyrrolidin-2-yl]methoxy}-7-cyclopentyloxyquinazoline

R_{f} value: 0.76 (silica gel, methylene chloride/methanol/concentrated aqueous ammonia=90:10:0.1); mass spectrum (ESI⁻): m/z=555, 557 [M−H]⁻.

(5) (S)-4-[(3-chloro-4-fluorophenyl)amino]-6-{[1-(tert-butyloxycarbonyl)pyrrolidin-2-yl]methoxy}-7-cyclopentylmethoxyquinazoline

Melting point: 210° C.-211.5° C.; mass spectrum (ESI⁻): m/z=569, 571 [M−H]⁻.

### EXAMPLE II

### 4-(3-chloro-4-fluorophenylamino)-6-[3-(1-piperazinyl)propyloxyl-7-methoxyquinazoline

600 mg of 4-(3-chloro-4-fluorophenylamino)-6-[3-(4-tert-butyl-oxycarbonylpiperazino)propyloxy]-7-methoxyquinazoline in 5 ml methylene chloride is mixed with 1.5 ml of trifluoroacetic acid and stirred for 2 hours at ambient temperature. The reaction mixture is concentrated by evaporation and combined with 2N NaOH. It is decanted off the sticky residue, the residue is taken up in methanol, concentrated by evaporation and triturated with diethyl ether. Yield: 280 mg of (50% of theory); R_{f} value: 0.49 (aluminium oxide; ethyl acetate/methanol/concentrated aqueous ammonia=9:1:0.1); mass spectrum: (M+H)⁺.=446, 448.

The following compounds are obtained analogously to Example II:

(1) 4-(3-chloro-4-fluorophenylamino)-6-[3-(4-piperidinyl)propyloxy]-7-methoxyquinazoline

R_{f} value: 0.33 (aluminium oxide; ethyl acetate/methanol/concentrated aqueous ammonia=9:1:0.1); mass spectrum: (M+H)⁺.=445, 447

(2) (S)-4-[(3-bromophenyl)amino]-6-[(pyrrolidine-2-yl)methoxy]-7-methoxyquinazoline

Melting point: 143° C.; mass spectrum (ESI⁺): m/z=429, 431 [M+H]⁺.

(3) (R)-4-[(3-bromophenyl)amino]-6-[(pyrrolidine-2-yl)methoxy]-7-methoxyquinazoline

R_{f} value: 0.21 (silica gel, ethyl acetate/methanol/concentrated aqueous ammonia solution=9:1:0.1).

(4) (S)-4-[(3-chloro-4-fluorophenyl)amino]-6-[(pyrrolidin-2-yl)methoxy]-7-cyclopentyloxyquinazoline

R_{f} value: 0.18 (silica gel, methylene chloride/methanol/concentrated aqueous ammonia=90:10:0.1); mass spectrum (ESI⁻): m/z=455, 457 [M−H]⁻.

(5) (S)-4-[(3-chloro-4-fluorophenyl)amino]-6-[(pyrrolidin-2-yl)methoxy]-7-cyclopentylmethoxyquinazoline

R_{f} value: 0.36 (silica gel, methylene chloride/methanol/concentrated aqueous ammonia=90:10:0.1); mass spectrum (ESI⁺): m/z=471, 473 [M+H]⁺.

### EXAMPLE III

### N-(3-Brompropyl)sarcosine ethyl ester and N-(3-chloropropyl)sarcosine ethyl ester

6.9 ml of 1,3-dibromopropene in 20 ml acetonitrile is added dropwise to 2.4 g of sarcosine ethyl ester hydrochloride and 6 ml of N-ethyldiisopropylamine in 50 ml of acetonitrile. After stirring overnight at ambient temperature, the mixture is concentrated by evaporation and the residue is divided between ethyl acetate and water. The organic phase is concentrated by evaporation and the residue is purified by chromatography on silica gel (ethyl acetate/methanol=9:1). Yield: 0.77 g; R_{f} value: 0.80 (silica gel; ethyl acetate/methanol=9:1); mass spectrum: M⁺.=237, 239 and 193, 195.

The following compounds are obtained analogously to Example III:

(1) (S)—N-(3-Bromopropyl)proline methyl ester and (S)—N-(3-chloropropyl)proline methyl ester

R_{f} value: 0.84 (silica gel, ethyl acetate/methanol=9:1); mass spectrum (EI): m/z=249, 251 [M]^{+l and} 205, 207 [M]⁺.

(2) (R)—N-(3-bromopropyl)proline methyl ester and (R)—N-(3-chloropropyl)proline methyl ester

R_{f} value: 0.84 (silica gel, ethyl acetate/methanol=9:1); mass spectrum (EI): m/z=249, 251 [M]⁺ and 205, 207 [M]⁺.

### EXAMPLE IV

### 4-[(3-bromophenyl)amino]-6-(2-bromethoxy)-7-methoxyquinazoline

7.00 g of potassium carbonate and 8.70 ml of dibromoethane are added to 3.50 g of 4-[(3-bromophenyl)amino]-6-hydroxy-7-methoxyquinazoline in 350 ml dimethylformamide. The reaction mixture is stirred for two hours at 85° C. Then the mixture is concentrated by evaporation and the oily residue is stirred with methanol. The bright yellow precipitate formed is suction filtered and dried. Yield: 3.70 g (81% of theory); R_{f} value: 0.44 (silica gel, ethyl acetate); mass spectrum (ESI⁺): m/z=452, 454, 456 [M+H]⁺.

The following compounds are obtained analogously to Example IV:

(1) 4-[(3-chloro-4-fluorophenyl)amino]-6-(2-bromoethoxy)-7-cyclopentyloxyquinazoline

R_{f} value: 0.74 (silica gel, methylene chloride/methanol/concentrated aqueous ammonia=90:10:1); mass spectrum (ESI⁻): m/z=478, 480, 482 [M−H]⁻.

(2) 4-[(3-chloro-4-fluorophenyl)amino]-6-cyclopentyloxy-7-(2-bromoethoxy)quinazoline

R_{f} value: 0.65 (silica gel, methylene chloride/methanol/concentrated aqueous ammonia=90:10:0.1); mass spectrum (ESI⁻): m/z=478, 480, 482 [M−H]⁻.

### EXAMPLE V

### 4-[(3-bromophenyl)amino]-6-hydroxy-7-methoxyquinazoline

34.50 g of 4-[(3-bromophenyl)amino]-6-methylcarbonyloxy-7-methoxyquinazoline in 350 ml ethanol is mixed with 35 ml of 40% sodium hydroxide solution. The reaction mixture is stirred for three hours at ambient temperature. Then the mixture is concentrated by evaporation, the residue is taken up in water and neutralized with 2N hydrochloric acid. The precipitate formed is suction filtered and dried overnight in the circulating air drier at 50° C. Yield: 28.30 g (92% of theory); melting point: 299° C.; mass spectrum (ESI⁺): m/z=346, 348 [M+H]⁺.

The following compounds are obtained analogously to Example V:

(1) 4-[(3-chloro-4-fluorophenyl)amino]-6-benzyloxy-7-hydroxyquinazoline

The reaction is carried out with concentrated aqueous ammonia in methanol. R_{f} value: 0.54 (silica gel, methylene chloride/methanol=9:1); mass spectrum (ESI⁺): m/z=396, 398 [M+H]⁺.

(2) 4-[(3-chloro-4-fluorophenyl)amino]-6-cyclopentyloxy-7-hydroxyquinazoline

The reaction is carried out with concentrated aqueous ammonia in methanol. R_{f} value: 0.53 (silica gel, methylene chloride/methanol/concentrated aqueous ammonia=90:10:0.1); mass spectrum (ESI⁺): m/z=374, 376 [M+H]⁺.

### EXAMPLE VI

### 4-[(3-bromophenyl)amino]-6-methylcarbonyloxy-7-methoxyquinazoline

13.0 ml of 3-bromoaniline is added to 30.00 g of 4-chloro-6-methylcarbonyloxy-7-methoxyquinazoline in 600 ml isopropanol. The reaction mixture is refluxed for about four hours. The reaction mixture is then left to cool. The precipitate formed is suction filtered, washed thoroughly with cold isopropanol and dried. Yield: 34.57 g (75% of theory); melting point: 238° C.; mass spectrum (ESI⁺): m/z=388, 390 [M+H]⁺.

The following compounds are obtained analogously to Example VI:

(1) 4-[(3-chloro-4-fluorophenyl)amino]-6-benzyloxy-7-methylcarbonyloxyquinazoline

Melting point: 267° C.-268° C.; mass spectrum (ESI⁺): m/z=438, 440 [M+H]⁺.

(2) 4-[(3-chloro-4-fluorophenyl)amino]-6-cyclopentyloxy-7-methylcarbonyloxyquinazoline

R_{f} value: 0.73 (silica gel, methylene chloride/methanol/concentrated aqueous ammonia=90:10:0.1); mass spectrum (ESI⁺): m/z=416, 418 [M+H]⁺.

### EXAMPLE VII

### 4-[(3-bromophenyl)amino]-6-oxiranylmethoxy-7-methoxyquinazoline

1.50 ml of epibromohydrin is added to 5.00 g of 4-[(3-bromophenyl)amino]-6-hydroxy-7-methoxyquinazoline and 4.75 g of potassium carbonate in 50 ml dimethylsulfoxide. The reaction mixture is stirred for two days at 50° C. Then it is diluted with about 150 ml of water and stirred for a further two hours. The precipitate formed is suction filtered and purified by chromatography on a silica gel column with ethyl acetate as eluant. Yield: 850 mg (15% of theory); melting point: 230° C.-245° C.; mass spectrum (ESI⁺): m/z=402, 404 [M+H]⁺.

### EXAMPLE VIII

### Dimethyl 2-(piperazin-1-yl)succinate dihydrochloride

8.70 g of dimethyl 2-(4-benzylpiperazin-1-yl)succinate is hydrogenated in a mixture of 100 ml methanol and 4.50 ml of concentrated hydrochloric acid in the presence of 4.00 g of palladium (10% on activated charcoal) at ambient temperature until the calculated amount of hydrogen is taken up (about an hour). Then the catalyst is removed by suction filtering and the filtrate is concentrated by evaporation. A white gel-like solid is left. Yield: 4.18 g; R_{f} value: 0.80 (Reversed phase ready-made TLC plate (E. Merck), acetonitrile/water/trifluoroacetic acid=1:1:1); mass spectrum (ESI⁺): m/z=231 [M+H]⁺.

The following compound is obtained analogously to Example VIII:

(1) dimethyl 3-(piperazin-1-yl)-glutarate dihydrochloride

R_{f} value: 0.80 (Reversed phase ready-made TLC plate (E. Merck), acetonitrile/water/trifluoroacetic acid=1:1:1); mass spectrum (ESI⁺): m/z=254 [M+H]⁺.

### EXAMPLE IX

### Dimethyl 2-(4-benzylpiperazin-1-yl)succinate

7.22 ml of dimethyl maleate is added to 10.0 ml of N-benzylpiperazine in 15 ml dioxane. The reaction mixture is stirred for half an hour at ambient temperature. Then the mixture is refluxed for about a further three hours. For working up the reaction mixture is evaporated to dryness. An orange-yellow oil remains, which slowly crystallizes. Yield: 21.3 g (crude product); R_{f} value: 0.85 (silica gel, methylene chloride/methanol/concentrated aqueous ammonia solution=90:10:0.5); mass spectrum (EI): m/z=320 [M]⁺.

The following compound is obtained analogously to Example IX:

(1) dimethyl 3-(4-benzylpiperazin-1-yl)-glutarate (reaction with dimethyl glutaconate)

R_{f} value: 0.49 (silica gel, cyclohexane/ethyl acetate=1:1); mass spectrum (EI): m/z=334 [M]⁺.

### EXAMPLE X

### 4-[(3-Chloro-4-fluorophenyl)amino]-6-hydroxy-7-cyclopentyloxyquinazoline

10 ml of trifluoroacetic acid is added to 1.95 g of 4-[(3-chloro-4-fluorophenyl)amino]-6-benzyloxy-7-cyclopentyloxyquinazoline and the resulting dark brown solution is stirred at room temperature over night. Another 5 ml of trifluoroacetic acid is added and the mixture is stirred for approximately 2.5 hours at 50° C. until the reaction is completed. The reaction mixture is concentrated in vacuo, diluted with water, and adjusted to pH 8-9 by addition of concentrated aqueous ammonia. The precipitate is filtered off with suction, washed with water, and dried in vacuo at 60° C. Yield: 1.45 g (92% of theory); R_{f} value: 0.56 (silica gel, methylene chloride/methanol 9:1); mass spectrum (ESI⁻): m/z=372, 374 [M−H]⁻.

The following compound is obtained analogously to Example X:

(1) 4-[(3-chloro-4-fluorophenyl)amino]-6-hydroxy-7-cyclopentylmethoxyquinazoline

R_{f} value: 0.73 (silica gel, methylene chloride/methanol/concentrated aqueous ammonia=90:10:0.1); mass spectrum (ESI⁻): m/z=386, 388 [M−H]⁻.

### EXAMPLE XI

### 4-[(3-chloro-4-fluorophenyl)amino]-6-benzyloxy-7-cyclopentyloxyquinazoline

0.65 ml of bromocyclopentane is added to a mixture of 2.30 g 4-[(3-chloro-4-fluorophenyl)amino]-6-benzyloxy-7-hydroxyquinazoline and 6.00 g potassium carbonate in 6 ml of N,N-dimethylformamide and the reaction mixture is stirred for 18 hours at room temperature. Another 3.00 g of potassium carbonate and 4 drops of bromocyclopentane are added, and the resulting mixture is stirred for 2.5 hours at 50° C. The reaction mixture is partitioned between ethyl acetate and water, and the aqueous layer is extracted with ethyl acetate. The combined organic extracts are washed with concentrated aqueous sodium chloride solution, dried over magnesium sulfate and concentrated in vacuo. The oily residue is triturated with methanol, the resulting solid precipitate is filtered off, washed with cold methanol, and dried in vacuo. Yield: 2.09 g (77% of theory); R_{f} value: 0.63 (silica gel, methylene chloride/methanol 9:1); mass spectrum (ESI⁻): m/z=462, 464 [M−H]⁻.

The following compound is obtained analogously to Example XI:

(1) 4-[(3-chloro-4-fluorophenyl)amino]-6-benzyloxy-7-cyclopentylmethoxyquinazoline

R_{f} value: 0.84 (silica gel, methylene chloride/methanol/concentrated aqueous ammonia=90:10:1); mass spectrum (ESI⁺): m/z=478, 480 [M+H]⁺.

### EXAMPLE XII

### 4-chloro-6-benzyloxy-7-methylcarbonyloxyquinazoline

Prepared by reaction of 6-benzyloxy-7-methylcarbonyloxy-3H-quinazolin-4-one with thionyl chloride in the presence of catalytic amounts of N,N-dimethylformamide. Yield: 98% of theory; R_{f} value: 0.86 (silica gel, methylene chloride/methanol/concentrated aqueous ammonia=90:10:0.1)

The following compound is obtained analogously to Example XII:

(1) 4-chloro-6-cyclopentyloxy-7-methylcarbonyloxyquinazoline

R_{f} value: 0.69 (silica gel, methylene chloride/methanol/concentrated aqueous ammonia=90:10:0.1)

### EXAMPLE XIII

### 6-benzyloxy-7-methylcarbonyloxy-3H-quinazolin-4-one

Prepared by reaction of 6-benzyloxy-7-hydroxy-3H-quinazolin-4-one with acetic anhydride in pyridine. Yield: 68% of theory; melting point: 231° C.-233° C.; mass spectrum (ESI⁻): m/z=309 [M−H]⁻.

The following compound is obtained analogously to Example XIII:

(1) 6-cyclopentyloxy-7-methylcarbonyloxy-3H-quinazolin-4-one

R_{f} value: 0.57 (silica gel, methylene chloride/methanol/concentrated aqueous ammonia=90:10:0.1); mass spectrum (ESI): m/z=287 [M−H]⁻.

### EXAMPLE XIV

### 6-Benzyloxy-7-hydroxy-3H-quinazolin-4-one

Prepared by reaction of 2-amino-4-hydroxy-5-benzyloxybenzoic acid with formamidine acetate in ethanol. Yield: 72% of theory; R_{f} value: 0.45 (silica gel, methylene chloride/methanol/concentrated aqueous ammonia=90:10:0.1); mass spectrum (ESI⁻): m/z=267 [M−H]⁻.

The following compound is obtained analogously to Example XIV:

(1) 6-cyclopentyloxy-7-hydroxy-3H-quinazolin-4-one

R_{f} value: 0.42 (silica gel, methylene chloride/methanol/concentrated aqueous ammonia=90:10:0.1); mass spectrum (EI): m/z=246 [M]⁺.

### EXAMPLE XV

### 2-Amino-4-hydroxy-5-benzyloxybenzoic acid

Prepared by catalytic hydrogenation of 2-nitro-4-hydroxy-5-benzyloxybenzoic acid with Raney nickel in methanol. Yield: 71% of theory; R_{f} value: 0.53 (silica gel, methylene chloride/methanol/concentrated aqueous ammonia=90:10:0.1); mass spectrum (ESI⁻): m/z=258 [M−H]⁻.

The following compound is obtained analogously to Example XV:

(1) 2-amino-4-hydroxy-5-cyclopentyloxybenzoic acid

R_{f} value: 0.38 (silica gel, methylene chloride/methanol/concentrated aqueous ammonia=90:10:0.1); mass spectrum (ESI⁻): m/z=236 [M−H]⁻.

### EXAMPLE XVI

### 2-Nitro-4-hydroxy-5-benzyloxybenzoic acid

4.8 g of sodium is added portionwise to a mixture of 20.30 g 6-nitro-benzo[1,3]dioxole-5-carboxylic acid and 81.2 ml of benzyl alcohol in 120 ml of dimethyl sulfoxide cooled in an ice/water bath. The reaction mixture is allowed to warm up to room temperature and stirred for approximately 21 hours. The brownish red solution is diluted with 600 ml of water and extracted with methylene chloride. The aqueous layer is acidified with concentrated hydrochloric acid and stirred for two hours at room temperature. The precipitate is filtered off, washed with water, and dried. Yield: 18.63 g (67% of theory); melting point: 172° C.-175° C.; mass spectrum (ESI⁻): m/z=288 [M−H]⁻.

The following compound is obtained analogously to Example XVI:

(1) 2-nitro-4-hydroxy-5-cyclopentyloxybenzoic acid

R_{f} value: 0.61 (silica gel, toluene/1,4-dioxane/ethanol/acetic acid=90:10:10:6); mass spectrum (ESI⁻): m/z=266 [M−H]⁻.

Preparation of the End Products:

### EXAMPLE 1

### 4-(3-chloro-4-fluorophenylamino)-6-{3-[4-(methoxycarbonylmethyl)-1-piperazinyl]propyloxy}-7-methoxyquinazoline

0.07 ml of methyl bromoacetate in 1 ml of acetonitrile is added dropwise to 250 mg of 4-(3-chloro-4-fluorophenylamino)-6-[3-(1-piperazinyl)propyloxy]-7-methoxyquinazoline and 0.13 ml N-ethyldiisopropylamine in 5 ml of acetonitrile. After 2 hours' stirring at ambient temperature, the mixture is concentrated by evaporation, mixed with water and extracted with ethyl acetate. The organic phases are washed with saline solution, then dried with magnesium sulfate and concentrated by evaporation. Yield: 150 mg (51% of theory); R_{f} value: 0.54 (silica gel; ethyl acetate/methanol/concentrated aqueous ammonia=9:1:0.1); mass spectrum: (M−H)=516, 518.

The following compounds are obtained analogously to Example 1:

(1) 4-(3-chloro-4-fluorophenylamino)-6-{3-[1-(methoxycarbonylmethyl)-4-piperidinyl]propyloxy}-7-methoxyquinazoline

R_{f} value: 0.79 (silica gel; ethyl acetate/methanol/concentrated aqueous ammonia=9:1:0.1); mass spectrum: M⁺.=516, 518

(2) (S)-4-[(3-bromophenyl)amino]-6-({1-[(ethoxycarbonyl)methyl]pyrrolidin-2-yl}methoxy)-7-methoxyquinazoline

R_{f} value: 0.68 (silica gel, ethyl acetate/methanol/concentrated aqueous ammonia solution=9:1:0.1); mass spectrum (EI): m/z=514, 516 [M]⁺.

(3) (R)-4-[(3-bromophenyl)amino]-6-({1-[(ethoxycarbonyl)methyl]pyrrolidin-2-yl}methoxy)-7-methoxyquinazoline

R_{f} value: 0.75 (silica gel, ethyl acetate/methanol=9:1); mass spectrum (EI): m/z=514, 516 [M]⁺.

(4) (S)-4-[(3-chloro-4-fluorophenyl)amino]-6-({1-(methoxycarbonyl)methyl]pyrrolidin-2-yl}methoxy)-7-cyclopentyloxyquinazoline

R_{f} value: 0.59 (silica gel, methylene chloride/methanol/concentrated aqueous ammonia=90:10:0.1); mass spectrum (ESI⁻): m/z=527, 529 [M−H]⁻.

(5) (S)-4-[(3-chloro-4-fluorophenyl)amino]-6-({1-(methoxycarbonyl)methyl]pyrrolidin-2-yl}methoxy)-7-cyclopentylmethoxyquinazoline

R_{f} value: 0.67 (silica gel, methylene chloride/methanol/concentrated aqueous ammonia=90:10:0.1); mass spectrum (ESI⁻): m/z=541, 543 [M−H]⁻.

### EXAMPLE 2

### 4-(3-chloro-4-fluorophenylamino)-6-{3-[N-(ethoxycarbonylmethyl)-N-methylamino]propyloxy}-7-methoxyquinazoline

380 mg of a mixture of N-(3-bromopropyl)sarcosine ethyl ester and N-(3-chloropropyl)sarcosine ethyl ester in 5 ml dimethylformamide is added dropwise to 500 mg of 4-(3-chloro-4-fluorophenylamino)-6-hydroxy-7-methoxyquinazoline and 220 mg of potassium tert-butoxide in 15 ml dimethylformamide. After 3 hours' stirring at 80° C. and standing overnight, a further 110 mg of potassium tert-butoxide and 190 mg of the sarcosine mixture are added and the reaction mixture is stirred for 4 hours at 80° C. It is filtered, the filtrate is concentrated by evaporation, the residue is taken up in water and extracted with ethyl acetate. The organic phase is separated off, dried and concentrated by evaporation. The residue is purified by chromatography on a silica gel column. Yield: 390 mg of (52% of theory); R_{f} value: 0.68 (silica gel; ethyl acetate/methanol/concentrated aqueous ammonia=9:1:0.1); mass spectrum: (M−H)=475, 477

The following compounds are obtained analogously to Example 2:

(1) (S)-4-[(3-bromophenyl)amino]-6-[3-(2-methoxycarbonylpyrrolidin-1-yl)propyloxy]-7-methoxyquinazoline

R_{f} value: 0.38 (silica gel, ethyl acetate/methanol=9:1); mass spectrum (EI): m/z=514, 516 [M]⁺.

(2) (R)-4-[(3-bromophenyl)amino]-6-[3-(2-methoxycarbonyl-pyrrolidin-1-yl)propyloxy]-7-methoxyquinazoline

R_{f} value: 0.41 (silica gel, ethyl acetate/methanol=9:1); mass spectrum (EI): m/z=514, 516 [M]⁺.

### EXAMPLE 3

### 4-[(3-bromophenyl)amino]-6-(2-{4-[(ethoxycarbonyl)methyl]piperazin-1-yl}ethoxy)-7-methoxyquinazoline

1.50 ml of diisopropyl-ethylamine and 1.10 ml of 1-[(ethoxycarbonyl)methyl]piperazine is added to 1.00 g of 4-[(3-bromophenyl)amino]-6-(2-bromoethoxy)-7-methoxyquinazoline in 20 ml acetonitrile. The reaction mixture is stirred for two days at ambient temperature. The precipitate formed is filtered off and the filtrate is concentrated by evaporation. The residue is taken up in ethyl acetate and washed once with saturated sodium hydrogen carbonate solution and once with water. The organic phase is dried over magnesium sulfate and concentrated by evaporation. The crude product is purified on a silica gel column with ethyl acetate/ethanol/concentrated aqueous ammonia solution (9:1:0.1) as eluant. Yield: 450 mg of (38% of theory); melting point: 155° C.; mass spectrum (EI): m/z=543, 545 [M]⁺.

The following compounds are obtained analogously to Example 3:

(1) 4-[(3-bromophenyl)amino]-6-(2-{N-[(ethoxycarbonyl)methyl]-N-methylamino}ethoxy)-7-methoxyquinazoline

R_{f} value: 0.55 (silica gel, ethyl acetate/methanol=9:1); mass spectrum (EI): m/z=488, 490 [M]⁺.

(2) 4-[(3-bromophenyl)amino]-6-(2-{N,N-bis[(ethoxycarbonyl)methyl]amino}ethoxy)-7-methoxyquinazoline

R_{f} value: 0.38 (silica gel, ethyl acetate); mass spectrum (EI): m/z=560, 562 [M]⁺.

(3) 4-[(3-bromophenyl)amino]-6-(2-{4-[1,2-bis(methoxycarbonyl)ethyl]piperazin-1-yl}ethoxy)-7-methoxyquinazoline

R_{f} value: 0.61 (silica gel, ethyl acetate/methanol=9:1); mass spectrum (EI): m/z=601, 603 [M]⁺.

(4) 4-[(3-bromophenyl)amino]-6-[2-(4-{1-[(methoxycarbonyl)methyl]-2-(methoxycarbonyl)ethyl}piperazin-1-yl)ethoxy]-7-methoxyquinazoline

R_{f} value: 0.51 (silica gel, ethyl acetate/methanol=9:1); mass spectrum (ESI⁺): m/z=616, 618 [M+H]⁺.

(5) (R)-4-[(3-chloro-4-fluorophenyl)amino]-6-{2-[2-(methoxycarbonyl)pyrrolidin-1-yl]ethoxy}-7-cyclopentyloxyquinazoline

R_{f} value: 0.65 (silica gel, methylene chloride/methanol/concentrated aqueous ammonia=90:10:0.1); mass spectrum (ESI⁻): m/z=527, 529 [M−H]⁻.

(6) 4-[(3-chloro-4-fluorophenyl)amino]-6-(2-{4-[(ethoxycarbonyl)methyl]piperazin-1-yl-}-ethoxy)-7-cyclopentyloxyquinazoline

R_{f} value: 0.54 (silica gel, methylene chloride/methanol/concentrated aqueous ammonia=90:10:0.1); mass spectrum (ESI⁻): m/z=570, 572 [M−H]⁻.

(7) 4-[(3-chloro-4-fluorophenyl)amino]-6-cyclopentyloxy-7-(2-{N-(2-hydroxy-2-methylprop-1-yl)-N-[(ethoxycarbonyl)methyl]amino}ethoxy)quinazoline

R_{f} value: 0.28 (silica gel, ethyl acetate); mass spectrum (ESI⁻): m/z=573, 575 [M−H]⁻.

(8) 4-[(3-chloro-4-fluorophenyl)amino]-6-cyclopentyloxy-7-[2-(6,6-dimethyl-2-oxomorpholin-4-yl)ethoxy]quinazoline

This compound was obtained by treatment of the compound prepared by example 3(7) with toluene-4-sulfonic acid in toluene. R_{f} value: 0.23 (silica gel, ethyl acetate); mass spectrum (ESI⁻): m/z=527, 529 [M−H]⁻.

(9) 4-[(3-chloro-4-fluorophenyl)amino]-6-cyclopentyloxy-7-{2-[N-(2-oxotetrahydrofuran-3-yl)-N-methylamino]ethoxy}quinazoline

The starting material 3-methylaminodihydrofuran-2-one was prepared by reaction of 3-bromodihydrofuran-2-one with N-methylbenzylamine and subsequent hydrogenolytic removal of the benzyl group). R_{f} value: 0.42 (silica gel, ethyl acetate/methanol=9:1); mass spectrum (ESI⁺): m/z=515, 517 [M+H]⁺.

(10) 4-[(3-bromophenyl)amino]-6-(2-{N-(2-hydroxy-2-methylprop-1-yl)-N-[(ethoxycarbonyl)methyl]amino}ethoxy)-7-methoxyquinazoline

(11) 4-[(3-bromophenyl)amino]-6-[2-(6,6-dimethyl-2-oxomorpholin-4-yl)ethoxy]-7-methoxyquinazoline

R_{f} value: 0.33 (silica gel, ethyl acetate); mass spectrum (ESI⁻): m/z=499, 500 [M+H]⁻.

(12) 4-[(3-bromophenyl)amino]-6-{2-[N-(2-oxotetrahydrofuran-4-yl)-N-methylamino]ethoxy}-7-methoxyquinazoline

The starting material 4-methylaminodihydrofuran-2-one was prepared by reaction of 5H-furan-2-one with N-methylbenzylamine and subsequent hydrogenolytic removal of the benzyl group. R_{f} value: 0.38 (silica gel, ethyl acetate/methanol=9:1); mass spectrum (ESI⁻): m/z=485, 487 [M−H]⁻.

### EXAMPLE 4

### 4-[(3-bromophenyl)amino]-6-(3-{4-[(ethoxycarbonyl)methyl]piperazin-1-yl}-2-hydroxypropyloxy)-7-methoxyquinazoline

0.16 ml of 1-[(ethoxycarbonyl)methyl]piperazine is added to 500 mg of 4-[(3-bromophenyl)amino]-6-oxiranylmethoxy-7-methoxyquinazoline in 5 ml ethanol. The reaction mixture is refluxed for about 6 hours. Then the mixture is concentrated by evaporation and the crude product is purified by chromatography on a silica gel column with ethyl acetate/ethanol/concentrated aqueous ammonia solution (9:1:0.1) as eluant. Yield: 97 mg of (14% of theory); melting point: 118° C.-122° C.; mass spectrum (EI): m/z=573, 575 [M]⁺.

### EXAMPLE 5

### 4-[(3-bromophenyl)amino]-6-{2-[4-(carboxymethyl)piperazin-1-yl]ethoxy}-7-methoxyquinazoline

0.19 ml of 1N sodium hydroxide solution is added to 100 mg of 4-[(3-bromophenyl)amino]-6-(2-{4-[(ethoxycarbonyl)methyl]piperazin-1-yl}ethoxy)-7-methoxyquinazoline in 0.30 ml of tetrahydrofuran. The reaction mixture is stirred for three hours at ambient temperature. Another 0.9 ml of 1N sodium hydroxide solution is added and the mixture is stirred overnight. Then it is neutralized with 1N hydrochloric acid and concentrated by evaporation. The solid residue is triturated with ethyl acetate and suction filtered. Yield: 100 mg (contains about 0.5 equivalents sodium chloride); R_{f} value: 0.50 (Reversed phase ready-made TLC plate (E. Merck), acetonitrile/water/trifluoroacetic acid=50:50:1); mass spectrum (ESI⁻): m/z=514, 516 [M−H]⁻.

The following compounds may also be obtained analogously to the foregoing Examples and other methods known from the literature:
(1) 4-[(3-bromophenyl)amino]-6-({1-[(methoxycarbonyl)methyl]piperidin-4-yl}methoxy)-7-methoxyquinazoline;
(2) 4-[(3-methylphenyl)amino]-6-({1-[(ethoxycarbonyl)methyl]piperidin-4-yl}methoxy)-7-methoxyquinazoline;
(3) 4-[(3-chlorophenyl)amino]-6-({1-[(ethoxycarbonyl)methyl]piperidin-4-yl}methoxy)-7-methoxyquinazoline;
(4) 4-[(3-chloro-4-fluorophenyl)amino]-6-({1-[(ethoxycarbonyl)methyl]piperidin-4-yl}methoxy)-7-methoxyquinazoline;
(5) 4-[(indol-5-yl)amino]-6-({1-[(ethoxycarbonyl)methyl]piperidin-4-yl}methoxy)-7-methoxyquinazoline;
(6) 4-[(1-phenylethyl)amino]-6-({1-[(ethoxycarbonyl)methyl]piperidin-4-yl}methoxy)-7-methoxyquinazoline;
(7) 4-[(3-ethynylphenyl)amino]-6-({1-[(ethoxycarbonyl)methyl]piperidin-4-yl}methoxy)-7-methoxyquinazoline;
(8) 4-[(3-bromophenyl)amino]-6-({1-[(ethoxycarbonyl)methyl]piperidin-4-yl}methoxy)-7-methoxyquinazoline;
(9) 4-[(3-bromophenyl)amino]-6-({1-[(hexyloxycarbonyl)methyl]piperidin-4-yl}methoxy)-7-methoxyquinazoline;
(10) 4-[(3-bromophenyl)amino]-6-({1-[2-(ethoxycarbonyl)ethyl]piperidin-4-yl}methoxy)-7-methoxyquinazoline;
(11) 4-[(3-bromophenyl)amino]-6-({1-[3-(ethoxycarbonyl)propyl]piperidin-4-yl}methoxy)-7-methoxyquinazoline;
(12) 4-[(3-bromophenyl)amino]-6-({1-[(ethoxycarbonyl)methyl]piperidin-3-yl}methoxy)-7-methoxyquinazoline;
(13) 4-[(3-bromophenyl)amino]-6-({1-[(ethoxycarbonyl)methyl]pyrrolidin-2-yl}methoxy)-7-methoxyquinazoline;
(14) 4-[(3-bromophenyl)amino]-6-({1-[(dimethoxyphosphoryl)methyl]piperidin-4-yl}methoxy)-7-methoxyquinazoline;
(15) 4-[(3-bromophenyl)amino]-6-[(1-{[(methoxy)(methyl)phosphoryl]methyl}piperidin-4-yl)methoxy]-7-methoxyquinazoline;
(16) 4-[(3-bromophenyl)amino]-6-({1-[1,2-bis(ethoxycarbonyl)ethyl]piperidin-4-yl}methoxy)-7-methoxyquinazoline;
(17) 4-[(3-bromophenyl)amino]-6-[(1-{1-[(ethoxycarbonyl)methyl]-2-(ethoxycarbonyl)ethyl}piperidin-4-yl)methoxy]-7-methoxyquinazoline;
(18) 4-[(3-bromophenyl)amino]-6-(2-{1-[1-(methoxycarbonyl)ethyl]piperidin-4-yl}ethoxy)-7-methoxyquinazoline;
(19) 4-[(3-bromophenyl)amino]-6-(2-{1-[(methoxycarbonyl)methyl]piperidin-4-yl}ethoxy)-7-methoxyquinazoline;
(20) 4-[(3-bromophenyl)amino]-6-(2-{4-[(methoxycarbonyl)methyl]piperazin-1-yl}ethoxy)-7-methoxyquinazoline;
(21) 4-[(3-bromophenyl)amino]-6-(2-{4-[(ethoxycarbonyl)methyl]piperazin-1-yl}ethoxy)-7-methoxyquinazoline;
(22) 4-[(3-bromophenyl)amino]-6-(2-{1-[(ethoxycarbonyl)methyl]piperidin-4-yl}ethoxy)-7-methoxyquinazoline;
(23) 4-[(3-bromophenyl)amino]-6-(2-{1-[1,2-bis(ethoxycarbonyl)ethyl]piperidin-4-yl}ethoxy)-7-methoxyquinazoline;
(24) 4-[(3-bromophenyl)amino]-6-(2-{4-[1,2-bis(ethoxycarbonyl)ethyl]piperazin-1-yl}ethoxy)-7-methoxyquinazoline;
(25) 4-[(3-bromophenyl)amino]-6-[2-(4-{1-[(ethoxycarbonyl)methyl]-2-(ethoxycarbonyl)ethyl}piperazin-1-yl)ethoxy]-7-methoxyquinazoline;
(26) 4-[(3-bromophenyl)amino]-6-[2-(1-{1-[(ethoxycarbonyl)methyl]-2-(ethoxycarbonyl)ethyl}piperidin-4-yl)ethoxy]-7-methoxyquinazoline;
(27) 4-[(3-bromophenyl)amino]-6-{2-[2-(methoxycarbonyl)pyrrolidin-1-yl]ethoxy}-7-methoxyquinazoline;
(28) 4-[(3-bromophenyl)amino]-6-{2-[2-(ethoxycarbonyl)piperidin-1-yl]ethoxy}-7-methoxyquinazoline;
(29) 4-[(3-bromophenyl)amino]-6-(3-{1-[(methoxycarbonyl)methyl]piperidin-4-yl}propyloxy)-7-methoxyquinazoline;
(30) 4-[(3-bromophenyl)amino]-6-(3-{4-[(methoxycarbonyl)methyl]piperazin-1-yl}propyloxy)-7-methoxyquinazoline;
(31) 4-[(3-bromophenyl)amino]-6-(3-{4-[(ethoxycarbonyl)methyl]piperazin-1-yl}propyloxy)-7-methoxyquinazoline;
(32) 4-[(3-bromophenyl)amino]-6-(3-{1-[(ethoxycarbonyl)methyl]piperidin-4-yl}propyloxy)-7-methoxyquinazoline;
(33) 4-[(3-bromophenyl)amino]-6-(3-{1-[(ethoxycarbonyl)methyl]piperidin-4-yl}-2-hydroxypropyloxy)-7-methoxyquinazoline;
(34) 4-[(3-bromophenyl)amino]-6-(3-{4-[(ethoxycarbonyl)methyl]piperazin-1-yl}-2-hydroxypropyloxy)-7-methoxyquinazoline;
(35) 4-[(3-methylphenyl)amino]-6-(3-{4-[(ethoxycarbonyl)methyl]piperazin-1-yl}propyloxy)-7-methoxyquinazoline;
(36) 4-[(3-chlorophenyl)amino]-6-(3-{4-[(ethoxycarbonyl)methyl]piperazin-1-yl}propyloxy)-7-methoxyquinazoline;
(37) 4-[(indol-5-yl)amino]-6-(3-{4-[(ethoxycarbonyl)methyl]piperazin-1-yl}propyloxy)-7-methoxyquinazoline;
(38) 4-[(1-phenylethyl)amino]-6-(3-{4-[(ethoxycarbonyl)methyl]piperazin-1-yl}propyloxy)-7-methoxyquinazoline;
(39) 4-[(3-bromophenyl)amino]-6-{3-[2-(methoxycarbonyl)pyrrolidin-1-yl]propyloxy}-7-methoxyquinazoline;
(40) 4-[(3-bromophenyl)amino]-6-{3-[3-(methoxycarbonyl)-4-methyl-piperazin-1-yl]propyloxy}-7-methoxyquinazoline;
(41) 4-[(3-bromophenyl)amino]-6-({1-[(ethoxycarbonyl)methyl]piperidin-4-yl}methoxy)-7-ethoxyquinazoline;
(42) 4-[(3-bromophenyl)amino]-6-({1-[(ethoxycarbonyl)methyl]piperidin-4-yl}methoxy)-7-(2-methoxyethoxy)quinazoline;
(43) 4-[(3-bromophenyl)amino]-6-(2-{1-[(ethoxycarbonyl)methyl]piperidin-4-yl}ethoxy)-7-(2-methoxyethoxy)quinazoline;
(44) 4-[(3-bromophenyl)amino]-6-(2-{4-[(ethoxycarbonyl)methyl]piperazin-1-yl}ethoxy)-7-(2-methoxyethoxy)quinazoline;
(45) 4-[(3-bromophenyl)amino]-6-(2-{4-[(ethoxycarbonyl)methyl]piperazin-1-yl}ethoxy)-7-ethoxyquinazoline;
(46) 4-[(3-bromophenyl)amino]-6-(3-{1-[(ethoxycarbonyl)methyl]piperidin-4-yl}propyloxy)-7-ethoxyquinazoline;
(47) 4-[(3-bromophenyl)amino]-6-(3-{4-[(ethoxycarbonyl)methyl]piperazin-1-yl}propyloxy)-7-(2-methoxyethoxy)quinazoline;
(48) 4-[(3-bromophenyl)amino]-6-(3-{1-[(dimethoxyphosphoryl)methyl]piperidin-4-yl}propyloxy)-7-methoxyquinazoline;
(49) 4-[(3-bromophenyl)amino]-6-(3-{4-[(dimethoxyphosphoryl)methyl]piperazin-1-yl}propyloxy)-7-methoxyquinazoline;
(50) 4-[(3-bromophenyl)amino]-6-[3-(4-{[(methoxy)(ethyl)phosphoryl]methyl}piperazin-1-yl)propyloxy]-7-methoxyquinazoline;
(51) 4-[(3-bromophenyl)amino]-6-[3-(1-{[(methoxy)(ethyl)phosphoryl]methyl}piperidin-4-yl)propyloxy]-7-methoxyquinazoline;
(52) 4-[(3-bromophenyl)amino]-6-(3-{4-[1,2-bis(ethoxycarbonyl)ethyl]piperazin-1-yl}propyloxy)-7-methoxyquinazoline;
(53) 4-[(3-bromophenyl)amino]-6-[3-(1-{1-[(ethoxycarbonyl)methyl]-2-(ethoxycarbonyl)ethyl}piperidin-4-yl)propyloxy]-7-methoxyquinazoline;
(54) 4-[(3-bromophenyl)amino]-6-(4-{1-[(ethoxycarbonyl)methyl]piperidin-4-yl}butyloxy)-7-methoxyquinazoline;
(55) 4-[(3-bromophenyl)amino]-6-(4-{4-[(ethoxycarbonyl)methyl]piperazin-1-yl}butyloxy)-7-methoxyquinazoline;
(56) 4-[(3-bromophenyl)amino]-6-(2-{N-[(ethoxycarbonyl)methyl]-N-methylamino}ethoxy)-7-methoxyquinazoline;
(57) 4-[(3-bromophenyl)amino]-6-(2-{N,N-bis[(ethoxycarbonyl)methyl]amino}ethoxy)-7-methoxyquinazoline;
(58) 4-[(3-bromophenyl)amino]-6-(2-{N-[(ethoxycarbonyl)methyl]-N-ethylamino}ethoxy)-7-methoxyquinazoline;
(59) 4-[(3-bromophenyl)amino]-6-(2-{N-[(ethoxycarbonyl)methyl]-N-(cyclopropylmethyl)amino}ethoxy)-7-methoxyquinazoline;
(60) 4-[(3-bromophenyl)amino]-6-(2-{[(ethoxycarbonyl)methyl]amino}ethoxy)-7-methoxyquinazoline;
(61) 4-[(3-bromophenyl)amino)-6-(2-{N-[(ethoxycarbonyl)methyl]-N-cyclopropylamino}ethoxy)-7-methoxyquinazoline;
(62) 4-[(3-bromophenyl)amino]-6-(2-{N-[(methoxycarbonyl)methyl]-N-methylamino}ethoxy)-7-methoxyquinazoline;
(63) 4-[(3-bromophenyl)amino]-6-(3-{N-[(methoxycarbonyl)methyl]-N-methylamino}propyloxy)-7-methoxyquinazoline;
(64) 4-[(3-bromophenyl)amino]-6-(3-{N,N-bis[(methoxycarbonyl)methyl]amino}propyloxy)-7-methoxyquinazoline;
(65) 4-[(3-bromophenyl)amino]-6-(3-{[(ethoxycarbonyl)methyl]amino}propyloxy)-7-methoxyquinazoline;
(66) 4-[(3-bromophenyl)amino]-6-(4-{N-[(ethoxycarbonyl)methyl]-N-methylamino}butyloxy)-7-methoxyquinazoline;
(67) 4-[(3-bromophenyl)amino]-6-(4-{N,N-bis[(ethoxycarbonyl)methyl]amino}butyloxy)-7-methoxyquinazoline;
(68) 4-[(3-bromophenyl)amino]-6-({4-[(methoxycarbonyl)methyl]-2-oxomorpholin-6-yl}methyloxy)-7-methoxyquinazoline;
(69) 4-[(3-bromophenyl)amino]-6-[(4-methyl-2-oxomorpholin-6-yl)methyloxy]-7-methoxyquinazoline;
(70) 4-[(3-bromophenyl)amino]-6-[(2-oxomorpholin-6-yl)methyloxy]-7-methoxyquinazoline;
(71) 4-[(4-amino-3,5-dibromophenyl)amino]-6-(3-{4-[(methoxycarbonyl)methyl]piperazin-1-yl}propyloxy)-7-methoxyquinazoline;
(72) 4-[(4-amino-3,5-dibromophenyl)amino]-6-(3-{1-[(methoxycarbonyl)methyl]piperidin-4-yl}propyloxy)-7-methoxyquinazoline;
(73) 4-[(3-bromophenyl)amino]-6,7-bis(2-{N-[(ethoxycarbonyl)methyl]-N-methylamino}ethoxy)quinazoline;
(74) 4-[(3-bromophenyl)amino]-6,7-bis(3-{N-[(ethoxycarbonyl)methyl]-N-methylamino}propyloxy)quinazoline;
(75) 4-[(3-bromophenyl)amino]-6-[3-(morpholino)propyloxy]-7-[(ethoxycarbonyl)methoxy]quinazoline;
(76) 4-[(3-bromophenyl)amino]-6-[2-(morpholino)ethoxy]-7-[(ethoxycarbonyl)methoxy]quinazoline;
(77) 4-[(3-bromophenyl)amino]-7-({1-[(methoxycarbonyl)methyl]piperidin-4-yl}methoxy)-6-methoxyquinazoline;
(78) 4-[(3-methylphenyl)amino]-7-({1-[(ethoxycarbonyl)methyl]piperidin-4-yl}methoxy)-6-methoxyquinazoline;
(79) 4-[(3-chlorophenyl)amino]-7-({1-[(ethoxycarbonyl)methyl]piperidin-4-yl}methoxy)-6-methoxyquinazoline;
(80) 4-[(3-chloro-4-fluorophenyl)amino]-7-({1-[(ethoxycarbonyl)methyl]piperidin-4-yl}methoxy)-6-methoxyquinazoline;
(81) 4-[(indol-5-yl)amino]-7-({1-[(ethoxycarbonyl)methyl]piperidin-4-yl}methoxy)-6-methoxyquinazoline;
(82) 4-[(1-phenylethyl)amino]-7-({1-[(ethoxycarbonyl)methyl]piperidin-4-yl}methoxy)-6-methoxyquinazoline;
(83) 4-[(3-ethynylphenyl)amino]-7-({1-[(ethoxycarbonyl)methyl]piperidin-4-yl}methoxy)-6-methoxyquinazoline;
(84) 4-[(3-bromophenyl)amino]-7-({1-[(ethoxycarbonyl)methyl]piperidin-4-yl}methoxy)-6-methoxyquinazoline;
(85) 4-[(3-bromophenyl)amino]-7-({1-[(hexyloxycarbonyl)methyl]piperidin-4-yl}methoxy)-6-methoxyquinazoline;
(86) 4-[(3-bromophenyl)amino]-7-({1-[2-(ethoxycarbonyl)ethyl]piperidin-4-yl}methoxy)-6-methoxyquinazoline;
(87) 4-[(3-bromophenyl)amino]-7-({1-[3-(ethoxycarbonyl)propyl]piperidin-4-yl}methoxy)-6-methoxyquinazoline;
(88) 4-[(3-bromophenyl)amino]-7-({1-[(ethoxycarbonyl)methyl]piperidin-3-yl}methoxy)-6-methoxyquinazoline;
(89) 4-[(3-bromophenyl)amino]-7-({1-[(ethoxycarbonyl)methyl]pyrrolidin-2-yl}methoxy)-6-methoxyquinazoline;
(90) 4-[(3-bromophenyl)amino]-7-({1-[(dimethoxyphosphoryl)methyl]piperidin-4-yl}methoxy)-6-methoxyquinazoline;
(91) 4-[(3-bromophenyl)amino]-7-[(1-{[(methoxy)(methyl)phosphoryl]methyl}piperidin-4-yl)methoxy]-6-methoxyquinazoline;
(92) 4-[(3-bromophenyl)amino]-7-({1-[1,2-bis(ethoxycarbonyl)ethyl]piperidin-4-yl}methoxy)-6-methoxyquinazoline;
(93) 4-[(3-bromophenyl)amino]-7-[(1-{1-[(ethoxycarbonyl)methyl]-2-(ethoxycarbonyl)ethyl}piperidin-4-yl)methoxy]-6-methoxyquinazoline;
(94) 4-[(3-bromophenyl)amino]-7-(2-{1-[1-(methoxycarbonyl)ethyl]piperidin-4-yl}ethoxy)-6-methoxyquinazoline;
(95) 4-[(3-bromophenyl)amino]-7-(2-{1-[(methoxycarbonyl)methyl]piperidin-4-yl}ethoxy)-6-methoxyquinazoline;
(96) 4-[(3-bromophenyl)amino]-7-(2-{4-[(methoxycarbonyl)methyl]piperazin-1-yl}ethoxy-6-methoxyquinazoline;
(97) 4-[(3-bromophenyl)amino]-7-(2-{4-[(ethoxycarbonyl)methyl]piperazin-1-yl}ethoxy)-6-methoxyquinazoline;
(98) 4-[(3-bromophenyl)amino]-7-(2-{1-[(ethoxycarbonyl)methyl]piperidin-4-yl}ethoxy)-6-methoxyquinazoline;
(99) 4-[(3-bromophenyl)amino]-7-(2-{1-[1,2-bis(ethoxycarbonyl)ethyl]piperidin-4-yl}ethoxy)-6-methoxyquinazoline;
(100) 4-[(3-bromophenyl)amino]-7-(2-{4-[1,2-bis(ethoxycarbonyl)ethyl]piperazin-1-yl}ethoxy)-6-methoxyquinazoline;
(101) 4-[(3-bromophenyl)amino]-7-[2-(4-{1-[(ethoxycarbonyl)methyl]-2-(ethoxycarbonyl)ethyl}piperazin-1-yl)ethoxy]-6-methoxyquinazoline;
(102) 4-[(3-bromophenyl)amino]-7-[2-(1-{1-[(ethoxycarbonyl)methyl]-2-(ethoxycarbonyl)ethyl}piperidin-4-yl)ethoxy]-6-methoxyquinazoline;
(103) 4-[(3-bromophenyl)amino]-7-{2-[2-(methoxycarbonyl)pyrrolidin-1-yl]ethoxy}-6-methoxyquinazoline;
(104) 4-[(3-bromophenyl)amino]-7-{2-[2-(ethoxycarbonyl)piperidin-1-yl]ethoxy}-6-methoxyquinazoline;
(105) 4-[(3-bromophenyl)amino]-7-(3-{1-[(methoxycarbonyl)methyl]piperidin-4-yl}propyloxy)-6-methoxyquinazoline;
(106) 4-[(3-bromophenyl)amino]-7-(3-{4-[(methoxycarbonyl)methyl]piperazin-1-yl}propyloxy)-6-methoxyquinazoline;
(107) 4-[(3-bromophenyl)amino]-7-(3-{4-[(ethoxycarbonyl)methyl]piperazin-1-yl}propyloxy)-6-methoxyquinazoline;
(108) 4-[(3-bromophenyl)amino]-7-(3-{1-[(ethoxycarbonyl)methyl]piperidin-4-yl}propyloxy)-6-methoxyquinazoline;
(109) 4-[(3-bromophenyl)amino]-7-(3-{1-[(ethoxycarbonyl)methyl]piperidin-4-yl}-2-hydroxypropyloxy)-6-methoxyquinazoline;
(110) 4-[(3-bromophenyl)amino]-7-(3-{4-[(ethoxycarbonyl)methyl]piperazin-1-yl}-2- hydroxypropyloxy)-6-methoxyquinazoline;
(111) 4-[(3-methylphenyl)amino]-7-(3-{4-[(ethoxycarbonyl)methyl]piperazin-1-yl}propyloxy)-6-methoxyquinazoline;
(112) 4-[(3-chlorophenyl)amino]-7-(3-{4-[(ethoxycarbonyl)methyl]piperazin-1-yl}propyloxy)-6-methoxyquinazoline;
(113) 4-[(indol-5-yl)amino]-7-(3-{4-[(ethoxycarbonyl)methyl]piperazin-1-yl}propyloxy)-6-methoxyquinazoline;
(114) 4-[(1-phenylethyl)amino]-7-(3-{4-[(ethoxycarbonyl)methyl]piperazin-1-yl}propyloxy)-6-methoxyquinazoline;
(115) 4-[(3-bromophenyl)amino]-7-{3-[2-(methoxycarbonyl)pyrrolidin-1-yl]propyloxy}-6-methoxyquinazoline;
(116) 4-[(3-bromophenyl)amino]-7-{3-[3-(methoxycarbonyl)-4-methyl-piperazin-1-yl]propyloxy}-6-methoxyquinazoline;
(117) 4-[(3-bromophenyl)amino]-7-({1-[(ethoxycarbonyl)methyl]piperidin-4-yl}methoxy)-6-ethoxyquinazoline;
(118) 4-[(3-bromophenyl)amino]-7-({1-[(ethoxycarbonyl)methyl]piperidin-4-yl}methoxy)-6-(2-methoxyethoxy)quinazoline;
(119) 4-[(3-bromophenyl)amino]-7-(2-{1-[(ethoxycarbonyl)methyl]piperidin-4-yl}ethoxy)-6-(2-methoxyethoxy)quinazoline;
(120) 4-[(3-bromophenyl)amino]-7-(2-{4-[(ethoxycarbonyl)methyl]piperazin-1-yl}ethoxy)-6-(2-methoxyethoxy)quinazoline;
(121) 4-[(3-bromophenyl)amino]-7-(2-{4-[(ethoxycarbonyl)methyl]piperazin-1-yl}ethoxy)-6-ethoxyquinazoline;
(122) 4-[(3-bromophenyl)amino]-7-(3-{1-[(ethoxycarbonyl)methyl]piperidin-4-yl}propyloxy)-6-ethoxyquinazoline;
(123) 4-[(3-bromophenyl)amino]-7-(3-{4-[(ethoxycarbonyl)methyl]piperazin-1-yl}propyloxy)-6-(2-methoxyethoxy)quinazoline;
(124) 4-[(3-bromophenyl)amino]-7-(3-{1-[(dimethoxyphosphoryl)methyl]piperidin-4-yl}propyloxy)-6-methoxyquinazoline;
(125) 4-[(3-bromophenyl)amino]-7-(3-{4-[(dimethoxyphosphoryl)methyl]piperazin-1-yl}propyloxy)-6-methoxyquinazoline;
(126) 4-[(3-bromophenyl)amino]-7-[3-(4-{[(methoxy)(ethyl)phosphoryl]methyl}piperazin-1-yl)propyloxy]-6-methoxyquinazoline;
(127) 4-[(3-bromophenyl)amino]-7-[3-(1-{[(methoxy)(ethyl)phosphoryl]methyl}piperidin-4-yl)propyloxy]-6-methoxyquinazoline;
(128) 4-[(3-bromophenyl)amino]-7-(3-{4-[1,2-bis(ethoxycarbonyl)ethyl]piperazin-1-yl}propyloxy)-6-methoxyquinazoline;
(129) 4-[(3-bromophenyl)amino]-7-[3-(1-{1-[(ethoxycarbonyl)methyl]-2-(ethoxycarbonyl)ethyl}piperidin-4-yl)propyloxy]-6-methoxyquinazoline;
(130) 4-[(3-bromophenyl)amino]-7-(4-{1-[(ethoxycarbonyl)methyl]piperidin-4-yl}butyloxy)-6-methoxyquinazoline;
(131) 4-[(3-bromophenyl)amino]-7-(4-{4-[(ethoxycarbonyl)methyl]piperazin-1-yl}butyloxy)-6-methoxyquinazoline;
(132) 4-[(3-bromophenyl)amino]-7-(2-{N-[(ethoxycarbonyl)methyl]-N-methylamino}ethoxy)-6-methoxyquinazoline;
(133) 4-[(3-bromophenyl)amino]-7-(2-{N,N-bis[(ethoxycarbonyl)methyl]amino}ethoxy)-6-methoxyquinazoline;
(134) 4-[(3-bromophenyl)amino]-7-(2-{N-[(ethoxycarbonyl)methyl]-N-ethylamino}ethoxy)-6-methoxyquinazoline;
(135) 4-[(3-bromophenyl)amino]-7-(2-{N-[(ethoxycarbonyl)methyl]-N-(cyclopropylmethyl)amino}ethoxy)-6-methoxyquinazoline;
(136) 4-[(3-bromophenyl)amino]-7-(2-{[(ethoxycarbonyl)methyl]amino}ethoxy)-6-methoxyquinazoline;
(137) 4-[(3-bromophenyl)amino]-7-(2-{N-[(ethoxycarbonyl)methyl]-N-cyclopropylamino}ethoxy)-6-methoxyquinazoline;
(138) 4-[(3-bromophenyl)amino]-7-(2-{N-[(methoxycarbonyl)methyl]-N-methylamino}ethoxy)-6-methoxyquinazoline;
(139) 4-[(3-bromophenyl)amino]-7-(3-{N-[(methoxycarbonyl)methyl]-N-methylamino}propyloxy)-6-methoxyquinazoline;
(140) 4-[(3-bromophenyl)amino]-7-(3-{N,N-bis[(methoxycarbonyl)methyl]amino}propyloxy)-6-methoxyquinazoline;
(141) 4-[(3-bromophenyl)amino]-7-(3-{[(ethoxycarbonyl)methyl]amino}propyloxy)-6-methoxyquinazoline;
(142) 4-[(3-bromophenyl)amino]-7-(4-{N-[(ethoxycarbonyl)methyl]-N-methylamino}butyloxy)-6-methoxyquinazoline;
(143) 4-[(3-bromophenyl)amino]-7-(4-{N,N-bis[(ethoxycarbonyl)methyl]amino}butyloxy)-6-methoxyquinazoline;
(144) 4-[(3-bromophenyl)amino]-7-({4-[(methoxycarbonyl)methyl]-2-oxomorpholin-6-yl}methyloxy)-6-methoxyquinazoline;
(145) 4-[(3-bromophenyl)amino]-7-[(4-methyl-2-oxomorpholin-6-yl)methyloxy]-6-methoxyquinazoline;
(146) 4-[(3-bromophenyl)amino]-7-[(2-oxomorpholin-6-yl)methyloxy]-6-methoxyquinazoline;
(147) 4-[(4-amino-3,5-dibromophenyl)amino]-7-(3-{4-[(methoxycarbonyl)methyl]piperazin-1-yl}propyloxy)-6-methoxyquinazoline;
(148) 4-[(4-amino-3,5-dibromophenyl)amino]-7-(3-{1-[(methoxycarbonyl)methyl]piperidin-4-yl}propyloxy)-6-methoxyquinazoline;
(149) 4-[(3-bromophenyl)amino]-7-[3-(morpholino)propyloxy]-6-[(ethoxycarbonyl)methoxy]quinazoline;
(150) 4-[(3-bromophenyl)amino]-7-[2-(morpholino)ethoxy]-6-[(ethoxycarbonyl)methoxy]quinazoline;
(151) 4-[(3-bromophenyl)amino]-6-[2-(2-oxomorpholin-4-yl)ethoxy]-7-methoxyquinazoline;
(152) 4-[(3-bromophenyl)amino]-6-[3-(2-oxomorpholin-4-yl)propyloxy]-7-methoxyquinazoline;
(153) 4-[(3-bromophenyl)amino]-6-[2-(3-methyl-2-oxomorpholin-4-yl)ethoxy]-7-methoxyquinazoline;
(154) 4-[(3-bromophenyl)amino]-6-[2-(5,5-dimethyl-2-oxomorpholin-4-yl)ethoxy]-7-methoxyquinazoline;
(155) 4-[(3-bromophenyl)amino]-6-(2-{4-[(ethoxycarbonyl)methyl]piperazin-1-yl}ethoxy)-7-cyclopropylmethoxyquinazoline;
(156) 4-[(3-bromophenyl)amino]-6-(2-{4-[(ethoxycarbonyl)methyl]piperazin-1-yl}ethoxy)-7-cyclobutyloxyquinazoline;
(157) 4-[(3-bromophenyl)amino]-6-(2-{4-[(ethoxycarbonyl)methyl]piperazin-1-yl}ethoxy)-7-cyclopentyloxyquinazoline;
(158) 4-[(3-bromophenyl)amino]-6-(2-{4-[(ethoxycarbonyl)methyl]piperazin-1-yl}ethoxy)-7-cyclohexyloxyquinazoline;
(159) 4-[(3-bromophenyl)amino]-6-(2-{4-[(ethoxycarbonyl)methyl]piperazin-1-yl}ethoxy)-7-cyclopentylmethoxyquinazoline;
(160) 4-[(3-bromophenyl)amino]-6-(2-{4-[(ethoxycarbonyl)methyl]piperazin-1-yl}ethoxy)-7-cyclohexylmethoxyquinazoline;
(161) 4-[(3-bromophenyl)amino]-6-(2-{4-[(benzyloxycarbonyl)methyl]piperazin-1-yl}ethoxy)-7-methoxyquinazoline;
(162) 4-[(3-bromophenyl)amino]-6-(2-{4-[(phenyloxycarbonyl)methyl]piperazin-1-yl}ethoxy)-7-methoxyquinazoline;
(163) 4-[(3-bromophenyl)amino]-6-(2-{4-[(indan-5-yloxycarbonyl)methyl]piperazin-1-yl}ethoxy)-7-methoxyquinazoline;
(164) 4-[(3-bromophenyl)amino]-6-(2-{4-[(cyclohexyloxycarbonyl)methyl]piperazin-1-yl}ethoxy)-7-methoxyquinazoline;
(165) 4-[(3-bromophenyl)amino]-6-(2-{4-[(cyclohexylmethoxycarbonyl)methyl]piperazin-1-yl}ethoxy)-7-methoxyquinazoline;
(166) 4-[(3-bromophenyl)amino]-6-cyclopropylmethoxy-7-(2-{4-[(ethoxycarbonyl)methyl]piperazin-1-yl}ethoxy)quinazoline;
(167) 4-[(3-bromophenyl)amino]-6-cyclobutyloxy-7-(2-{4-[(ethoxycarbonyl)methyl]piperazin-1-yl}ethoxy)quinazoline;
(168) 4-[(3-bromophenyl)amino]-6-cyclopentyloxy-7-(2-{4-[(ethoxycarbonyl)methyl]piperazin-1-yl}ethoxy)quinazoline;
(169) 4-[(3-bromophenyl)amino]-6-cyclopentylmethoxy-7-(2-{4-[(ethoxycarbonyl)methyl]piperazin-1-yl}ethoxy)quinazoline;
(170) 4-[(3-bromophenyl)amino]-6-cyclohexylmethoxy-7-(2-{4-[(ethoxycarbonyl)methyl]piperazin-1-yl}ethoxy)quinazoline;
(171) 4-[(3-bromophenyl)amino]-6-cyclohexyloxy-7-(2-{4-[(ethoxycarbonyl)methyl]piperazin-1-yl}ethoxy)quinazoline;
(172) 4-[(3-chloro-4-fluorophenyl)amino]-6-[2-(6,6-dimethyl-2-oxomorpholin-4-yl)ethoxy]-7-methoxyquinazoline;
(173) 4-[(3-chloro-4-fluorophenyl)amino]-6-[2-(6,6-dimethyl-2-oxomorpholin-4-yl)ethoxy]-7-cyclobutyloxyquinazoline;
(174) 4-[(3-chloro-4-fluorophenyl)amino]-6-[2-(6,6-dimethyl-2-oxomorpholin-4-yl)ethoxy]-7-cyclopentyloxyquinazoline;
(175) 4-[(3-chloro-4-fluorophenyl)amino]-6-[2-(6,6-dimethyl-2-oxomorpholin-4-yl)ethoxy]-7-cyclohexyloxyquinazoline;
(176) 4-[(3-chloro-4-fluorophenyl)amino]-6-[2-(6,6-dimethyl-2-oxomorpholin-4-yl)ethoxy]-7-cyclopropylmethoxyquinazoline;
(177) 4-[(3-chloro-4-fluorophenyl]amino]-6-[2-(6,6-dimethyl-2-oxomorpholin-4-yl)ethoxy]-7-cyclopentylmethoxyquinazoline;
(178) 4-[(3-chloro-4-fluorophenyl)amino]-6-[2-(6,6-dimethyl-2-oxomorpholin-4-yl)ethoxy]-7-cyclohexylmethoxyquinazoline;
(179) 4-[(3-chloro-4-fluorophenyl)amino]-6-{2-[N-(2-oxotetrahydrofuran-4-yl)-N-methylamino]ethoxy}-7-methoxyquinazoline;
(180) 4-[(3-chloro-4-fluorophenyl)amino]-6-{2-[N-(2-oxotetrahydrofuran-4-yl)-N-methylamino]ethoxy}-7-cyclopentyloxyquinazoline;
(181) 4-[(3-chloro-4-fluorophenyl)amino]-6-{2-[N-(2-oxotetrahydrofuran-4-yl)-N-methylamino]ethoxy}-7-cyclopentylmethoxyquinazoline;
(182) 4-[(3-chloro-4-fluorophenyl)amino]-6-{2-[N-(2-oxotetrahydrofuran-3-yl)-N-methylamino]ethoxy}-7-methoxyquinazoline;
(183) 4-[(3-chloro-4-fluorophenyl)amino]-6-{2-[N-(2-oxotetrahydrofuran-3-yl)-N-methylamino]ethoxy}-7-cyclopentyloxyquinazoline;
(184) 4-[(3-chloro-4-fluorophenyl)amino]-6-{2-[N-(2-oxotetrahydrofuran-3-yl)-N-methylamino]ethoxy}-7-cyclopentylmethoxyquinazoline;
(185) 4-[(3-chloro-4-fluorophenyl)amino]-6-[3-(6,6-dimethyl-2-oxomorpholin-4-yl)propyloxy]-7-methoxyquinazoline;
(186) 4-[(3-chloro-4-fluorophenyl)amino]-6-[3-(6,6-dimethyl-2-oxomorpholin-4-yl)propyloxy]-7-cyclopentyloxyquinazoline;
(187) 4-[(3-chloro-4-fluorophenyl)amino]-6-[3-(6,6-dimethyl-2-oxomorpholin-4-yl)propyloxy]-7-cyclopentylmethoxyquinazoline;
(188) (R)-4-[(1phenylethyl)amino]-6-[3-(6,6-dimethyl-2-oxomorpholin-4-yl)propyloxy]-7-cyclopentyloxyquinazoline;
(189) 4-[(3-chloro-4-fluorophenyl)amino]-7-[2-(6,6-dimethyl-2-oxomorpholin-4-yl)ethoxy]-6-methoxyquinazoline;
(190) 4-[(3-chloro-4-fluorophenyl)amino]-7-[2-(6,6-dimethyl-2-oxomorpholin-4-yl)ethoxy]-6-cyclobutyloxyquinazoline;
(191) 4-[(3-chloro-4-fluorophenyl)amino]-7-[2-(6,6-dimethyl-2-oxomorpholin-4-yl)ethoxy]-6-cyclopentyloxyquinazoline;
(192) 4-[(3-chloro-4-fluorophenyl)amino]-7-[2-(6,6-dimethyl-2-oxomorpholin-4-yl)ethoxy]-6-cyclohexyloxyquinazoline;
(193) 4-[(3-chloro-4-fluorophenyl)amino]-7-[2-(6,6-dimethyl-2-oxomorpholin-4-yl)ethoxy]-6-cyclopropylmethoxyquinazoline;
(194) 4-[(3-chloro-4-fluorophenyl]amino]-7-[2-(6,6-dimethyl-2-oxomorpholin-4-yl)ethoxy]-6-cyclopentylmethoxyquinazoline;
(195) 4-[(3-chloro-4-fluorophenyl)amino]-7-[2-(6,6-dimethyl-2-oxomorpholin-4-yl)ethoxy]-6-cyclohexylmethoxyquinazoline;
(196) 4-[(3-chloro-4-fluorophenyl)amino]-7-{2-[N-(2-oxotetrahydrofuran-4-yl)-N-methylamino]ethoxy}-6-methoxyquinazoline;
(197) 4-[(3-chloro-4-fluorophenyl)amino]-7-{2-[N-(2-oxotetrahydrofuran-4-yl)-N-methylamino]ethoxy}-6-cyclopentyloxyquinazoline;
(198) 4-[(3-chloro-4-fluorophenyl)amino]-7-{2-[N-(2-oxotetrahydrofuran-4-yl)-N-methylamino]ethoxy}-6-cyclopentylmethoxyquinazoline;
(199) 4-[(3-chloro-4-fluorophenyl)amino]-7-{2-[N-(2-oxotetrahydrofuran-3-yl)-N-methylamino]ethoxy}-6-methoxyquinazoline;
(200) 4-[(3-chloro-4-fluorophenyl)amino]-7-{2-[N-(2-oxotetrahydrofuran-3-yl)-N-methylamino]ethoxy}-6-cyclopentyloxyquinazoline;
(201) 4-[(3-chloro-4-fluorophenyl)amino]-7-{2-[N-(2-oxotetrahydrofuran-3-yl)-N-methylamino]ethoxy}-6-cyclopentylmethoxyquinazoline;
(202) 4-[(3-chloro-4-fluorophenyl)amino]-7-[3-(6,6-dimethyl-2-oxomorpholin-4-yl)propyloxy]-6-methoxyquinazoline;
(203) 4-[(3-chloro-4-fluorophenyl)amino]-7-[3-(6,6-dimethyl-2-oxomorpholin-4-yl)propyloxy]-6-cyclopentyloxyquinazoline;
(204) 4-[(3-chloro-4-fluorophenyl)amino]-7-[3-(6,6-dimethyl-2-oxomorpholin-4-yl)propyloxy]-6-cyclopentylmethoxyquinazoline; and

(205) (R)-4-[(1-phenylethyl)amino]-7-[3-(6,6-dimethyl-2-oxomorpholin-4-yl)propyloxy]-6-cyclopentyloxyquinazoline.

EXAMPLE 6
Coated Tablets Containing 75 mg of Active Substance

| Component | Amount per tablet core (mg) |
|---|---|
| active substance | 75 |
| calcium phosphate | 93.0 |
| corn starch | 35.5 |
| polyvinylpyrrolidone | 10.0 |
| hydroxypropylmethylcellulose | 15.0 |
| magnesium stearate | 1.5 |
| TOTAL | 230.0 |

Preparation:

The active substance is mixed with calcium phosphate, corn starch, polyvinylpyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape. Weight of core: 230 mg; die: 9 mm, convex. The tablet cores thus produced are coated with a film consisting essentially of hydroxypropylmethylcellulose. The finished film-coated tablets are polished with beeswax. Weight of coated tablet: 245 mg.

EXAMPLE 7
Tablets Containing 100 mg of Active Substance

| Component | Amount per tablet (mg) |
|---|---|
| active substance | 100.0 |
| lactose | 80.0 |
| corn starch | 34.0 |
| polyvinylpyrrolidone | 4.0 |
| magnesium stearate | 2.0 |
| TOTAL | 220.0 |

Preparation:

The active substance, lactose, and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets. Weight of tablet: 220 mg; diameter: 10 mm, biplanar, facetted on both sides and notched on one side.

EXAMPLE 8
Tablets Containing 150 mg of Active Substance

| Component | Amount per tablet (mg) |
|---|---|
| active substance | 150.0 |
| powdered lactose | 89.0 |
| corn starch | 40.0 |
| colloidal silica | 10.0 |
| polyvinylpyrrolidone | 10.0 |
| magnesium stearate | 1.0 |
| TOTAL | 300.0 |

Preparation:

The active substance mixed with lactose, corn starch, and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture. Weight of tablet: 300 mg; die: 10 mm, flat.

EXAMPLE 9
Hard Gelatine Capsules Containing 150 mg of Active Substance

| Component | Amount per capsule (mg) |
|---|---|
| active substance | 150.0 |
| corn starch (dried) | approx. 80.0 |
| lactose (powdered) | approx. 87.0 |
| magnesium stearate | 3.0 |
| TOTAL | 320.0 |

Preparation:

The active substance is mixed with the excipients, passed through a screen with a mesh size of 0.75 mm and homogeneously mixed using a suitable apparatus. The finished mixture is packed into size 1 hard gelatine capsules. Capsule filling: approx. 320 mg; capsule shell: size 1 hard gelatine capsule.

EXAMPLE 10
Suppositories Containing 150 mg of Active Substance

| Component | Amount per suppository (mg) |
|---|---|
| active substance | 150.0 |
| polyethyleneglycol 1500 | 550.0 |
| polyethyleneglycol 6000 | 460.0 |
| polyoxyethylene sorbitan monostearate | 840.0 |
| TOTAL | 2000.0 |

Preparation:

After the suppository mass has been melted the active substance is homogeneously distributed therein and the melt is poured into chilled molds.

EXAMPLE 11
Suspension Containing 50 mg of Active Substance/5 ml

| Component | Amount/100 ml suspension | |
|---|---|---|
| active substance | 1.0 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavoring | 0.30 | g |
| distilled water | ad 100 | ml |

Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavoring have been added and dissolved, the suspension is evacuated with stirring to eliminate air. 5 ml of suspension contains 50 mg of active substance.

EXAMPLE 12
Ampoules Containing 10 mg of Active Substance

| Component | Amount | |
|---|---|---|
| active substance | 10.0 | mg |
| 0.01N hydrochloric acid | q.s. | |
| double-distilled water | ad 2.0 | ml |

Preparation:

The active substance is dissolved in the necessary amount of 0.01 N HCl, made isotonic with common salt, filtered sterile and transferred into 2 ml ampoules.

EXAMPLE 13
Ampoules Containing 50 mg of Active Substance

| Component | Amount | |
|---|---|---|
| active substance | 50.0 | mg |
| 0.01N hydrochloric acid | q.s. | |
| double-distilled water | ad 10.0 | ml |

Preparation:

The active substance is dissolved in the necessary amount of 0.01 N HCl, made isotonic with common salt, filtered sterile and transferred into 10 ml ampoules.

EXAMPLE 14
Capsules for Powder Inhalation Containing 5 mg of Active Substance

| Component | Amount per capsule (mg) |
|---|---|
| active substance | 5.0 |
| lactose for inhalation | 15.0 |
| TOTAL | 20.0 |

Preparation:

The active substance is mixed with lactose for inhalation. The mixture is packed into capsules in a capsule-making machine (weight of the empty capsule approx. 50 mg). Weight of capsule: 70.0 mg; size of capsule: 3.

EXAMPLE 15
Solution for Inhalation for Hand-Held Nebulizers
Containing 2.5 mg of Active Substance

| Component | Amount per spray | |
|---|---|---|
| active substance | 2.500 | mg |
| benzalkonium chloride | 0.001 | mg |
| 1N hydrochloric acid | q.s. | |
| ethanol/water (50/50) | ad 15.000 | mg |

Preparation:

The active substance and benzalkonium chloride are dissolved in ethanol/water (50/50). The pH of the solution is adjusted with 1N hydrochloric acid. The resulting solution is filtered and transferred into suitable containers for use in hand-held nebulizers (cartridges). Contents of the container: 4.5 g

We claim:

1. A compound of formula (I) 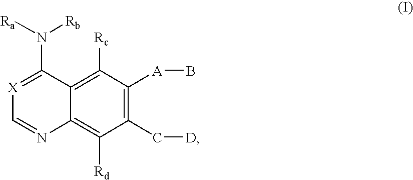
wherein:
Rₐ is a hydrogen atom or a C₁₋₄-alkyl group;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, bromine, or iodine atom,
(ii) a C₁₋₄-alkyl, hydroxy, C₁₋₄-alkoxy, C₃₋₆-cycloalkyl, C₄₋₆-cycloalkoxy, C₂₋₅-alkenyl, or C₂₋₅-alkynyl group,
(iii) an aryl, aryloxy, arylmethyl, or arylmethoxy group,
(iv) a C₃₋₅-alkenyloxy or C₃₋₅-alkynyloxy group, wherein the unsaturated moiety thereof is not linked to the oxygen atom,
(v) a C₁₋₄-alkylsulfenyl, C₁₋₄-alkylsulfinyl, C₁₋₄-alkylsulfonyl, C₁₋₄-alkylsulfonyloxy, trifluoromethylsulfenyl, trifluoromethylsulfinyl, or trifluoromethylsulfonyl group,
(vi) a methyl or methoxy group substituted by 1 to 3 fluorine atoms,
(vii) an ethyl or ethoxy group substituted by 1 to 5 fluorine atoms, or
(viii) a cyano or nitro group or an amino group optionally substituted by one or two C₁₋₄-alkyl groups, wherein the substituents are identical or different, or
R₁ together with R₂, if they are bound to adjacent carbon atoms, are a —CH═CH—CH═CH, —CH═CH—NH, or —CH═N—NH group, and
R₃ is a hydrogen, fluorine, chlorine, or bromine atom, or a C₁₋₄-alkyl, trifluoromethyl, or C₁₋₄-alkoxy group;
X is a methine group substituted by a cyano group or a nitrogen atom;
A is a group consisting of:
(a) —O—C₁₋₆-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom thereof in each case is linked to the bicyclic heteroaromatic moiety of formula (I),
(b) an —O—C₁₋₆-alkylene group substituted by an R₆O—CO or R₆O—CO—C₁₋₄-alkyl group, wherein the oxygen atom of the —O—C₁₋₆-alkylene group is linked to the bicyclic heteroaromatic moiety of formula (I),
(c) an —O—C₂₋₆-alkylene group substituted at a position other than position 1 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group, wherein the oxygen atom of the —O—C₂₋₆-alkylene group is linked to the bicyclic heteroaromatic moiety of formula (I),
(d) a —C₁₋₆-alkylene group,
(e) an —NR₄—C₁₋₆-alkylene, —NR—C₃₋₇-cycloalkylene, —N—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety thereof in each case is linked to the bicyclic heteroaromatic moiety of formula (I),
(f) an oxygen atom linked to a carbon atom of the group B, or
(g) a NR₄ group linked to a carbon atom of the group B,
B is a group consisting of:
(a) an R₆O—CO-alkylene-NR₅, (R₇O—PO—OR₈)-alkylene-NR₅, or (R₇O—PO—R₉)-alkylene-NR₅ group, wherein in each case the alkylene moiety, which is straight-chained and contains 1 to 6 carbon atoms, is additionally optionally substituted by one or two C₁₋₂-alkyl groups or by an R₆O—CO or R₆O—CO—C₁₋₂-alkyl group,
(b) a 4- to 7-membered alkyleneimino group substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(c) a 4- to 7-membered alkyleneimino group substituted by two R₆O—CO or R₆O—CO—C₁₋₄-alkyl groups or by an R₆OCO group and an R₆O—CO—C₁₋₄-alkyl group,
(d) a piperazino or homopiperazino group substituted in each case at the 4 position by R₁₀ and additionally substituted at a cyclic carbon atom thereof by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(e) a piperazino or homopiperazino group substituted in each case at the 4 position by R₁₀ and additionally substituted at cyclic carbon atoms thereof by two R₆O—CO or R₆O—CO—C₁₋₄-alkyl groups or by an R₆O—CO group and an R₆O—CO—C₁₋₄-alkyl group,
(f) a piperazino or homopiperazino group substituted in each case at the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(g) a piperazino or homopiperazino group substituted in each case at the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group and additionally substituted at cyclic carbon atoms thereof by one or two R₆O—CO or R₆O—CO—C₁₋₄-alkyl groups or by an R₆O—CO group and an R₆O—CO—C₁₋₄-alkyl group,
(h) a morpholino or homomorpholino group substituted in each case by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(i) a morpholino or homomorpholino group substituted in each case by two R₆O—CO or R₆O—CO—C₁₋₄-alkyl groups or by an R₆O—CO group and an R₆O—CO—C₁₋₄-alkyl group,
(j) a pyrrolidinyl, piperidinyl, or hexahydroazepinyl group substituted in each case at the 1 position by R₁₀, wherein the 5 to 7-membered rings thereof in each case are additionally substituted at a carbon atom thereof by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(k) a pyrrolidinyl, piperidinyl, or hexahydroazepinyl group substituted in each case at the 1 position by R₁₀, wherein the 5 to 7-membered rings thereof in each case are additionally substituted at carbon atoms thereof by two R₆O—CO or R₆O—CO—C₁₋₄-alkyl groups or by an R₆O—CO group and an R₆O—CO—C₁₋₄-alkyl group,
(l) a pyrrolidinyl, piperidinyl, or hexahydroazepinyl group substituted in each case at the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(m) a pyrrolidinyl, piperidinyl, or hexahydroazepinyl group substituted in each case at the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group, wherein the 5- to 7-membered rings thereof in each case are additionally substituted at carbon atoms thereof by one or two R₆O—CO or R₆O—CO—C₁₋₄-alkyl groups or by an R₆O—CO group and an R₆O—CO—C₁₋₄-alkyl group,
(n) a 2-oxomorpholino group substituted by 1 to 4 C₁₋₂-alkyl groups,
(o) a 2-oxomorpholinyl group substituted at the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group, wherein the 2-oxomorpholinyl group thereof is linked to a carbon atom of the group A, and
(p) an R₁₁NR₅ group, or
A together with B are a group consisting of:
(a) a hydrogen, fluorine, or chlorine atom,
(b) a C₁₋₆-alkoxy group,
(c) a C₂₋₆-alkoxy group substituted at a position other than position 1 by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, 4-(C₁₋₄-alkyl)-homopiperazino, or 1-imidazolyl group,
(d) a C₁₋₄-alkoxy group substituted by a pyrrolidinyl, piperidinyl, or hexahydroazepinyl group substituted at the 1 position by R₁₀,
(e) a C₁₋₆-alkoxy group substituted by an R₆O—CO, (R₇O—PO—OR₈), or (R₇O—PO—R₉) group,
(f) a C₃₋₇-cycloalkoxy or C₃₋₇-cycloalkyl-C₁₋₄-alkoxy group,
(g) an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group, and
(h) a 2-oxomorpholino group optionally substituted by 1 or 2 methyl groups;
C is a group consisting of:
(a) an —O—C₁₋₆-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom thereof in each case is linked to the bicyclic heteroaromatic moiety of formula (I),
(b) an —O—C₁₋₆-alkylene group substituted by an R₆O—CO or R₆O—CO—C₁₋₄-alkyl group, wherein the oxygen atom of the —O—C₁₋₆-alkylene group is linked to the bicyclic heteroaromatic moiety of formula (I),
(c) an —O—C₂₋₆-alkylene group substituted at a position other than position 1 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group and the oxygen atom of the —O—C₂₋₆-alkylene group is linked to the bicyclic heteroaromatic moiety of formula (I),
(d) a —C₁₋₆-alkylene group,
(e) an —NR₄—C₁₋₆-alkylene, —NR₄—C₃₋₇-cycloalkylene, —NR₄—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety thereof in each case is linked to the bicyclic heteroaromatic moiety of formula (I),
(f) an oxygen atom linked to a carbon atom of the group D, and
(g) a NR₄ group linked to a carbon atom of the group D,
D is a group consisting of:
(a) an R₆O—CO-alkylene-NR₅, (R₇O—PO—OR₈)-alkylene-NR₅, or (R₇O—PO—R₉)-alkylene-NR₅ group wherein in each case the alkylene moiety thereof, which is straight-chained and contains 1 to 6 carbon atoms, is additionally optionally substituted by one or two C₁₋₂-alkyl groups or by an R₆O—CO or R₆O—CO—C₁₋₂-alkyl group,
(b) a 4- to 7-membered alkyleneimino group substituted by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(c) a 4- to 7-membered alkyleneimino group substituted by two R₆O—CO or R₆O—CO—C₁₋₄-alkyl groups or by an R₆OCO group and an R₆O—CO—C₁₋₄-alkyl group,
(d) a piperazino or homopiperazino group in each case substituted at the 4 position by R₁₀ and additionally substituted at a cyclic carbon atom thereof by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(e) a piperazino or homopiperazino group in each case substituted at the 4 position by R₁₀ and additionally substituted at cyclic carbon atoms thereof by two R₆O—CO or R₆O—CO—C₁₋₄-alkyl groups or by an R₆O—CO group and an R₆O—CO—C₁₋₄-alkyl group,
(f) a piperazino or homopiperazino group substituted in each case at the 4 position by an R₆O—CO—C₁₋₄-alkyl, bis-(R₆—CO)—C₁₋₄-alkyl, (R₇O—PO—OR₈)—C₁₋₄-alkyl, or (R₇O—PO—R₉)—C₁₋₄-alkyl group,
(g) a piperazino or homopiperazino group substituted in each case at the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group and additionally substituted at cyclic carbon atoms thereof by one or two R₆O—CO or R₆O—CO—C₁₋₄-alkyl groups or by an R₆O—CO group and an R₆O—CO—C₁₋₄-alkyl group,
(h) a morpholino or homomorpholino group substituted in each case by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(i) a morpholino or homomorpholino group substituted in each case by two R₆O—CO or R₆O—CO—C₁₋₄-alkyl groups or by an R₆O—CO group and an R₆O—CO—C₁₋₄-alkyl group,
(j) a pyrrolidinyl, piperidinyl, or hexahydroazepinyl group substituted in each case at the 1 position by R₁₀, wherein the 5- to 7-membered rings thereof in each case are additionally substituted at a carbon atom by an R₆O—CO, (R₇O—PO—OR₈), (R₇O—PO—R₉), R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(k) a pyrrolidinyl, piperidinyl, or hexahydroazepinyl group substituted in each case at the 1 position by R₁₀, wherein the 5- to 7-membered rings thereof in each case are additionally substituted at carbon atoms thereof by two R₆O—CO or R₆O—CO—C₁₋₄-alkyl groups or by an R₆O—CO group and an R₆O—CO—C₁₋₄-alkyl group,
(l) a pyrrolidinyl, piperidinyl, or hexahydroazepinyl group substituted in each case at the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(m) a pyrrolidinyl, piperidinyl, or hexahydroazepinyl group substituted in each case at the 1 position by an R₇O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group, wherein the 5- to 7-membered rings thereof in each case are additionally substituted at carbon atoms thereof by one or two R₆O—CO or R₆O—CO—C₁₋₄-alkyl groups or by an R₆O—CO group and an R₆O—CO—C₁₋₄-alkyl group,
(n) a 2-oxomorpholino group optionally substituted by 1 to 4 C₁₋₂-alkyl groups,
(o) a 2-oxomorpholinyl group substituted at the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group, wherein the 2-oxomorpholinyl group is linked to a carbon atom of the group C, and
(p) an R₁₁NR₅ group, or
C and D together are a group consisting of:
(a) a hydrogen, fluorine, or chlorine atom;
(b) a C₁₋₆-alkoxy group,
(c) a C₂₋₆-alkoxy group substituted at a position other than position 1 by a hydroxy, C₁₋₄-alkoxy, amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, 4-(C₁₋₄-alkyl)-homopiperazino, or 1-imidazolyl group,
(d) a C₁₋₄-alkoxy group substituted by a pyrrolidinyl, piperidinyl, or hexahydroazepinyl group substituted at the 1 position by R₁₀,
(e) a C₁₋₆-alkoxy group substituted by an R₆O—CO, (R₇O—PO—OR₈), or (R₇O—PO—R₉) group,
(f) a C₃₋₇-cycloalkoxy or C₃₋₇-cycloalkyl-C₁₋₄-alkoxy group,
(g) an amino, C₁₋₄-alkylamino, di-(C₁₋₄-alkyl)-amino, pyrrolidino, piperidino, hexahydroazepino, morpholino, homomorpholino, piperazino, 4-(C₁₋₄-alkyl)-piperazino, homopiperazino, or 4-(C₁₋₄-alkyl)-homopiperazino group, and
(h) a 2-oxomorpholino group optionally substituted by 1 or 2 methyl groups,
with the proviso that:
(i) at least one of the groups B or D, or A together with B, or C together with D contains an optionally substituted 2-oxomorpholinyl group, an (R₇O—PO—OR₈) or (R₇O—PO—R₉) group, or
(ii) that at least one of the groups B or D contains an optionally substituted 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group, or
(iii) that at least one of the groups A, B, C, or D, or A together with B, or C together with D contains an R₆O—CO group and additionally one of the groups A, B, C, or D, or A together with B, or C together with D contains a primary, secondary, or tertiary amino function, wherein the nitrogen atom of this amino function is not linked to a carbon atom of an aromatic group,
R_{c} and R_{d}, which are identical or different, each are a hydrogen, fluorine, or chlorine atom, or a methoxy group or a methyl group optionally substituted by a methoxy, dimethylamino, diethylamino, pyrrolidino, piperidino, or morpholino group;
Rₑ and R_{f}, which are identical or different, in each case are a hydrogen atom or a C₁₋₄-alkyl group;
R_{g} is a C₁₋₄-alkyl, C₃₋₇-cycloalkyl, C₁₋₄-alkoxy, or C₅₋₇-cycloalkoxy group;
R₄ is a hydrogen atom or a C₁₋₄-alkyl group;
R₅ is a hydrogen atom,
a C₁₋₄-alkyl group optionally substituted by an R₆O—CO, (R₇O—PO—OR₈), or (R₇O—PO—R₆) group,
a C₂₋₄-alkyl group substituted at a position other than position 1 by a hydroxy, C₁₋₄-alkoxy, amino, C₁₋₄-alkylamino, or di-(C₁₋₄-alkyl)-amino group, or by a 4- to 7-membered alkyleneimino group, wherein a methylene group at the 4 position of the 6- to 7-membered alkyleneimino group is optionally replaced by an oxygen or sulfur atom, or by a sulfinyl, sulfonyl, imino, or N—(C₁₋₄-alkyl)-imino group, or
a C₃₋₇-cycloalkyl or C₃₋₇-cycloalkyl-C₁₋₃-alkyl group;
R₆, R₇, and R₈, which are identical or different, in each case are a hydrogen atom,
a C₁₋₈-alkyl group optionally substituted at a position other than position 1 by a hydroxy, C₁₋₄-alkoxy, amino, C₁₋₄-alkylamino, or di-(C₁₋₄-alkyl)-amino group or by a 4- to 7-membered alkyleneimino group, wherein a methylene group at the 4 position of the 6- to 7-membered alkyleneimino group is optionally replaced by an oxygen or sulfur atom, or by a sulfinyl, sulfonyl, imino, or N—(C₁₋₄-alkyl)-imino group,
a C₄₋₇-cycloalkyl group optionally substituted by 1 or 2 methyl groups,
a C₃₋₅-alkenyl or C₃₋₅-alkynyl group, wherein the unsaturated moiety thereof is not linked to the oxygen atom, or
a C₃₋₇-cycloalkyl-C₁₋₄-alkyl, aryl, aryl-C₁₋₄-alkyl, or R₆CO—O—(RₑCR_{f}) group;
R₉ is a C₁₋₄-alkyl, aryl, or aryl-C₁₋₄-alkyl group;
R₁₀ is a hydrogen atom, or a C₁₋₄-alkyl, formyl, C₁₋₄-alkylcarbonyl, or C₁₋₄-alkylsulfonyl group;
R₁₁ is a 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group each optionally substituted by one or two methyl groups;
R₁₂ is a cyano, carboxy, C₁₋₄-alkoxycarbonyl, aminocarbonyl, C₁₋₄-alkylaminocarbonyl, di-(C₁₋₄-alkyl)-aminocarbonyl, C₁₋₄-alkylsulfenyl, C₁₋₄-alkylsulfinyl, C₁₋₄-alkylsulfonyl, hydroxy, C₁₋₄-alkylsulfonyloxy, trifluoromethyloxy, nitro, amino, C₁₋₄-alkylamino, di-(C₁₋₄-alkyl)-amino, C₁₋₄-alkylcarbonylamino, N—(C₁₋₄-alkyl)-C₁₋₄-alkylcarbonylamino, C₁₋₄-alkylsulfonylamino, N—(C₁₋₄-alkyl)-C₁₋₄-alkylsulfonylamino, aminosulfonyl, C₁₋₄-alkylaminosulfonyl, or di-(C₁₋₄-alkyl)-aminosulfonyl group or a carbonyl group substituted by a 5- to 7-membered alkyleneimino group, wherein a methylene group at the 4 position of the 6- to 7-membered alkyleneimino group is optionally replaced by an oxygen or sulfur atom, or by a sulfinyl, sulfonyl, imino, or N—(C₁₋₄-alkyl)-imino group; and
R₁₃ is a fluorine, chlorine, bromine, or iodine atom, or a C₁₋₄-alkyl, trifluoromethyl, or C₁₋₄-alkoxy group, or two groups R₁₃, if they are bound to adjacent carbon atoms, together are a C₃₋₅-alkylene, methylenedioxy, or 1,3-butadien-1,4-ylene group,
wherein the aryl moieties of the abovementioned groups are identical or different phenyl groups optionally monosubstituted by R₁₂, mono-, di-, or trisubstituted by R₁₃, or monosubstituted by R₁₂ and additionally mono- or disubstituted by R₁₃, wherein the substituents are identical or different,
or a tautomer, stereoisomer, or salt thereof.

2. The compound of formula (I) according to claim 1, wherein Rₐ is a hydrogen atom.

3. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, bromine, or iodine atom;
(ii) a methyl, ethyl, hydroxy, methoxy, ethoxy, amino, cyano, vinyl, or ethynyl group,
(iii) an aryl, aryloxy, arylmethyl, or arylmethoxy group, or
(iv) a methyl or methoxy group substituted by 1 to 3 fluorine atoms, or R₁ together with R₂, if they are bound to adjacent carbon atoms, are a —CH═CH—CH═CH, —CH═CH—NH, or —CH═N—NH group, and
R₃ is a hydrogen, fluorine, chlorine, or bromine atom;
X is a nitrogen atom;
A is group consisting of:
(a) an —O—C₁₋₄-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom thereof in each case is linked to the bicyclic heteroaromatic moiety of formula (I),
(b) an —O—C₂₋₄-alkylene group substituted at a position other than position 1 by a hydroxy group, wherein the oxygen atom of the —O—C₂₋₄-alkylene group is linked to the bicyclic heteroaromatic moiety of formula (I), or
(c) an oxygen atom linked to a carbon atom of the group B,
B is a group consisting of:
(a) an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, is additionally optionally substituted by one or two C₁₋₂-alkyl groups or by an R₆O—CO or R₆O—CO—C₁₋₂-alkyl group,
(b) a 4- to 7-membered alkyleneimino group substituted by an R₆O—CO, R₆O—CO—C₁₋₄-alkyl, or bis-(R₆O—CO)—C₁₋₄-alkyl group,
(c) a 4- to 7-membered alkyleneimino group substituted by two R₆O—CO or R₆O—CO—C₁₋₄-alkyl groups,
(d) a piperazino or homopiperazino group substituted at the 4 position by R₁₀ and additionally substituted at a cyclic carbon atom thereof by an R₆O—CO, R₆O—CO—C₁₋₄-alkyl, or bis-(R₆O—CO)—C₁₋₄-alkyl group,
(e) a piperazino or homopiperazino group substituted at the 4 position by R₁₀ and additionally substituted at cyclic carbon atoms thereof by two R₆O—CO or R₆O—CO—C₁₋₄-alkyl groups,
(f) a piperazino or homopiperazino group which in each case is substituted at the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(g) a piperazino or homopiperazino group substituted in each case at the 4 position by an R₆O—CO—C₁₋₄-alkyl or bis-(R₆O—CO)—C₁₋₄-alkyl group and additionally substituted at cyclic carbon atoms thereof by one or two R₆O—CO or R₆O—CO—C₁₋₄-alkyl groups,
(h) a morpholino or homomorpholino group substituted in each case by an R₆O—CO, R₆O—CO—C₁₋₄-alkyl, or bis-(R₆O—CO)—C₁₋₄-alkyl group,
(i) a morpholino or homomorpholino group substituted in each case by two R₆O—CO or R₆O—CO—C₁₋₄-alkyl groups,
(j) a pyrrolidinyl, piperidinyl, or hexahydroazepinyl group substituted in each case at the 1 position by R₁₀, wherein the 5- to 7-membered rings thereof in each case are additionally substituted at a carbon atom thereof by an R₆O—CO, R₆O—CO—C₁₋₄-alkyl, or bis-(R₆O—CO)—C₁₋₄-alkyl group,
(k) a pyrrolidinyl, piperidinyl, or hexahydroazepinyl group substituted in each case at the 1 position by R₁₀, wherein the 5- to 7-membered rings thereof in each case are additionally substituted at carbon atoms thereof by two R₆O—CO or R₆O—CO—C₁₋₄-alkyl groups,
(l) a pyrrolidinyl, piperidinyl, or hexahydroazepinyl group substituted in each case at the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(m) a pyrrolidinyl, piperidinyl, or hexahydroazepinyl group substituted in each case at the 1 position by an R₆O—CO—C₁₋₄-alkyl or bis-(R₆O—CO)—C₁₋₄-alkyl group, wherein the 5- to 7-membered rings thereof in each case are additionally substituted at carbon atoms thereof by one or two R₆O—CO or R₆O—CO—C₁₋₄-alkyl groups,
(n) a 2-oxomorpholino group optionally substituted by 1 to 4 C₁₋₂-alkyl groups,
(o) a 2-oxomorpholinyl group substituted at the 4 position by a hydrogen atom, or by a C₁₋₄-alkyl or R₆O—CO—C₁₋₄-alkyl group, wherein the 2-oxomorpholinyl group is linked to a carbon atom of the group A, and
(p) an R₁₁NR₅ group, or
A together with B are a group consisting of:
(a) a hydrogen atom,
(b) a C₁₋₄-alkoxy group,
(c) a C₂₋₄-alkoxy group substituted at a position other than position 1 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group,
(d) a C₁₋₄-alkoxy group substituted by a pyrrolidinyl or piperidinyl group substituted at the 1 position by R₁₀,
(e) a C₁₋₄-alkoxy group substituted by an R₆O—CO group, and
(f) a C₄₋₇-cycloalkoxy or C₃₋₇-cycloalkyl-C₁₋₄-alkoxy group;
C is a group consisting of:
(a) an —O—C₁₋₄-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom thereof in each case is linked to the bicyclic heteroaromatic moiety of formula (I),
(b) an —O—C₂₋₄-alkylene group substituted at a position other than position 1 by a hydroxy group, wherein the oxygen atom of the —O—C₂₋₄-alkylene group is linked to the bicyclic heteroaromatic moiety of formula (I), and
(c) an oxygen atom linked to a carbon atom of the group D,
D is a group consisting of:
(a) an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, is additionally optionally substituted by one or two C₁₋₂-alkyl groups or by an R₆O—CO or R₆O—CO—C₁₋₂-alkyl group,
(b) a 4- to 7-membered alkyleneimino group substituted by an R₆O—CO, R₆O—CO—C₁₋₄-alkyl, or bis-(R₆O—CO)—C₁₋₄-alkyl group,
(c) a 4- to 7-membered alkyleneimino group substituted by two R₆O—CO or R₆O—CO—C₁₋₄-alkyl groups,
(d) a piperazino or homopiperazino group substituted at the 4 position by R₁₀ and additionally at a cyclic carbon atom thereof by an R₆O—CO, R₆O—CO—C₁₋₄-alkyl, or bis-(R₆O—CO)—C₁₋₄-alkyl group,
(e) a piperazino or homopiperazino group substituted at the 4 position by R₁₀ and additionally substituted at cyclic carbon atoms thereof by two R₆O—CO or R₆O—CO—C₁₋₄-alkyl groups,
(f) a piperazino or homopiperazino group substituted in each case at the 4 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(g) a piperazino or homopiperazino group substituted at the 4 position by an R₆O—CO—C₁₋₄-alkyl or bis-(R₆O—CO)—C₁₋₄-alkyl group and additionally substituted at cyclic carbon atoms thereof by one or two R₆O—CO or R₆O—CO—C₁₋₄-alkyl groups,
(h) a morpholino or homomorpholino group substituted in each case by an R₆O—CO, R₆O—CO—C₁₋₄-alkyl, or bis-(R₆O—CO)—C₁₋₄-alkyl group,
(i) a morpholino or homomorpholino group substituted by two R₆O—CO or R₆O—CO—C₁₋₄-alkyl groups,
(l) a pyrrolidinyl, piperidinyl, or hexahydroazepinyl group substituted at the 1 position by R₁₀, wherein the 5- to 7-membered rings thereof in each case are additionally substituted at a carbon atom thereof by an R₆O—CO, R₆O—CO—C₁₋₄-alkyl, or bis-(R₆O—CO)—C₁₋₄-alkyl group,
(k) a pyrrolidinyl, piperidinyl, or hexahydroazepinyl group substituted at the 1 position by R₁₀, wherein the 5- to 7-membered rings thereof in each case are additionally substituted at carbon atoms thereof by two R₆O—CO or R₆O—CO—C₁₋₄-alkyl groups,
(l) a pyrrolidinyl, piperidinyl, or hexahydroazepinyl group substituted at the 1 position by an R₆O—CO—C₁₋₄-alkyl, bis-(R₆O—CO)—C₁₋₄-alkyl, (R₇O—PO—OR₈)—C₁₋₄-alkyl, or (R₇O—PO—R₉)—C₁₋₄-alkyl group,
(m) a pyrrolidinyl, piperidinyl, or hexahydroazepinyl group substituted at the 1 position by an R₆O—CO—C₁₋₄-alkyl or bis-(R₆O—CO)—C₁₋₄-alkyl group, wherein the 5- to 7-membered rings thereof in each case are additionally substituted at carbon atoms thereof by one or two R₆O—CO or R₆O—CO—C₁₋₄-alkyl groups,
(n) a 2-oxomorpholino group optionally substituted by 1 to 4 C₁₋₂-alkyl groups,
(o) a 2-oxomorpholinyl group substituted at the 4 position by a hydrogen atom, or by a C₁₋₄-alkyl or R₆O—CO—C₁₋₄-alkyl group, wherein the 2-oxomorpholinyl group is linked to a carbon atom of the group C, and
(p) an R₁₁NR₅ group, or
C together with D are a group consisting of:
(a) a hydrogen atom,
(b) a C₁₋₄-alkoxy group,
(c) a C₂₋₄-alkoxy group substituted at a position other than position 1 by a hydroxy, C₁₋₄-alkoxy, amino, C₁₋₄-alkylamino, di-(C₁₋₄-alkyl)-amino, pyrrolidino, piperidino, morpholino, piperazino, or 4-(C₁₋₄-alkyl)-piperazino group,
(d) a C₁₋₄-alkoxy group substituted by a pyrrolidinyl or piperidinyl group substituted at the 1 position by R₁₀,
(e) a C₁₋₄-alkoxy group substituted by an R₆O—CO group, and
(f) a C₄₋₇-cycloalkoxy or C₃₋₇-cycloalkyl-C₁₋₄-alkoxy group;
R_{c} and R_{d} in each case are a hydrogen atom;
Rₑ and R_{f}, which are identical or different, in each case are a hydrogen atom or a C₁₋₄-alkyl group;
R_{g} is a C₁₋₄-alkyl, C₃₋₆-cycloalkyl, C₁₋₄-alkoxy, or C₅₋₆-cycloalkoxy group;
R₅ is a hydrogen atom,
a C₁₋₄-alkyl group optionally substituted by an R₆O—CO group,
a C₂₋₄-alkyl group substituted at a position other than position 1 by a hydroxy or C₁₋₄-alkoxy group, or
a C₃₋₆-cycloalkyl or C₃₋₆-cycloalkyl-C₁₋₃-alkyl group;
R₆, R₇, and R₈, which are identical or different, in each case are a hydrogen atom,
a C₁₋₈-alkyl group optionally substituted at a position other than position 1 by a hydroxy, C₁₋₄-alkoxy, or di-(C₁₋₄-alkyl)-amino group or by a 4- to 7-membered alkyleneimino group, wherein a methylene group at the 4 position of the 6- to 7-membered alkyleneimino group is optionally replaced by an oxygen atom or by an N—(C₁₋₂-alkyl)-imino group,
a C₄₋₆-cycloalkyl group,
a C₃₋₅-alkenyl or C₃₋₅-alkynyl group, wherein the unsaturated moiety is not linked to the oxygen atom,
a C₃₋₆-cycloalkyl-C₁₋₄-alkyl, aryl, aryl-C₁₋₄-alkyl, or R_{g}CO—O—(RₑCR_{f}) group, wherein
R₉ is a C₁₋₄-alkyl group;
R₁₀ is a hydrogen atom, or a methyl or ethyl group;
R₁₁ is a 2-oxotetrahydrofuran-3-yl, 2-oxotetrahydrofuran-4-yl, 2-oxotetrahydropyran-3-yl, 2-oxotetrahydropyran-4-yl, or 2-oxotetrahydropyran-5-yl group optionally substituted by one or two methyl groups;
R₁₂ is a cyano, C₁₋₂-alkoxycarbonyl, aminocarbonyl, C₁₋₂-alkylaminocarbonyl, di-(C₁₋₂-alkyl)-aminocarbonyl, C₁₋₂-alkylsulfenyl, C₁₋₂-alkylsulfinyl, C₁₋₂-alkylsulfonyl, hydroxy, nitro, amino, C₁₋₄-alkylamino, or di-(C₁₋₄-alkyl)-amino group; and
R₁₃ is a fluorine, chlorine, bromine, or iodine atom, or a C₁₋₂-alkyl, trifluoromethyl, or C₁₋₂-alkoxy group, or two groups R₁₃, if they are bound to adjacent carbon atoms, together are a C₃₋₅-alkylene, methylenedioxy, or 1,3-butadien-1,4-ylene group,
or a tautomer, stereoisomer, or salt thereof.

4. The compound of formula (I) according to claim 1, wherein:
Rₐ is a hydrogen atom,
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, or bromine atom, or
(ii) a methyl, trifluoromethyl, methoxy, ethynyl, or cyano group, and
R₃ is a hydrogen atom;
X is a nitrogen atom;
A is an —O—C₁₋₄-alkylene or —O—CH₂—CH(OH)—CH₂ group, wherein the oxygen atom thereof in each case is linked to the bicyclic heteroaromatic moiety of formula (I);
B is a group consisting of:
(a) an R₆O—CO-alkylene-NR₅ group wherein the alkylene moiety, which is straight-chained and contains 1 or 2 carbon atoms, is additionally optionally substituted by an R₆O—CO or R₆O—CO-methyl group;
(b) a pyrrolidino or piperidino group substituted by an R₆O—CO or R₆O—CO—C₁₋₂-alkyl group,
(c) a pyrrolidino or piperidino group substituted by two R₆O—CO or R₆O—CO—C₁₋₂-alkyl groups,
(d) a piperazino group substituted at the 4 position by R₁₀ and additionally at a cyclic carbon atom thereof by an R₆O—CO or R₆O—CO—C₁₋₂-alkyl group,
(e) a piperazino group substituted at the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(f) a piperazino group substituted at the 4 position by an R₆O—CO—C₁₋₂-alkyl group and additionally at a cyclic carbon atom thereof by an R₆O—CO or R₆O—CO—C₁₋₂-alkyl group,
(g) a morpholino group substituted by an R₆O—CO or R₆O—CO—C₁₋₂-alkyl group,
(h) a pyrrolidinyl or piperidinyl group substituted at the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(i) a 2-oxomorpholino group optionally substituted by 1 or 2 methyl groups,
(j) a 2-oxomorpholinyl group substituted at the 4 position by a methyl, ethyl, or R₆O—CO—C₁₋₂-alkyl group, wherein the 2-oxomorpholinyl group is linked to a carbon atom of the group A, and
(k) a R₁₁N(C₁₋₂-alkyl) group, or
A and together with B are a group consisting of:
(a) a hydrogen atom, or a methoxy, ethoxy, or 2-methoxyethoxy group,
(b) a C₁₋₂-alkoxy group substituted by an R₆O—CO group, and
(c) a C₄₋₆-cycloalkoxy or C₃₋₆-cycloalkyl-C₁₋₃-alkoxy group;
C is an —O—C₁₋₄-alkylene or —O—CH₂—CH(OH)—CH₂ group, wherein the oxygen atom thereof in each case is linked to the bicyclic heteroaromatic moiety of formula (I),
D is a group consisting of:
(a) an R₆O—CO-alkylene-NR₅ group wherein the alkylene moiety, which is straight-chained and contains 1 or 2 carbon atoms, is additionally optionally substituted by an R₆O—CO or R₆O—CO-methyl group,
(b) a pyrrolidino or piperidino group substituted by an R₆O—CO or R₆O—CO—C₁₋₂-alkyl group,
(c) a pyrrolidino or piperidino group substituted by two R₆O—CO or R₆O—CO—C₁₋₂-alkyl groups,
(d) a piperazino group substituted at the 4 position by R₁₀ and additionally substituted at a cyclic carbon atom thereof by an R₆O—CO or R₆O—CO—C₁₋₂-alkyl group,
(e) a piperazino group substituted at the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(f) a piperazino group substituted at the 4 position by an R₆O—CO—C₁₋₂-alkyl group and additionally substituted at a cyclic carbon atom thereof by an R₆O—CO or R₆O—CO—C₁₋₂-alkyl group,
(g) a morpholino group substituted by an R₆O—CO or R₆O—CO—C₁₋₂-alkyl group,
(h) a pyrrolidinyl or piperidinyl group substituted at the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(i) a 2-oxomorpholino group optionally substituted by 1 or 2 methyl groups,
a 2-oxomorpholinyl group substituted at the 4 position by a methyl, ethyl, or R₆O—CO—C₁₋₂-alkyl group, wherein the 2-oxomorpholinyl group is linked to a carbon atom of the group C, and
(k) a R₁₁N(C₁₋₂-alkyl) group, or
C together with D are a group consisting of:
(a) a hydrogen atom, or a methoxy, ethoxy, or 2-methoxyethoxy group,
(b) a C₁₋₂-alkoxy group substituted by an R₆O—CO group, and
(c) a C₄₋₆-cycloalkoxy or C₃₋₆-cycloalkyl-C₁₋₃-alkoxy group;
R_{c} and R_{d} in each case are a hydrogen atom;
Rₑ is a hydrogen atom or a C₁₋₄-alkyl group;
R_{f} is a hydrogen atom;
R_{g} is a C₁₋₄-alkyl, cyclopentyl, cyclohexyl, C₁₋₄-alkoxy, cyclopentyloxy, or cyclohexyloxy group;
R₅ is a hydrogen atom,
a C₁₋₂-alkyl group optionally substituted by an R₆O—CO group,
a C₂₋₄-alkyl group substituted at a position other than position 1 by a hydroxy group, or
a C₃₋₆-cycloalkyl or C₃₋₆-cycloalkylmethyl group;
R₆ is a hydrogen atom, or a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group;
R₇ and R₈, which are identical or different, in each case are a hydrogen atom, or a methyl, ethyl, phenyl, benzyl, 5-indanyl, or R_{g}CO—O—(RₑCR_{f}) group;
R₉ is a methyl or ethyl group;
R₁₀ is a hydrogen atom, or a methyl or ethyl group; and
R₁₁ is a 2-oxotetrahydrofuran-3-yl or 2-oxotetrahydrofuran-4-yl group,
or a tautomer, stereoisomer, or salt thereof.

5. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are a hydrogen, fluorine, chlorine, or bromine atom, or a methyl, trifluoromethyl, methoxy, ethynyl, or cyano group, and R₃ is a hydrogen atom;
X is a nitrogen atom;
A is an —O—C₁₋₄-alkylene or —O—CH₂—CH(OH)—CH₂ group, wherein the oxygen atom thereof in each case is linked to the bicyclic heteroaromatic moiety of formula (I);
B is a group consisting of:
(a) a R₆O—CO-alkylene-NR₅ group wherein the alkylene moiety, which is straight-chained and contains 1 or 2 carbon atoms, is additionally optionally substituted by an R₆O—CO or R₆O—CO-methyl group,
(b) a pyrrolidino or piperidino group substituted by an R₆O—CO or R₆O—CO—C₁₋₂-alkyl group,
(c) a pyrrolidino or piperidino group substituted by two R₆O—CO or R₆O—CO—C₁₋₂-alkyl groups,
(d) a piperazino group substituted at the 4 position by R₁₀ and additionally substituted at a cyclic carbon atom thereof by an R₆O—CO or R₆O—CO—C₁₋₂-alkyl group,
(e) a piperazino group substituted at the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(f) a piperazino group substituted at the 4 position by an R₆O—CO—C₁₋₂-alkyl group and additionally substituted at a cyclic carbon atom thereof by an R₆O—CO or R₆O—CO—C₁₋₂-alkyl group,
(g) a morpholino group substituted by an R₆O—CO or R₆O—CO—C₁₋₂-alkyl group,
(h) a pyrrolidinyl or piperidinyl group substituted at the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(i) a 2-oxomorpholino group optionally substituted by 1 or 2 methyl groups,
a 2-oxomorpholinyl group substituted at the 4 position by a methyl, ethyl, or R₆O—CO—C₁₋₂-alkyl group, wherein the 2-oxomorpholinyl group is linked to a carbon atom of the group A, and
(k) a R₁₁N(C₁₋₂-alkyl) group, or
C together with D are a group consisting of a hydrogen atom, and a methoxy, ethoxy, 2-methoxyethoxy, C₄₋₆-cycloalkoxy, and C₃₋₆-cycloalkyl-C₁₋₃-alkoxy group;
R_{c} and R_{d} in each case are a hydrogen atom;
Rₑ is a hydrogen atom or a C₁₋₄-alkyl group;
R_{f} is a hydrogen atom;
R_{g} is a C₁₋₄-alkyl, cyclopentyl, cyclohexyl, C₁₋₄-alkoxy, cyclopentyloxy, or cyclohexyloxy group;
R₅ is a hydrogen atom,
a C₁₋₂-alkyl group optionally substituted by an R₆O—CO group,
a C₂₋₄-alkyl group substituted at a position other than position 1 by a hydroxy group, or
a C₃₋₆-cycloalkyl or C₃₋₆-cycloalkylmethyl group;
R₆ is a hydrogen atom, or a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group;
R₇ and R₈, which are identical or different, in each case are a hydrogen atom, or a methyl, ethyl, phenyl, benzyl, 5-indanyl, or R_{g}CO—O—(RₑCR_{f}) group;
R₉ is a methyl or ethyl group;
R₁₀ is a hydrogen atom, or a methyl or ethyl group; and
R₁₁ is a 2-oxotetrahydrofuran-3-yl or 2-oxotetrahydrofuran-4-yl group,
or a tautomer, stereoisomer, or salt thereof.

6. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, or bromine atom, or
(ii) a methyl, trifluoromethyl, methoxy, ethynyl, or cyano group, and
R₃ is a hydrogen atom;
X is a nitrogen atom;
A together with B are a group consisting of: a hydrogen atom, or a methoxy, ethoxy, 2-methoxyethoxy, C₄₋₆-cycloalkoxy, or C₃₋₆-cycloalkyl-C₁₋₃-alkoxy group;
C is an —O—C₁₋₄-alkylene or —O—CH₂—CH(OH)—CH₂ group, wherein the oxygen atom thereof in each case is linked to the bicyclic heteroaromatic moiety of formula (I);
D is a group consisting of:
(a) R₆O—CO-alkylene-NR₅ group wherein the alkylene moiety, which is straight-chained and contains 1 or 2 carbon atoms, is additionally optionally substituted by an R₆O—CO or R₆O—CO-methyl group,
(b) a pyrrolidino or piperidino group substituted by an R₆O—CO or R₆O—CO—C₁₋₂-alkyl group,
(c) a pyrrolidino or piperidino group substituted by two R₆O—CO or R₆O—CO—C₁₋₂-alkyl groups,
(d) a piperazino group substituted at the 4 position by R₁₀ and additionally at a cyclic carbon atom thereof by an R₆O—CO or R₆O—CO—C₁₋₂-alkyl group,
(e) a piperazino group substituted at the 4 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(f) a piperazino group substituted at the 4 position by an R₆O—CO—C₁₋₂-alkyl group and additionally substituted at a cyclic carbon atom thereof by an R₆O—CO or R₆O—CO—C₁₋₂-alkyl group,
(g) a morpholino group substituted by an R₆O—CO or R₆O—CO—C₁₋₂-alkyl group,
(h) a pyrrolidinyl or piperidinyl group substituted at the 1 position by an R₆O—CO—C₁₋₄-alkyl, bis-(R₆O—CO)—C₁₋₄-alkyl, (R₇O—PO—OR₈)-methyl, or (R₇O—PO—R₉)-methyl group,
(i) a 2-oxomorpholino group optionally substituted by 1 or 2 methyl groups,
(j) a 2-oxomorpholinyl group substituted at the 4 position by a methyl, ethyl, or R₆O—CO—C₁₋₂-alkyl group, wherein the 2-oxomorpholinyl group is linked to a carbon atom of the group C, and
(k) a R₁₁N(C₁₋₂-alkyl) group;
R_{c} and R_{d} in each case are a hydrogen atom;
Rₑ is a hydrogen atom or a C₁₋₄-alkyl group;
R_{f} is a hydrogen atom;
R_{g} is a C₁₋₄-alkyl, cyclopentyl, cyclohexyl, C₁₋₄-alkoxy, cyclopentyloxy, or cyclohexyloxy group;
R₅ is a hydrogen atom,
a C₁₋₂-alkyl group optionally substituted by an R₆O—CO group,
a C₂₋₄-alkyl group substituted at position 2 by a hydroxy group, or
a C₃₋₆-cycloalkyl or C₃₋₆-cycloalkylmethyl group;
R₆ is a hydrogen atom, or
a C₁₋₆-alkyl, cyclopentyl, cyclopentylmethyl, cyclohexyl, cyclohexylmethyl, phenyl, benzyl, 5-indanyl, or R_{g}CO—O—(RₑCR_{f}) group;
R₇ and R₈, which are identical or different, in each case are a hydrogen atom, or a methyl, ethyl, phenyl, benzyl, 5-indanyl, or R_{g}CO—O—(RₑCR_{f}) group;
R₉ is a methyl or ethyl group;
R₁₀ is a hydrogen atom, or a methyl or ethyl group;
R₁₁ is a 2-oxotetrahydrofuran-3-yl or 2-oxotetrahydrofuran-4-yl group,
or a tautomer, stereoisomer, or salt thereof.

7. The compound of formula (I) according to claim 1, wherein:
Rₐ is a hydrogen atom;
R_{b} is a phenyl group wherein the phenyl nucleus is substituted in each case by R₁, R₂, and R₃, wherein:
R₁ and R₂, which are identical or different, each are a hydrogen, fluorine, chlorine, or bromine atom, and
R₃ is a hydrogen atom;
X is a nitrogen atom;
A is an —O—C₁₋₄-alkylene or —O—CH₂—CH(OH)—CH₂ group, wherein the oxygen atom thereof in each case is linked to the bicyclic heteroaromatic moiety of formula (I);
B is a group consisting of:
(a) an R₆—CO—CH₂—NR₅ group;
(b) a pyrrolidino or piperidino group substituted by an R₆O—CO group,
(c) a piperazino group substituted at the 4 position by an R₆O—CO—CH₂ or bis-(R₆O—CO)—C₁₋₃-alkyl group,
(d) a pyrrolidinyl or piperidinyl group substituted at the 1 position by an R₆O—CO—CH₂ group,
(e) a 2-oxomorpholino group optionally substituted by one or two methyl groups, or
(f) a R₁₁N(C₁₋₂-alkyl) group, or
C together with D is a group consisting of a methoxy, C₄₋₆-cycloalkoxy, or C₃₋₆-cycloalkylmethoxy group;
R_{c} and R_{d} in each case are a hydrogen atom;
R₅ is a hydrogen atom or a methyl group optionally substituted by an R₆O—CO group, or
a C₂₋₄-alkyl group substituted at a position other than position 1 by a hydroxy group;
R₆ is a hydrogen atom, or a methyl or ethyl group;
R₁₁ is a 2-oxotetrahydrofuran-3-yl or 2-oxotetrahydrofuran-4-yl group, and
or a tautomer, stereoisomer, or salt thereof.

8. A compound selected from the group consisting of:
(1) 4-(3-chloro-4-fluorophenylamino)-6-{3-[4-(methoxycarbonylmethyl)-1-piperazinyl]propyloxy}-7-methoxyquinazoline;
(2) 4-[(3-bromophenyl)amino]-6-(2-{4-[(ethoxycarbonyl)methyl]piperazin-1-yl}ethoxy)-7-methoxyquinazoline;
(3) (S)-4-[(3-bromophenyl)amino]-6-[3-(2-methoxycarbonylpyrrolidin-1-yl)propyloxy]-7-methoxyquinazoline;
(4) 4-[(3-bromophenyl)amino]-6-(3-{4-[(ethoxycarbonyl)methyl]piperazin-1-yl}-2-hydroxypropyloxy)-7-methoxyquinazoline;
(5) (S)-4-[(3-bromophenyl)amino]-6-({1-[(ethoxycarbonyl)methyl]pyrrolidine-2-yl}methoxy)-7-methoxyquinazoline; and
(6) 4-[(3-bromophenyl)amino]-6-(2-{4-[1,2-bis(methoxycarbonyl)ethyl]piperazin-1-yl}ethoxy)-7-methoxyquinazoline,
and the salts thereof.

9. The compound according to one of claims 1 to 8, wherein the compound is a physiologically acceptable salt.

10. A pharmaceutical composition comprising an effective amount of a compound of formula (I) according to one of claims 1 to 8 and an inert carrier or diluent.

11. A pharmaceutical composition comprising an effective amount of a compound of formula (I) according to claim 9 and an inert carrier or diluent.

12. A method for treatment or prophylaxis of benign or malignant tumors, diseases of the airways and lungs, polyps, diseases of the gastrointestinal tract, bile duct, gall bladder, kidneys, and skin, in a host in need of such treatment or prophylaxis, which method comprises administering the host an effective amount of a compound according to one of claims 1 to 8.

13. A method for treatment or prophylaxis of benign or malignant tumors, diseases of the airways and lungs, polyps, diseases of the gastrointestinal tract, bile duct, gall bladder, kidneys, and skin, in a host in need of such treatment or prophylaxis, which method comprises administering the host an effective amount of a compound according to claim 9.
